(19) United States
(12) Patent Application Publication
(10) Pub. No.: US 2014/0018732 A1
(43) Pub. Date: Jan. 16, 2014

(54) Apparatus and Methods for Accessing and Treating a Body Cavity, Lumen, or Ostium

(71) Applicants: Celso J. Bagaoisan, Union City, CA (US); Glen Gong, San Carlos, CA (US); Suresh Pai, Mountain View, CA (US); Scott Robert Sershen, Redwood City, CA (US)

(72) Inventors: Celso J. Bagaoisan, Union City, CA (US); Glen Gong, San Carlos, CA (US); Suresh Pai, Mountain View, CA (US); Scott Robert Sershen, Redwood City, CA (US)

(73) Assignee: SPOTLIGHT TECHNOLOGY PARTNERS LLC, Mountain View, CA (US)

(21) Appl. No.: 13/978,229

(22) PCT Filed: Jan. 4, 2012
(86) PCT No.: PCT/US2012/020203

§ 371 Date: Sep. 25, 2013

Related U.S. Application Data
(60) Provisional application No. 61/431,331, filed on Jan. 10, 2011.

(51) Int. Cl. A61M 25/01

(52) U.S. Cl. CPC A61M 25/0147
USPC 604/95.04

(57) ABSTRACT

Among the various embodiments, objects and features of the present invention may generally be noted catheter systems which simplify and ease access to one or more target anatomies in various medical procedures thereby reducing procedure time and associated costs.

### CROSS-REFERENCE TO RELATED APPLICATIONS

Pursuant to 35 U.S.C. §119(e), this application claims priority to U.S. Provisional Application No. 61/431,331 filed on Jan. 10, 2011, the disclosures of which is herein incorporated by reference in its entirety.

### FIELD OF THE INVENTION

The inventions of this specification relate generally to medical devices or more specifically to steerable elongate guide and catheter systems with a tip indication mechanism that can be transformed inside a human or animal body into various geometric shapes without the aid of visualization of the transformed segment to treat or aid in the treatment of a body cavity, lumen, ostium. The invention also includes embodiments that describe the methods of use for said systems.

### BACKGROUND

Angioplasty and stenting are commonly used for the treatment of stenosed renal arteries. In patients suffering from stenotic lesions in these arteries, the take-off angles of the renal arteries relative to the aorta can vary significantly from patient to patient. Frequently the disease can also occur bilaterally (i.e. in both the left and right renal arteries) and physicians are inclined to treat both arteries in the same procedural setting. Selective angiography and subsequent cannulization of the renal arteries is accomplished using commonly available pre-shaped guide catheters and guide sheaths that are typically advanced to the target renal artery from a vascular access point in the patient's femoral artery located near the groin. Femoral artery access is obtained using the Seldinger technique after which an intravascular sheath is placed into the artery lumen allowing for passage of instrumentation. Typically in these procedures, a guide catheter with a pre-set distal shape is advanced to the vicinity of the diseased renal artery. It is not uncommon to find that the vessels that must be traversed by the guide catheter in order to reach the target renal artery are highly tortuous and ectatic in nature. Physician preference and experience combined with available diagnostic imaging data typically dictates the pre-set shape that will be chosen by the physician in the procedure. Due to the flow dynamics of the parent artery (i.e. the aorta), it is important in these procedures that the guide catheter tip or distal segment generally seats closely to the ostium of the artery so as to limit wash out or loss of injected contrast agent through the lumen of the aorta precluding the ability to obtain clear visualization of the renal artery using traditional angiographic methods and equipment. In addition, it is important to avoid excessive manipulation inside the aorta since patients with peripheral artery disease often have plaques on the aortic wall and dragging a guide catheter across these plaques could create or exacerbate the risk of embolization. Thus, it would desirable to provide a means to access the target renal artery or arteries with a catheter system that maintains a generally straight configuration during insertion and positioning in the body thereby mitigating the possibility of dragging the guide catheter tip along the wall of the aorta. Upon locating the guide tip or distal segment near the vicinity of the of the target artery it would then be advantageous to have a means to steer or aim a catheter tip in the trajectory of the vessel take-off to enable placement of a guidewire and subsequent instrumentation of the vessel ostium or lumen and remove the limitations imposed by a pre-set guide shape. Ideally, the shape transformation of the distal tip or segment of the guide could be accomplished via a simple, easy-to-use indicator at the proximal end of the guide system eliminating the need for visual confirmation of the transformation at the distal end of the system. Additionally, it would also be desirable to change or steer the same guide into the lumen of the contralateral vessel (i.e. an alternate anatomical position or target) during the same medical procedure. The take-off angle of the contralateral vessel relative to the aorta and the requirements to access this vessel could differ significantly from those used to access the ipsilateral artery. Thus, a means to easily customize access to the artery in situ would be highly desirable to reduce procedure time and limit procedural steps and cumbersome maneuvers such as over the wire guide catheter exchanges. It would also be desirable and provide great utility to have a means to access the target artery with more ease via a system that could steer and more easily navigate through significant vessel tortuosity encountered while accessing the target artery or arteries in these types of procedures. The ability to make changes to or to transform the catheter shape in the same procedural setting without the need for visual confirmation of the shape change would also have great utility reducing procedure times and minimizing exposure to radiation in these procedures.

The coronary arteries of the heart are accessed by cardiologists using similar equipment and methods as those described for the renal arteries. The coronary arteries (i.e. left main, right coronary artery and the circumflex artery) can be isolated or sometimes occurs in multiple vessels simultaneously. Patients with multi-vessel disease often require diagnostic and interventional treatment procedures of the various lesions in the same setting. The ostia of the coronary arteries emanate from the aortic sinus at unique positions in the aortic sinus bulb. These positions can be very difficult for the cardiologist to navigate using current tools available in the cardiology device arsenal. The take-off angles or complex patient anatomies of the various vessels can also vary widely further adding further challenge to the placement of the guidewires and guide catheters typically used in these procedures. Over the past two decades, pre-set guide catheter shapes or geometries have become available that improve the capability of the physician to access and cannulate the coronary arteries. However, this access can still be cumbersome and time consuming as the physician is forced to render the pre-set shape workable for the procedure since the shape cannot be modified pre-procedurally or peri-procedurally. Like renal artery angioplasty and stenting procedures, the choice of which pre-set shape to use is based on the physician's preference, experience combined with previously taken diagnostic images that may be available. As before, in cases where the chosen pre-set shape fails to meet the physician's expectations, it would be highly desirable to have the capability to redirect or steer or aim the tip of the guide toward the target body cavity, lumen, artery, or artery ostium. It would also be valuable and desirable to be able to utilize the same catheter to access the other coronary arteries and/or ostia in the same procedural setting obviating the need for cumbersome over the wire guide catheter exchanges. Finally, it would be desirable if all of the customization and steering steps (i.e. shape change of the catheter's distal segment or tip) could be accomplished without the need for visual confirmation reducing procedural times and exposure to radiation.

Many peripheral stenting, angioplasty and other interventional procedures will often employ a technique where the guide catheter is placed from an access point in the femoral artery of the opposite leg. This type of access provides facilitates better pushability and allows the physician to better manipulate the devices and instruments typically used in procedures to be completed on the opposite side. Access from the opposite leg is also common when the diseased blood vessel targeted for treatment is too near where the intravascular sheath would need to be placed not allowing enough room to place the tools and effectively use the devices & instruments from the same side. As mentioned previously, vessel tortuosity in the vicinity of the femoral artery access and to the target artery can be significant and placement of the guide can present time-consuming challenges and safety risks (such as vessel perforation or trauma) to a procedure. Anatomically, the terminal aorta bifurcates into the origin of the two common iliac arteries. It is not uncommon for the take-off angles of the iliac arteries from the terminal aorta to be very steep assuming almost an upside down “V” shape with a very acute inside angle. As with the other previously mentioned procedures, access up and over the aortic arch typically involves placement of a guidewire over the arch and down the femoral artery segment of the opposite leg over which a guide catheter or sheath is advanced coaxially (over the wire) to the treatment target. The challenge with crossing the aortic arch is that the guide catheter will often prefer to advance up the aorta instead over the wire into the opposite iliac and the guidewire is often displaced in this maneuver. The undesirable displacement of the pre-positioned guidewire forces the physician to back up the guide catheter and attempt to recannulate the guidewire back to the target artery adding procedure time and undesirable tedium. Thus it would be highly desirable to be able to provide access up and over the aortic arch by steering or aiming the tip of the guide catheter into a trajectory that helps aim the access and/or treatment catheter body towards the origin of the contralateral common iliac artery. Once the tip of the catheter enters the common iliac origin of the contralateral artery, a more effective push force on the system over the guidewire would be enabled. As with the other vascular procedures described in this specification, the ability to customize the geometry of the catheter to enable access is highly desirable and removes the limits imposed by a pre-set shaped guide catheter or guide sheath. Furthermore, if this customization could be completed without the need for visual confirmation of the shape change or transformation, procedure times could be significantly reduced. Also, like the other examples mentioned in this specification, exposure to radiation could be reduced since the need to use fluoroscopic imaging for confirmation could be eliminated. In general, patients suffering from peripheral artery disease in the legs would benefit from the invention. The physician would be provided with means to customize the device tip as required to select target arteries (e.g. the origin of the internal iliac artery from within the common iliac artery). The desirable properties of a steerable catheter that could be modified without visual confirmation would find great utility in these procedures.

Access to the neuro-vasculature or vessels that feed the brain can be difficult using the currently available devices and systems. These vessels include the brachiocephalic or innominate artery, left common carotid artery and the left subclavian artery that emanate off of the aortic arch. In one variation, called the bovine arch, the left common carotid artery originates off the innominate artery instead of the aorta. The successful cannulization of these arteries depends upon initial access with guidewires and then careful placement of guide catheters or guide sheaths coaxially over those guidewires. Stenting of the carotid arteries has become more prevalent over the last decade so the demand for simpler, easier access to the internal carotid arteries has increased. The typical challenge with pushing a guide catheter into the innominate artery is related to the origination of the innominate artery from the ascending aorta. As the guide catheter is pushed or advanced, the force vector on the guide catheter is such that the preferred path of least resistance is to advance the guide catheter towards the heart (i.e. away from the target artery). Thus it is clear that it would be highly desirable to have means to direct or steer or aim the access and/or treatment catheter tip towards the origin of the innominate artery. Once the tip cannulates the origin the next steps to push and advance the system over the guidewire would be greatly eased. The same would hold true for placement of devices into other targets in the neuro-vasculature. In the case of the bovine arch, it would be preferable to have the capability to customize or redirect the tip of the catheter towards the right common carotid take-off or origin after successful access or cannulization of the innominate artery. Similar to the issues mentioned for innominate artery access, the typical guide catheter will have a preference or tendency to be to pushed or advanced forward towards the subclavian artery likely displacing the guidewire. Thus, again it is clear that there would be great utility to have the capability to variably modify the geometry of the access and/or treatment catheter tip multiple times and at the discretion of the physician during the same procedure. The ability to make these shape changes to the distal segment or tip reliably without the need for visualization would as mentioned previously for the other applications be valuable.

It is clear that all of the previously mentioned examples where the current invention provides value can be used not only for selective catheterization procedures to produce diagnostic images, but also for interventional procedures such as stenting, atherectomy, other vascular interventional procedures and the like.

Catheter based procedures that map and when desired ablate the electrical signaling pathways inside the heart also could benefit from a system that provides improved steering or directionality. Electrophysiologists identify precise segments of tissue for example in the left atrium where a device needs to be positioned and as such a catheter system that enable access to and direction of instruments towards these segments would be highly desirable. These procedures often employ guide catheters that are placed in the venous system and access the left atrium through a trans-septal puncture from the right atrium. As such, the guide catheter must traverse significant tortuosity to ultimately gain successful entry into the left atrium. As before, having the ability to peri-procedurally customize the shape of the access and/or treatment catheter's distal segment or tip could help a physician navigate vessel tortuosity while the eliminating the need for confirmation visually of the tip shape change would also be desirable to ease access, reduce procedure time and minimize radiation exposure.

Most vascular diagnostic and interventional catheter based procedures start with retrograde punctures made in the femoral or radial artery using the well known Seldinger technique after which a standard intravascular sheath is coaxially threaded into the vessel lumen over a guidewire. These sheaths are made of single lumen tubes connected to a hub housing a valve through which maintain a fluid tight seal prevent leakage of blood while simultaneously permitting the passage of instruments. During insertion of the sheath, another component called a dilator is inserted coaxially within the lumen of the sheath to give the sheath the required rigidity and to allow it to be pushed over the wire into the vessel lumen. The dilator design provides a gentler, more tapered, less traumatic leading edge for the sheath to traverse through the soft tissue bed as it is advanced towards the femoral (or radial) artery and through puncture into the vessel lumen. The angle of entry varies depending on the patient's anatomy and the physician will often have to vary this angle to blindly locate the anterior portion of the artery with the needle. Steep entry angles often create challenges for placement of intravascular sheaths due to the inherent stiffness of the sheath and dilator combination. The stiffness combined with the steep entry angle can force the sheath to not track effectively down the wire and into the vessel and instead force the dilator tip towards into the opposite artery wall leading to a potential for trauma and/or damage to the sheath lumen or body. Thus it may be preferable to have means to steer or direct the sheath into the preferred trajectory. The same applies in the case of antegrade vessel punctures. The sheath can have such a steep entry angle that the physician has difficulty effectively and safely placing the sheath into the target artery.

Minimally invasive surgical procedures are desirable because such procedures can reduce pain and provide relatively quick recovery times as compared with conventional open medical procedures. Many minimally invasive procedures are performed through one or more ports commonly known as trocars. A laparoscope, which may or may not include a camera, may be used through one of these ports for visualization of the anatomy and surgical instruments may be used simultaneously through other ports. Such devices and procedures permit a physician to position, manipulate, and view anatomy, surgical instruments and accessories inside the patient through a small access opening in the patient's body. Some examples of surgical procedures performed using these minimally invasive techniques include biliary stenting, gastric bypass, fundoplicaiton, lap band surgery, GERD interventions, tissue and tumor resection.

Still less invasive procedures include those that are performed through insertion of an endoscope through a natural body orifice to a treatment region. Examples of these approaches include colonoscopy, hysteroscopy, cystoscopy, and esophagogastroduodenoscopy. Many of these procedures employ the use of a flexible endoscope during the procedure. Flexible endoscopes often have a flexible, steerable articulating section near the distal end that can be controlled by the user by utilizing controls at the proximal end. Treatment or diagnosis may be completed intralumenally, such as polypectomy or gastroscopy.

Some flexible endoscopes are relatively small range from 1 mm to 3 mm in diameter, and may have no internal working channel. Other flexible endoscopes, including gastroscopes and colonoscopes, have integral working channels having a diameter of about 2.0 to 3.5 mm for the purpose of introducing and removing medical devices and other accessory devices to perform diagnosis or therapy within the patient. As a result, the accessory devices used by a physician can be limited in size by the diameter of the accessory channel of the scope used. Additionally, the physician may be limited to a single accessory device when using the standard endoscope having one working channel.

Over the years, a variety sheaths accommodating endoscopes have been developed. Some sheath arrangements are substantially steerable by means of control knobs supported on a housing assembly. Regardless of the type of surgery involved and the method in which the endoscope is inserted into the body, the surgeons and surgical specialists performing such procedures have generally developed skill sets and approaches that rely on anatomical alignment for both visualization and tissue manipulation purposes. However, due to various limitations of those prior sheath arrangements, the surgeon may often times be forced to view the surgical site in such a way that is unnatural and thereby difficult to follow and translate directional movement within the operating theater to corresponding directional movement at the surgical site. Moreover, such prior devices are not particularly well-equipped to accommodate and manipulate multiple surgical instruments and tools within the surgical site without having to actually move and reorient the endoscope.

Consequently a significant need exists for an alternative to conventional sheaths for use with endoscopes and other surgical tools and instruments that can be advantageously manipulated and oriented and which can accommodate a variety of different tools and instruments and facilitate movement and reorientation of such tools and instruments without having to reorient or move the outer sheath.

Endoscopy is expanding its role from diagnostics and simple therapeutics to advanced surgical techniques applicable to disease of the gastrointestinal tract and peritoneal structures. Natural orifice transluminal endoscopic surgery (NOTES) is an emerging alternative to conventional abdominal surgery that combines laparoscopic and endoscopic techniques in order to access the peritoneal cavity by means of mouth, anus, the umbilicus, or possibly vagina thereby avoiding external incisions and their related complications. Various procedures are possible using NOTES, such as cholecystectomy, appendectomy, full-thickness stomach resection, splenectomy, gastrointestinal (GI) anastomoses, and peritoneoscopy.

The advantages of NOTES over conventional surgery and or laparoscopy include the elimination of complications including pain, hernias and external wound infections caused by surgical incisions. The NOTES also offers the benefit of reducing the amount of trauma to the surrounding tissue, which may shorten a hospital stay. Though NOTES may help minimize the complications associated with traditional surgical techniques it is a challenges to perform surgical procedures through small natural orifices without instruments specifically developed for the procedures. The endoscopes used in the NOTES must have adequate resolution, channel size, and the ability to lock into position inside the peritoneum, as the instruments must have the same or better capabilities of standard laparoscopic instruments. Furthermore, the need for tissue triangulation has to be accomplished from a single instrument and so devices with multiple heads have to be developed.

Pulmonologists use bronchoscopes to inspect the interior surfaces of the lungs and trachea to perform a variety of diagnostic and surgical procedures. Devices, such as biopsy forceps, brushes, needles, catheters, stents, coils, one way valves, steam, energy, glues/sealants, can be passed through the length of the bronchoscope via the working channel into a patient's lungs to obtain tissue samples. For example, a biopsy needle may be inserted into a patient's lung via the working channel of a flexible bronchoscope. Once the needle is in place at the distal end of the bronchoscope, the pulmonologist can use the needle to biopsy a lymph node in the mediastinal space adjacent the bronchus in which the bronchoscope is placed. There is a growing need for larger sized and multiple working channels to perform more advanced interventional pulmonary procedures such as minimally invasive lung volume reduction surgery were one way valves, lung coil devices, and sealants are deployed to help reduction the volume of lung thereby restoring diaphragm function.

Endourology and laparoscopy treats a wide variety of urologic issues involving the adrenal gland, kidney, ureter, bladder, and prostate, using the technology in order to minimize patient morbidity and improve recovery. Urinary stone disease affects a large number of people both in the United States and throughout the world. Stones can be caused by a range of medical and anatomic problems and often requires surgical intervention for management. Treatment of stones within the urinary tract using endoscopes and instruments comprises a large portion of the endourology practice, where problems are addressed from within the body. Using these tools urologists have been able to treat stones located within the kidney, ureter, and bladder using endourologic techniques. In addition, other problems of the urinary tract, such as blockages, can be treated in a similar fashion. Like much like the endoscopes used in ENT, there is a need to have additional channels in which others tools and accessories can be used to treat more complicated surgeries such as prostate cancer, ureteropelvic junction (UPJ) obstruction, bladder and kidney cancer and vesicoureteral reflux.

Minimally invasive surgical options are available to many people facing urologic surgery. The most common is laparoscopy, which uses small incisions. Laparoscopy can be very effective for many routine procedures, but limitations of this technology prevent its use for more complex urologic surgeries.

A new category of surgery, Robotic Surgery utilizing the da Vinci® Surgical System made by Intuitive Surgical (Sunnyvale, Calif.) and the Sensei System made by Hansen Medical (Mountain View, Calif.). The da Vinci® Surgical System is being used by surgeons for prostatectomy, bladder reconstruction, gynecologic oncology, hysterectomies, myomectomies, lymph node biopsies, uterine fibroid removal, pelvic prolapse, kidney transplant, bariatric surgery, coronary artery bypass grafting, hysterectomy, cholecystectomy, and mitral valve repair. It is a minimally invasive approach, using surgical and robotics technologies. This includes prostatectomy, where the target site is not only tightly confined but also surrounded by nerves affecting urinary control and sexual function. Much like the laparoscopic, endoscopic and bronchoscope procedures, robotic surgeries require multiple ports and in which tools and accessories are used to perform the procedure.

Shoulder arthroscopy is surgery that uses a tiny camera to examine and facilitate minimally invasive repair. Surgeons complete rotator cuff repairs where the edges of the muscles are approximated and the tendon is attached to the bone often with sutures, suture anchors or a combination. In situations where this diseased tissue that is no longer functional, debridement or tissue removal is completed through the same small incision under the guidance of the arthroscope. The surgeons also frequently treat shoulder instability and use small tools designed to work through similar small incisions in the skin. Tools include shavers, aspirators, bites, cutting tools, cinching tools and the like. It is often difficult to precisely aim some of these tools towards the target anatomy to complete the procedure. As such it would be highly desirable to have a means that could be used steer or aim the tools in the desired trajectory better position them for usage during the procedure. The ability to customize and move to alternative locations to redirect tools would also be of great utility and provide new capability to the surgeons in these procedures.

Like the shoulder, knee arthroscopy is completed by placement of a small camera through a small incision about the knee. Many knee problems can then be intervened using minimally invasive tools positioned through one or more small incisions placed near the camera access site. For example, these problems include repair or removal of a torn meniscus (i.e. the cartilage that cushions the space between the bones in the knee), repair or reconstruction of a torn or damaged anterior cruciate ligament, repair of knee bone fractures and the like. As with arthroscopic shoulder surgeries, placement of tools and instruments into the field via small incision access points often limits the capability of the surgeon to effectively reach, position or aim these devices in the desired trajectory. It would therefore be highly desirable if a catheter system could be used wherein the tip of the catheter could be more precisely aimed or directed to the target anatomy once access through the skin was completed. This customization of the catheter tip would ideally occur reliably via some means which did require visual confirmation through the scope or that could occur through some means that is positioned out of the line-of-sight of the scope.

Minimally invasive ankle surgery is accomplished similarly to the knee and shoulder arthroscopic procedures. Upon access through an incision in the skin, the camera is positioned to visualize the target anatomy and a second incision is then made nearby the scope's access point to facilitate placement of specialty tools designed for ankle procedures. Typical procedures are completed to treat ankle arthritis, anterior ankle impingement, unstable ankle, lateral ligament reconstruction, ankle pain following fracture, loose bodies within the ankle, osteochondral defects of the talus, and the like. The procedural flexibility provides some means to redirect, steer or aim the tools in alternate trajectories would be of great utility in these procedures as well. Further, it would ease the procedural burden if the shape transformation could be done via a reliable mechanism or indicator system that precludes the necessity to confirm the change visually.

Chronic rhinosinusitis or inflammation of the nose and paranasal sinuses, is a condition that reportedly affects 37 million people each year accounting for as many as 22 million office visits and 250,000 emergency room visits per year in the United States. Inflammation of the paranasal ostia restricts the natural drainage of mucous from the sinus cavity through mucocilliary clearance resulting in chronic infections within the sinus cavity. Symptoms of chronic rhinosinusitis include extreme pain, pressure, congestion, and difficulty breathing. The first line of treatment for chronic rhinosinusitis is medical therapy including the administration of medications such as antibiotics and anti-inflammatory agents such as steroids. Patients that are unresponsive or refractory to this medical therapy typically are considered for surgical intervention to help relieve these symptoms of the condition. Functional endoscopic sinus surgery (FESS) is currently the most common type of surgery used to treat chronic sinusitis by remodeling the sinus anatomy. In a typical FESS procedure, an endoscope is inserted into the nose or nostril often along with a variety of surgical instruments. These have traditionally included but are not limited to the following tools: applicators, chisels, curettes, elevators, forceps, gouges, hooks, knives, saws, mallets, morselizers, needle holders, osteotomes, ostium seekers, probes, punches, backbiters, rasps, retractors, rongeurs, scissors, snares, specula, suction canulae and trocars. These instruments are then used to cut tissue and/or bone, cauterize, suction, etc. FESS, which was developed as an alternative to open surgical incisions and procedures, encompasses the use of an endoscope along with the listed tools to minimize patient trauma. In these procedures, it would be highly desirable to be able to direct or steer or aim the tools more precisely in the direction of the target tissue or anatomy and it would further be advantageous if this could be accomplished in a reliable manner without the need for confirming the change in the catheter tip visually.

There is also a school of thought that preservation of mucosal tissue during FESS procedures is valuable to long term clinical outcomes. In this regard, balloon dilatation of the sinuses has recently been introduced to the market by a number of companies as a minimally invasive approach to FESS. In this technique, the sinus surgeon places an endoscope and a guide catheter in the patient's sinus cavity usually via insertion through the nostrils. The surgeon advances a guide catheter with a preset geometry into a position that is close to the target sinus ostium after which a guidewire is introduced into the target sinus cavity. A dilatation catheter is then loaded over the guidewire and advanced until the dilatation mechanism is in the sinus ostium after which the sinus ostium and outflow tract are expanded using high pressure. In doing this sequence of steps, the boney structures underlying the sinus ostium that contact the dilatation catheter are remodeled and often fractured while preserving or sparing the overlying mucosa.

While an improvement over prior practice, these types of systems typically employ multiple working devices (e.g. an endoscope, sinus seeker, guide catheter, guidewire, dilatation catheter, etc.). The management and effective (often simultaneous) operation of these multiple tools in the surgical procedural setting can present a significant challenge to the surgeon. For example, at points in the procedure the surgeon is required to hold the endoscope in place in the sinus cavity while maintaining the position of the guide catheter and simultaneously advancing and directing the dilatation catheter into or through the target sinus ostium. Successful use of these often distinct, uncoupled devices requires intensive training and skill and the requirement that many of these items be used concurrently can limit the physician's ability to provide the desired level of precision and accuracy. The level of complexity of such procedures is exacerbated when multiple sinus ostia are treated in the setting of a single procedure. In such cases, multiple guide catheters with varying tip angles or malleable formable tips and other apparatus are often required to successfully locate and cannulate the targeted sinus passageways. Due to patient to patient variation in sinus anatomy, the surgeon is required to stock each of these variations of the guide catheters in their disposable equipment inventories occupying valuable space in the operating room or healthcare facility and adding an economic burden to maintain these stock inventories for daily procedural use.

Recently Entellus Medical (Minnesota, USA) introduced the XprESS Multi-Sinus Dilation Tool to address some of these shortcomings The XprESS tool is a combination device comprised of a ball-tipped malleable shaft with a thru lumen that is intended to generally mimic the concept of the traditional sinus seeker used by surgeons. XprESS augments this sinus seeker-like component with a dilatation balloon catheter that is coaxially positioned over the outside wall of the malleable shaft. The hub of the device allows the surgeon to apply a suction pressure to the distal tip of the malleable shaft, if desired, and the thru-lumen of the malleable shaft can be used to position a guidewire too confirm device location in the sinus anatomy if necessary. The hub also has a luer connector to allow attachment of a syringe to control inflation and deflation of the balloon. Finally, the hub includes a balloon slide mechanism, which is intended to allow positioning of the balloon over the malleable shaft after it has been positioned at the desired sinus target. The malleable shaft is constructed from a material that allows it to be shaped by the surgeon in the field to a fixed geometry that the surgeon believes will be adequate to access the desired anatomy of the patient. While this innovation may eliminate the need for multiple fixed tip angle guide catheters, the act of shaping or reshaping the malleable shaft must necessarily take place outside of the sinus and requiring the surgeon to use a trial-and-error approach to gaining successful access since the shape cannot be modified while inside the patient in proximity to the target anatomy. Also, the physician has to estimate the tip angles and physically shape the tip lending to less precision and extended procedures times. Further, the ball shaped distal most tip of the malleable shaft may be traumatic to the mucosa and possibly bone while the shaft segment is positioned using a sinus seeker-like technique in advance of balloon insertion. It would be desirable to have means to reshape the catheter or guide device once inside the body of the patient and furthermore it would be advantageous to be able to reliably enable the shape change with a mechanism that does require visual confirmation of the change at the tip. It is clear that these advantages would also apply to positioning other interventional tools and implants (included stents and drug delivery stents, spacers, materials, & devices).

In summary, these various examples demonstrate the plethora of medical procedures that exist and are being developed that could benefit from improved catheter means that could make access of target anatomy simpler, faster or reliable. More specifically, many of these procedures require treatment of multiple sites in the same setting. The present invention addresses these needs.

### RELEVANT LITERATURE

U.S. Pat. No. 7,670,282; U.S. patent application Ser. Nos. 12/561,147, 61/352,244 and 61/366,676.

### SUMMARY

Among the various embodiments, objects and features of the present invention may generally be noted a steerable guide system which simplifies and eases access to and optionally treatment of one or more target anatomies in various medical procedures thereby reducing procedure time, equipment burden, and associated costs.

More specifically, one object of the present invention is to enable single and/or multiple diagnostic and/or interventional treatments of different target sites without the need for device exchanges.

A second object of the invention is to allow physicians/users to modify or transform the shape of the distal segment or tip of a guide device to a desired geometry (tip angle and rotational position) both ex vivo and/or in vivo (i.e. inside and/or outside the human or animal body) using feedback mechanisms or indicators at the proximal end of the system that precludes the need for any visualization means to confirm the shape change at the distal segment or tip.

A third object of the invention is to allow physicians/users to modify or transform the shape of the distal segment or tip of a guide device to a predetermined geometry (tip angle and rotational position) both ex vivo and/or in vivo (i.e. inside and/or outside the human or animal body using a feedback mechanisms or indicators at the proximal end of the system that precludes the need for any visualization means to confirm the shape change at the distal segment or tip.

A fourth object of the invention is to allow the physicians a means to aim & maintain diagnostic and interventional tools and instruments in the desired trajectory.

A fifth object of the invention is to reduce radiation exposure to users of the system in medical procedures that require visualization means to that emit radiation like fluoroscopy and the like.

The various embodiments of the subject invention included herein provide devices, systems and methods for improving access to body cavities, lumens, or ostia (especially narrowed ostia). The scope of the inventions in this specification includes methods and devices that reduce the number of devices and materials required for the treatment, expedite procedure time and improve ease of use in procedures that treat restrictions in the human and animal body. The various embodiments could also be used in body cavities or lumens or openings wherein body cavities are defined to be any open and/or hollow and/or potential space in the body of a subject and lumens are defined to be the interior space of any conduit or tube structure in the body of a subject and openings are defined to be passages (restricted or otherwise) that describe the entrance or exit of a conduit, and ostia are defined as small openings or passages into a body organ or conduit.

In accordance with one embodiment, a steerable elongate guide system is formed by a series of components including a transport member having a straight segment with a pre-formed shape at its distal or terminal end. The transport member can alternatively be referred to as a pre-shaped or pre-formed guide, may comprise a lumen or lumens extending the length of the transport member, may comprise an elongate member without a lumen, or may comprise an elongate member with an internal cavity or cavities The internal cavities of the transport member may be in communication with the external surface of the transport member. The transport member may be housed within a substantially rigid, elongate cannula or tube slidably disposed coaxially over the transport member. Both the transport member and cannula may include hubs for attachment to other standard equipment like suctions lines, syringes etc. These hubs could feature standard luer connections. In this embodiment, when the rigid cannula covers the pre-formed shape segment of the transport member, the pre-formed shape assumes a constrained configuration that generally follows the inner geometry of the substantially rigid cannula. When the cannula is retracted proximally with respect to the transport member, the transport member is sequentially exposed and resumes a portion or all of its performed shape. The full pre-formed shape is achieved when the rigid cannula is fully retracted onto the straight segment of the transport member. Alternatively, the transport member could be moved proximally with respect to the substantially rigid cannula to achieve the same result.

In another embodiment, a steerable elongate guide system is formed by a series of components including a transport member having a straight segment with a pre-formed shape at its distal or terminal end. This transport member may house a substantially rigid, elongate cannula or tube slidably disposed coaxially within the transport member. When the distal end of the cannula is flush with or extending past the distal end of the transport member, the transport member would assume a configuration that mimics the geometry of the underlying cannula. When the cannula is retracted proximally with respect to the transport member, the transport member sequentially assumes a portion or all of its performed shape. The full pre-formed shape is achieved when the rigid cannula is fully retracted into the straight segment of the transport member. In another embodiment, the transport member could be moved proximally with respect to the substantially rigid cannula to achieve the same result.

In any of the aforementioned embodiments, one or more retaining members may be positioned between the transport member and the cannula to prevent relative motion of the two components. These retaining members could be incorporated into one or both of the hubs of the transport member or cannula. Alternatively, the retaining members could be an additional component or components that could be removed or deactivated to enable relative motion between the transport member and cannula. The cannula and/or the transport member could feature single or multiple lumens which could be used for the transport and delivery of diagnostic and interventional tools to an anatomical site in a human or animal, infusion of medications, aspiration or suction or the like, illumination of the target anatomy and surroundings, imaging and visualization etc. These lumens can be of the same dimension from proximal to distal ends or alternatively can taper or expand along the length of the transport member and/or cannula. A retaining member such as an o-ring, clip, Touhy-Borst valve, etc. could be used to retain any contents placed within the lumen of the transport member. For example, a balloon catheter may be placed into the transport member lumen prior to insertion or delivery into a human or animal subject. The transport member and/or cannula may be fabricated from composites, homogenous metallic and/or polymeric materials, braided constructions and the like. The transport member could be constructed from materials that effectively transmit torque force, allowing one to grasp and rotate the transport member housed within the cannula to move or aim the preformed shape into the desired trajectory. The transport member could rotate with respect to the cannula or the two components could rotate as a unit if desired. The tip of the cannula and/or the transport member could be constructed from materials that make them atraumatic and flexible to minimize the potential for damage to the anatomy during handling and maneuvers. The materials used for any of the system components could be rendered radiopaque or radiolucent as desired. Also, lubricious coatings or other methods of reducing friction may be employed in conjunction with the system and sub-components of the invention.

Any of the inventions or the embodiments of the inventions described above may be coupled for use in conjunction with visualization devices like endoscopes. The steerable elongate guide system could be mechanically attached or clipped to the endoscope to minimize the number of independent devices that the operator or surgeon must control or handle during a surgical procedure. Alternatively, the steerable elongate guide system may comprise a handle or hub extension that allows the system to be held adjacent to the endoscope using a single hand freeing the other hand for manipulation of the system, adjustment of the endoscope, insertion or removal of devices through the system or the like. The handle or hub extension of this embodiment could be rigid or malleable to permit the handle to change in any orientation or plane relative to the system.

In accordance with still another aspect of the invention, a method is provided for access and multiple dilations (e.g. in the paranasal sinuses) of a human or animal subject. The method includes inserting a steerable elongate guide system into the nose of a human or animal subject and positioning the system near the target sinus for which treatment is required. The steering and/or rotational (e.g. through torque transmission) features of the invention are employed to direct or aim the tip of the elongate guide member in the desired trajectory (e.g. generally towards the sinus ostium, around the uncinate process etc). Inserting and/or advancing a dilation device such as the Relieva Solo Pro™ Sinus Balloon Catheter (Acclarent), the Relieva Solo™ Sinus Balloon Catheter (Acclarent), or the balloon dilation device described in co-pending U.S. Pat. App. No. 61/352,244 herein incorporated in full by reference, and the like out of the steerable guide system and into or through the specific target anatomy (e.g. sinus ostium that requires treatment) is followed by expansion of the dilation device to remodel the sinus ostium and/or sinus outflow tract. The dilation device may then be returned to its unexpanded state and retracted into the transport member of the steerable guide system. The steerable guide system may then be re-positioned to target a different part of the anatomy.

Alternatively, a guidewire may be introduced into the lumen of the steerable elongate guide system after the steering and/or rotational features of the invention have been employed to position the tip of the steerable elongate guide system in the desired trajectory. The guidewire may then be advanced into or through the target sinus ostium, after which the dilation device may be inserted into the lumen of the elongate guide system and tracked over the guidewire to the desired position within the target sinus ostium. The dilation device may then be activated to remodel the sinus ostium and/or sinus outflow tract. In some cases, the guidewire may be removed from the lumen of the dilation device prior to activation of the device. The dilation device may then be returned to its unexpanded state and retracted into the transport member of the steerable elongate guide system. The steerable elongate guide system may then be re-positioned to target a different part of the anatomy.

In a second example, the diameter of the steerable elongate guide system is sized to fit within the lumen of an over-the-wire or rapid exchange dilation device. In this example, a method is provided for access and multiple dilations (e.g. in the paranasal sinuses) of a subject. The method includes preparing the steerable elongate guide system and dilation device by inserting the cannula and transport member of the steerable elongate guide system through the lumen of the dilation device such that the distal end of the steerable elongate guide system extends beyond the distal end of the dilation device. The distal portion of the steerable elongate guide system is inserted into the nose of a human or animal subject and positioned near the target sinus for which treatment is required. The steering and/or rotational (i.e. through torque transmission) features of the invention are employed to direct or aim the tip of the elongate guide member in the desired trajectory (e.g. generally towards the sinus ostium, around the uncinate process etc). An appropriately sized guidewire is introduced into the lumen of the steerable elongate guide system and advanced into or through the target sinus ostium. The dilation device is then advanced distally over the steerable elongate guide system and the underlying guidewire until the working segment of the dilation device is within the target sinus ostium, after which the dilation device is engaged to expand and remodel the sinus ostium and/or sinus outflow tract. In some cases, the guidewire may be removed from the lumen of the dilation device prior to activation of the device. The dilation device may then be returned to its unexpanded state and retracted proximally over the transport member of the steerable elongate guide system. The guidewire may then be retracted into the lumen of the steerable elongate guide system and the steerable elongate guide system may then be re-positioned to target a different part of the anatomy to repeat these procedural steps.

In a third example, the diameter of the steerable elongate guide system is sized to fit within the lumen of an over-the-wire or rapid exchange dilation device. A method is provided for access and multiple dilations (e.g. in the paranasal sinuses) of a subject. The method includes preparing the steerable elongate guide system and dilation device by inserting the cannula and transport member of the steerable elongate guide system through the lumen of the dilation device such that the distal end of the steerable elongate guide system extends beyond the distal end of the dilation device, wherein the transport member comprises a guidewire, coil, or similar structure. The distal portion of the steerable elongate guide system is inserted into the nose of a human or animal subject and positioned near the target sinus for which treatment is required. The steering and/or rotational (i.e. through torque transmission) features of the invention are employed to direct or aim the tip of the steerable elongate guide member in the desired trajectory (e.g. generally towards the sinus ostium, around the uncinate process etc). The distal end of the steerable elongate guide system is advanced into and/or through the target sinus ostium. The dilation device is then advanced distally over the steerable elongate guide system until the working segment of the dilation device is within the target sinus ostium, after which the dilation device is engaged to expand and remodel the sinus ostium and/or sinus outflow tract. The dilation device may then be returned to its unexpanded state and retracted proximally over the transport member of the steerable elongate guide system. The steerable elongate guide system may then be retracted from the treated sinus ostium and re-positioned to target a different part of the anatomy to repeat these procedural steps.

In a fourth example, the invention may comprise an over-the-wire dilation device irreversibly mounted on a steerable elongate guide system. For example, the steerable elongate guide system may comprise a cannula and transport member that can translate and rotate relative to each other. The transport tube in this example comprises a shaped distal segment and resides within a substantially rigid cannula. The over-the-wire or rapid exchange dilation device may be an expandable balloon wherein the balloon lumen is formed from the outer surface of the substantially rigid cannula and the inner surface of a balloon shaft. The balloon shaft in this example is an elongate member with a lumen running from the proximal to distal ends that is mounted coaxially over the cannula. A method is provided for access and multiple dilations (e.g. in the paranasal sinuses) of a subject. The method includes inserting the combined guide/dilation system into the nose of a human or animal subject and positioning the distal end of the combined guide/dilatation system near the target lumen for which treatment is required. The steering and/or rotational (i.e. through torque transmission) features of the invention are employed to direct or aim the tip of the transport member in the desired trajectory (e.g. generally towards the sinus ostium, around the uncinate process, towards a side-branching artery, traversing a rotator cuff, etc). An appropriately sized guidewire is inserted through the lumen of the transport member and through the target body lumen and/or ostium. The combined guide/dilation device is then advanced distally over the stationary guidewire until the working segment of the dilation device is within the target body lumen and/or ostium, after which the dilation component of the combined guide/dilation device is engaged to expand the target body lumen and/or ostium. The dilation component of the combined guide/dilation device may then be returned to its unexpanded state and retracted proximally over the guidewire and out of the target body lumen and/or ostium after which it may be re-positioned to target a different part of the anatomy to repeat these procedural steps.

In an alternative embodiment, the combined guide/dilation device may comprise a steerable wire guide as the transport member. In this example, the coaxial arrangement of cannula and transport member is replaced with a single elongate member that has at least one lumen extending from its proximal end to its distal end. The distal end of a wire or other component capable of transmitting a tensile or compressive load is fixed to the distal end of the elongate member. The proximal end of the force-transmitting component is available to the user to place a compressive or tensile load on the distal tip of the elongate tube. The components may be housed within a casing or shell that permits ease of handling of the steerable elongate guide system. The force-transmitting component may run through a lumen of the transport member, in the wall of the transport member, along the outer surface of the transport member or a combination thereof among other configurations. The application of a force on the proximal end of the force-transmitting member will curve the distal end of the transport member in a pre-determined direction. The distal end of the transport member may be modified to aid in the formation of a desired curve or shape. This may be accomplished through methods known in the art including, but not limited to laser cutting, altering material characteristics such as elasticity, or altering physical dimensions such as inner diameter, outer diameter, and or wall thickness among others. The method of use of this embodiment of the invention is identical to that described above.

In a fifth example, the transport member may alternatively comprise a coiled guidewire, a shaped mandrel made from materials well known in the art (e.g. nylon, PET, Pebax, nitinol, stainless steel, polyurethane, etc.) or other configurations known in the art. For example, the elongate member may comprise a standard coiled guidewire with a pre-formed shape in the distal section of the guidewire. The preformed shape may be such that the distal tip of the guidewire maintains a position ranging from 0 degrees to 180 degrees from the longitudinal axis of the guidewire. In this example, the rigid cannula covers the pre-formed shape segment of the coiled guidewire and forces the coiled guidewire to assume a constrained configuration that generally follows the inner geometry of the substantially rigid cannula. When the cannula is retracted proximally with respect to the coiled guidewire, the distal section of the guidewire is sequentially exposed and resumes a portion or all of its performed shape. The full pre-formed shape is achieved when the rigid cannula is fully retracted onto the straight segment of the guidewire. Alternatively, the guidewire could be moved proximally with respect to the substantially rigid cannula to achieve the same result. Though this example references a coiled guidewire as a non-limiting illustration of the embodiment; other materials and configurations are easily accessible to those of skill in the art.

A method is provided for access and multiple dilations (e.g. in the paranasal sinuses) of a subject using the invention of this example. The method includes preparing the steerable elongate guide system and dilation device by inserting the cannula and transport member of the steerable elongate guide system through the guidewire lumen of the dilation device such that the distal end of the steerable elongate guide system extends beyond the distal end of the dilation device. The distal portion of the steerable elongate guide system is inserted into a human or animal subject and positioned near the target lumen for which treatment is required. The steering and/or rotational (i.e. through torque transmission) features of the invention are employed to direct or aim the tip of the elongate guide member in the desired trajectory (e.g. generally towards the sinus ostium, around the uncinate process, towards a side-branching artery, traversing a rotator cuff, etc). The distal end of the steerable elongate guide system is advanced into and/or through the target body lumen and/or ostium. The dilation device is then advanced distally over the steerable elongate guide system until the working segment of the dilation device is within the target body lumen and/or ostium, after which the dilation device is engaged to expand and treat the target lumen. The dilation device may then be returned to its unexpanded state and retracted proximally over the transport member of the steerable elongate guide system. The steerable elongate guide system and dilation device may then be retracted from the treated body lumen and/or ostium and re-positioned to target a different part of the anatomy to repeat these procedural steps.

In a sixth example, a steerable elongate guide system is formed by a series of components including an elongate coiled wire that terminates in an atraumatic (e.g. hemispherical, spherical, etc.) distal tip. The proximal end of the elongate coiled wire is fixed to a relatively rigid member such that the lumen of the elongate coiled wire is in communication with the lumen of the relatively rigid member. The relatively rigid member is housed within a casing or shell that permits ease of handling of the steerable elongate guide system. A relatively stiff mandrel runs through the lumen of the elongate coiled wire and is fixed to the atraumatic tip at the distal end of the mandrel and fixed to the relatively rigid member at the proximal end of the mandrel. A tapered mandrel runs through the lumen of the elongate coiled wire and the relatively rigid member. The distal tip of the tapered mandrel is fixed to the atraumatic tip of the elongate coiled wire. The proximal tip of tapered mandrel is fixed to a slide or actuator that extends through a groove or channel in the casing or shell. Advancing the slide or actuator distally places a compressive load on the tapered mandrel, which in turn imparts a curved shape to the elongate coiled wire. The radius of curvature of the elongate coiled wire and the magnitude of the curvature are can be modified by changing the location and severity of the taper and/or by changing the distance the slide or actuator is advanced. Alternatively, the relatively stiff mandrel may be replaced with a component capable of supporting and transmitting a tensile load. These force-transmitting components may run through the lumen of the elongate coiled wire as described in this example, or they may reside in the wall of the transport member, along the outer surface of the transport member or a combination thereof among other configurations. The method of use of this embodiment of the invention is identical to that described above.

In a seventh example, a steerable balloon catheter is enclosed in a shell or handle that allows the steerable balloon catheter to translate proximally or distally with respect to the shell. A method is provided for using the device of this example to access and/or treat multiple body lumens and/or ostia. The method includes inserting the steerable balloon catheter into a human or animal subject and positioning the distal end of the combined guide/dilatation system near the target lumen for which treatment is required. The steering and/or rotational (i.e. through torque transmission) features of the invention are employed to direct or aim the tip of the transport member in the desired trajectory (e.g. generally towards the sinus ostium, around the uncinate process, towards a side-branching artery or other body lumen, traversing a rotator cuff, etc). An appropriately sized guidewire is inserted through the lumen of the transport member and through the target body lumen and/or ostium. The steering and/or rotational features of the invention may then be optionally returned to their initial state. The combined guide/dilation device is then advanced distally with respect to the shell via a trigger, slide, rack and pinion mechanism, screw drive mechanism, or other means known in the art to provide the desired amount of leverage and to ease operation. The shell may comprise a retaining member known in the art such as but not limited to an o-ring, Touhy-Borst valve, living hinge, iris valve, ball valve, clamp, chuck, or combination thereof that fixes the position of the guidewire with respect to the shell. In this manner the working segment of the dilation device progresses distally with respect to the fixed shell and guidewire until it is within the target body lumen and/or ostium, after which the dilation component of the steerable balloon catheter is engaged to expand the target body lumen and/or ostium. The dilation component of the steerable balloon catheter may then be returned to its unexpanded state and retracted proximally over the guidewire, out of the target body lumen and/or ostium, and returned to its original position within the shell. The guidewire may be retracted into the body of the steerable balloon catheter, after which the device may be re-positioned to target a different part of the anatomy to repeat these procedural steps.

In an alternative embodiment, the steerable balloon catheter system may further comprise a telescoping sheath component that is coaxially arranged over the dilation component of the catheter. In the case of the dilation component comprising an expandable balloon, the telescoping sheath is coaxially disposed over the balloon shaft. The telescoping sheath may be positioned to cover the dilation element prior to activation of the dilation element. The telescoping sheath may add several features to the steerable balloon catheter system including, but not limited to increasing the lubricity of the device, reducing the rigidity of one or more tissue-contacting surfaces of the device, increasing the stiffness of one or more sections of device, providing a pathway for aspiration or sampling or removal of body fluids or tissues, providing a marker that enables use in a given visualization system (fluoroscopy, electromagnetic navigation systems, ultrasound, magnetic navigation systems, computed tomography, ultrasound, and the like), protecting the dilation element during transit to the treatment area further reducing the profile and helping to groom the folded/pleated balloon and combinations thereof. A method is provided for using the device of this example to access and/or treat multiple body lumens and/or ostia. The method includes inserting the steerable balloon catheter system into a human or animal subject and advancing the distal end of the device into a position near the target lumen while the telescoping sheath is in position over the expandable element of the dilation component. The steering and/or rotational (i.e. through torque transmission) features of the invention are then employed to direct or aim the tip of the transport member in the desired trajectory (e.g. generally towards the sinus ostium, around the uncinate process, towards a side-branching artery or other body lumen, traversing a rotator cuff, etc). An appropriately sized guidewire is inserted through the lumen of the transport member and through the target body lumen and/or ostium. The steering and/or rotational features of the invention may then be optionally returned to their initial state. The telescoping sheath is retracted distally to expose the expandable element of the dilation component and the steerable balloon catheter system can then be advanced distally with respect to the shell via a trigger, slide, rack and pinion mechanism, screw drive mechanism, or other means known in the art. The shell may comprise a retaining member known in the art such as but not limited to an o-ring, Touhy-Borst valve, living hinge, iris valve, ball valve, clamp, chuck, or combination thereof that fixes the position of the guidewire with respect to the shell. In this manner, the working segment of the dilation device progresses distally with respect to the fixed shell and guidewire until it is within the target body lumen and/or ostium, after which the dilation component of the steerable balloon catheter system is engaged to expand the target body lumen and/or ostium. The dilation component of the steerable balloon catheter system may then be returned to its unexpanded state and retracted proximally over the guidewire, out of the target body lumen and/or ostium, and returned to its original position within the shell. The telescoping sheath may be advanced distally to cover the expandable element of the dilation component of the device. The guidewire may be retracted into the body of the steerable balloon catheter system, after which the device may be re-positioned to target a different part of the anatomy to repeat these procedural steps.

In an eighth example, a steerable sheath may comprise an elongate member with a lumen extending from the proximal to distal ends of the member. The steerable sheath may further comprise cuts through the wall of the distal portion of the sheath and a wire bonded to the distal end of the sheath. A compressive or tensile load placed on the wire will be transmitted to the distal end of the sheath, causing the distal segment of the sheath to curve in a direction and degree dictated by the magnitude of force placed on the wire and the pattern or design of the cuts (e.g. shape, distribution, alignment, etc.) on the distal section of the sheath. The proximal end of the sheath may be bonded to a hub that facilitates the insertion and stabilization of other components, such as balloon catheters and/or guidewires. The hub may comprise mechanisms including, but not limited to an o-ring, Touhy-Borst valve, living hinge, iris valve, ball valve, clamp, chuck, or combination thereof. A method is provided for using the device of this example to access and/or treat a body lumen and/or ostium. The method includes inserting the steerable sheath into a human or animal subject and advancing the distal end of the device into a position near the target lumen. The steering and/or rotational (i.e. through torque transmission) features of the invention are employed to direct or aim the tip of the transport member in the desired trajectory (e.g. generally towards the sinus ostium, around the uncinate process, towards a side-branching artery, traversing a rotator cuff, etc). A secondary device (e.g. a guidewire, balloon catheter, aspiration tube, etc.) may be inserted through the lumen of the steerable sheath and into or through the target body lumen and/or ostium. At this point the steerable sheath may be removed and the procedure may continue.

Alternatively, the steerable sheath may be integrated in a telescoping manner on another tool such as a guidewire or balloon catheter. For example, a balloon catheter may be introduced into the proximal thru-lumen of the steerable sheath and advanced until the balloon portion of the balloon catheter is located in the distal section of the steerable sheath. The hub of the steerable sheath would act to retain the balloon catheter in position within the steerable sheath. In this configuration, the steerable sheath would act as both a protective covering over the balloon portion of the balloon catheter and a controllably deflectable tip. The steerable sheath may be assembled telescopically over the balloon catheter at the time of use, or alternatively, the steerable sheath/balloon catheter may be integrated and manufactured as a single unit. A method is provided for using the devices of this example to access and/or treat multiple body lumens or ostia. The method includes inserting the integrated balloon catheter/steerable telescoping sheath system into a human or animal subject and advancing the distal end of the device into a position near the target lumen while the balloon catheter is in position within the steerable telescoping sheath such that the expandable element of the dilation component is covered. The steering and/or rotational (i.e. through torque transmission) features of the invention are employed to direct or aim the tip of the steerable telescoping sheath in the desired trajectory (e.g. generally towards the sinus ostium, around the uncinate process, towards a side-branching artery, traversing a rotator cuff, etc). An appropriately sized guidewire is inserted into the balloon catheter and through the target body lumen and/or ostium. The steering and/or rotational features of the invention may then be optionally returned to their initial state. The steerable telescoping sheath is retracted proximally along the shaft of the balloon catheter (i.e. away from the target body lumen or ostium) to expose the expandable element of the dilation component. The integrated balloon catheter and steerable telescoping sheath system is then advanced distally with respect to the wire into and/or through the target body lumen and/or ostium and expanded and contracted to treat the target body lumen and/or ostium. The dilation component of the integrated balloon catheter and steerable telescoping sheath system may then be returned to its unexpanded state the integrated balloon catheter and steerable telescoping sheath system may be retracted proximally over the guidewire and out of the target body lumen and/or ostium. The steerable sheath may be advanced distally to cover the expandable element of the dilation component of the device. The guidewire may be retracted into the body of the integrated balloon catheter and steerable telescoping sheath system after which the device may be re-positioned to target a different part of the anatomy and the procedure steps above completed again to achieve access and treatment. One further iteration of the design comprises enclosing the integrated balloon catheter and steerable telescoping sheath system in a shell or handle that allows the integrated balloon catheter and steerable telescoping sheath system to translate proximally or distally with respect to the shell and optionally comprises means to maintain the general position of the guidewire relative to the shell during this translation.

In any of the aforementioned embodiments of the invention, a control hub may be incorporated into the invention to coordinate the relative displacement and shape of the distal (steerable) end of the disclosed devices. The control hub may comprise features such as indicators or markings that relay the angle and/or rotational orientation of the tip of the transport member to the user, indentations or other forms or shapes that allow for ergonomic handling of the steerable guide system, ports for irrigation and/or aspiration lines, and the like. The control hub may be permanently attached the devices of the invention or it may be a removable component of the devices of the invention. In one aspect of this embodiment of the invention, the control hub may be used to steer the distal end of the guide device into a desired trajectory, position, or location within the target anatomy, then be removed to allow working devices to track over the guide device (e.g. dilation devices). Furthermore, any of the aforementioned embodiments of the invention may comprise a handle and/or hub extension that facilitates the holding and/or use of the devices of the invention. The handle and/or hub extension may be connected to a control hub, shell, or other feature or component of the devices of the invention via an extension that may be malleable, shapeable, non-malleable, non-shapeable or any combination thereof.

The steerable elongate guide system along with a treatment (working) device may be removed from the patient after access and/or treatment of an initial body lumen and/or ostium. Alternatively, the steerable elongate guide system and treatment device may be sequentially inserted, removed, and then reinserted into the patient to facilitate treatment of multiple targets (e.g. in the contra-lateral paranasal sinuses, in the ipsilateral paranasal sinuses, contralateral or ipsilateral peripheral vasculature, etc.).

In another embodiment, the method also includes using the steerable elongate guide system and/or the dilation devices of this invention or a commercially available dilation device or balloon in conjunction with a telescope or endoscope or any other visualization means or methods used in medical procedures. For treatment of restricted lumens (e.g. sinus ostia or outflow tracts), the physicians that treat these diseases may use, for example, an endoscope to help identify surrounding anatomy to then help position the steerable guide system in close proximity to the target tissue, lumen or anatomy.

In yet another embodiment, the method also includes attachment of the steerable elongate guide system previously described to the endoscope prior to insertion into the patient. This may be achieved by a number of means such as but not limited to clipping, adhesives, taping, Velcro, or by using a handle such as been described in U.S. patent application Ser. No. 12/561,147 assigned to Acclarent, Inc. and U.S. Pat. No. 7,670,282 assigned Pneumrx, Inc., both herein incorporated in full by reference.

In another embodiment, the method may include steps in which an aspiration catheter is inserted or advanced to the target sinus before or after the ostium has been expanded to help remove excess body fluids such as blood, mucous or the like. Alternatively, the method may also comprise using cannulas or tubes to deliver saline, medications, therapeutic agents, biologics, delivery of implants etc. Yet another alternative would be to deliver alternate tools to the target anatomy (e.g. a catheter based medication injection system, biopsy tissue removal tissues, lavage etc).

These and other objects, advantages, and features of the invention will become apparent to those persons skilled in the art upon reading the details of the disclosure as more fully described below.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIGS. 1A-1C is a series of cross sectional views of a steerable guide system with an outer cannula.

FIGS. 2A-2C is a series of cross sectional views of a steerable guide system with an inner cannula.

FIGS. 3A-3C is a series of cross sectional views of a steerable guide system an expandable section on the distal portion of the transport member.

FIGS. 4A-4B depict a design for identifying and controlling the shape of the distal tip of the transport member component of the steerable guide system.

FIGS. 5A-5B depict a design for identifying, controlling, and fixing the shape of the distal tip of the transport member component of the steerable guide system through the use of a set screw.

FIGS. 6A-6B depict a design for identifying, controlling, and fixing the shape of the distal tip of the transport member component of the steerable guide system through the use of a friction member.

FIGS. 7A-7B depict a design for identifying, controlling, and fixing the shape of the distal tip of the transport member component of the steerable guide system through the use of a friction member coupled to detents along the transport member.

FIGS. 8A-8B depict a design for identifying, controlling, and fixing the shape of the distal tip of the transport member component of the steerable guide system through the use of a ratchet coupled to detents along the transport member.

FIGS. 9A-9B depict a design for identifying, controlling, and fixing the shape of the distal tip of the transport member component of the steerable guide system through the use of a key/keyway system.

FIGS. 10A-10B depict a design for identifying, controlling, and fixing the shape of the distal tip of the transport member component of the steerable guide system through the use of a threaded transport member and tapped cannula hub.

FIGS. 11A-11B depict a control adaptor design for identifying, controlling, and fixing the shape and rotational orientation of the distal tip of the transport member component of the steerable guide system.

FIG. 12 depicts an assembly of a guidewire, a steerable guide system, and a balloon catheter.

FIG. 13 depicts a cross-sectional view of the assembled guidewire, steerable guide system, and balloon catheter at the proximal section of the balloon.

FIG. 14A depicts a side view of the shell of an embodiment of a steerable balloon catheter.

FIG. 14B depicts a cross-sectional view of an embodiment of a steerable balloon catheter.

FIG. 14C depicts a cross-sectional view of the multi-lumen tubing.

FIG. 14D depicts a cross-sectional view of the distal end of the steerable balloon catheter.

FIG. 14E depicts a cross-sectional view of an embodiment of the steerable balloon catheter comprising a stylet.

FIG. 14F depicts a side view of the shell of an alternative embodiment of a steerable balloon catheter.

FIG. 14G depicts a cross-sectional view of an alternative embodiment of a steerable balloon catheter with a shell.

FIG. 14H depicts a cross-sectional view of an embodiment of a steerable balloon catheter without a shell.

FIG. 14I depicts top and side views of one embodiment of the handle of the steerable balloon catheter.

FIG. 15 depicts an assembly of a steerable guide system comprising a guidewire as a transport member and a balloon catheter.

FIG. 16 depicts a cross-sectional view of the assembled steerable guide system and balloon catheter at the proximal section of the balloon.

FIG. 17A depicts a cross-sectional view of a steerable guidewire comprising a control hub.

FIG. 17B depicts a cross-sectional view of an alternative embodiment of a steerable guidewire comprising a control hub.

FIG. 17C depicts a cross-sectional view of the distal tip of one embodiment of a steerable guidewire.

FIGS. 18A-18B depict cross-sectional views of an embodiment of a steerable guidewire.

FIGS. 19A-19B depict cross-section views of an embodiment of a steerable guide system comprising a cannula and transport member.

FIGS. 20A-20B depict cross sectional views of an embodiment of a steerable guide system comprising a pull wire.

FIGS. 21A-21B depict cross sectional views of an embodiment of a sheath with and without an aspiration port.

FIG. 22A depicts a cross sectional view of a embodiment of a steerable balloon catheter comprising an internal pullwire.

FIG. 22B depicts a cross sectional view of a embodiment of a steerable balloon catheter comprising an external pullwire.

FIG. 22C depicts a cross sectional view of a embodiment of a steerable balloon catheter comprising a pullwire that traverses the distal inner wall of the balloon catheter.

FIG. 23 depicts a cross-sectional view of an integrated steerable balloon catheter and a telescoping sheath.

FIG. 24A depicts a cross-sectional view of one embodiment of a steerable sheath.

FIG. 24B depicts a cross-sectional view of the distal tip of one embodiment of a steerable sheath.

FIGS. 25A-25B depict cross-sectional views of an embodiment of an integrated balloon catheter and a steerable telescoping sheath system.

FIG. 26A-26B depict cross-sectional views of an embodiment of an integrated balloon catheter and a steerable telescoping sheath system comprising a shell.

FIG. 27 is a flowchart illustrating a method of use for the devices described in FIGS. 1-12 and 24.

FIG. 28 is a flowchart illustrating an alternative method of use for the devices described in FIGS. 1-12 and 24.

FIG. 29 is a flowchart illustrating a method of use for the devices described in FIG. 14.

FIG. 30 is a flowchart illustrating a method of use for the devices described in FIG. 15.

FIG. 31 is a flowchart illustrating a method of use for the devices described in FIGS. 19 and 20.

FIG. 32 is a flowchart illustrating a method of use for the devices described in FIG. 23.

FIG. 33 is a flowchart illustrating a method of use for the devices described in FIGS. 25 and 26.

FIG. 34 is a flowchart illustrating a method of use for the devices described in FIGS. 25 and 26.

### DETAILED DESCRIPTION

Before the present invention is described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

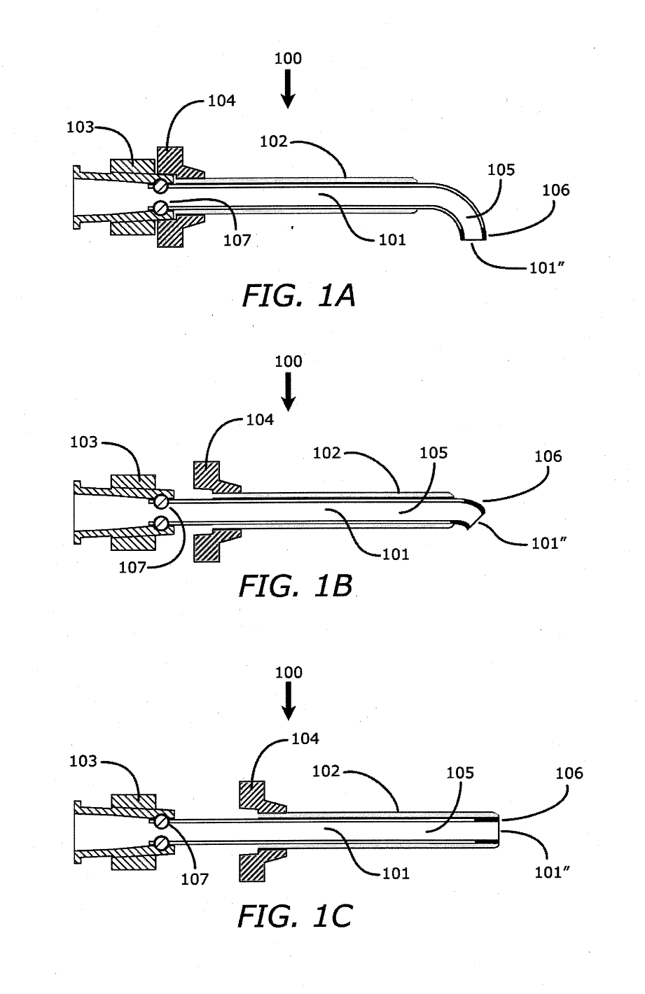

FIGS. 1A-1C provides cross sectional views of one embodiment of the steerable guide system of the invention 100 with delineation of the system components. In this figure, system components include transport member 101, cannula member 102, transport member hub 103, and cannula hub 104. The transport member components 101, 103 could be comprised of a member with a proximal and distal end 101″ with a continuous lumen therethrough. The distal segment of the transport member, 105, could be pre-formed in a desired geometric configuration. For example, the substantially distal segment of the transport member 105 may be pre-formed to position the distal tip 101″ in a generally orthogonal or ninety (90) degree orientation with respect to the straight segment of transport member 101 (proximal to the pre-formed segment). Unconstrained, the distal tip 101″ of the transport member's distal segment 105 would remain at its generally orthogonal or ninety (90) degree position with respect to the proximal segment of the transport member 101. The transport member 101 is shown connected to the transport member hub 103. The transport member hub 103 may be a standard fitting (e.g. luer connection) that allows easy attachment of syringes, extension tubes or lines and other equipment known in the art. Hub 103 could also include or be attached to a manifold (not shown) that allows multiple items to be connected to the proximal end of the transport member through side ports. This could be desirable when the inside lumen of transport member 101 is reserved as working channel for instruments, but it is desirable to use a side port attached to or integrated with hub 103 to aspirate simultaneously. The side port could also facilitate injection of fluids for lavage or the application of medications and the like. The transport member 101 could be constructed from semi-rigid to flexible plastics, polymers, metals and composites including braided tubing configurations well known in the art. For example, transport member 101 could be made from the following non-limiting list of materials: Pebax, nylon, urethane, silicone rubber, latex, polyester, Teflon, Delrin, PEEK, stainless steel, nitinol, platinum etc. Permutations of these materials could also be envisioned. The preformed shape could be achieved through a number of processes such as heat setting, molding, shape memory applications with or without nitinol etc.

FIGS. 1A-1C also highlights a section of the of the transport member, 106. Segment 106 of the transport member 101 in this embodiment could be comprised of or fabricated from a radiopaque agent or other visualization enhancing materials including, but not limited to barium sulfate, tantalum, platinum, gold, platinum/iridium composites, or the like to render it visible under x-rays, fluoroscopy, CT or ultrasound, or the like, and could also include colorants to enable easier direct visualization via endoscopy. Segment 106 may be located at the most distal tip 101″ of the transport member as shown in FIGS. 1A-1C, or alternatively segment 106 may be located at any position along transport member 101. Furthermore, segment 106 may be repeated multiple times along the length of transport member 101 to provide multiple markers for visualization under x-rays, fluoroscopy, computed tomography, ultrasound, direct visualization, infrared modalities, electromagnetic positioning systems or the like.

FIGS. 1A-1C depicts a retaining member 107 that acts to hold the position of any tool inserted in transport member 101 after the physician operator has released the tool. For example, FIGS. 1A-1C shows the retaining member 107 as an o-ring located on the proximal portion of transport member 101. The o-ring 107 would apply enough friction to the shaft or outer surface of a tool, such as a guidewire, balloon catheter, aspiration tool, surgical instrument, or the like, to fix the tool with respect to transport member 101 after insertion and placement of the tool through the lumen of transport member 101. While depicted as an o-ring in FIGS. 1A-1C, retaining member 107 could be any design, component, or feature known in the art that can act to fix a tool with respect to transport member 101. This includes but is not limited to Touhy-Borst valves, clips, detents, lumen narrowing, springs, levers, living hinges, irises, and the like. Retaining member 107 may also be located at any position within transport member 101 or transport member hub 103. Furthermore, multiple retaining members 107 of varied designs may be incorporated into steerable guide system 100.

The cannula member 102 represents a substantially rigid component of the system that also is compromised of a proximal and distal end with a continuous lumen therethrough. Cannula member 102 could have a hub, 104, at its proximal end as shown in FIGS. 1A-1C. As with the transport member hub 103, cannula hub 104 could be used for connection to other devices or components to achieve functional outcomes like aspiration, lavage/irrigation or to apply medications. Hub 104 in the FIGS. 1A-1C also serves as a handle to control the steerable system. Hub 104 could be designed to have appropriate ergonomics to facilitate one-handed, single operator utilization during its use in completing the maneuvers (e.g. advancing or retracting longitudinally or rotation about the longitudinal axis of the cannula 102) of the intended medical procedure. Hubs 103 and 104 could be made from standard metal, plastic, polymer, composite or other materials well known in the art. The process to make these hubs 103 and 104 may include but not limited to well known methods such as injection molding, casting, machining etc. In the embodiment shown in FIGS. 1A-1C, the components 101-104 are arranged with the cannula 102 positioned coaxially over the outer surfaces of the transport member 101. Cannula 102 would be able to move and/or slide in the longitudinal direction both proximally and distally. In the proximal direction, the travel of cannula 102 over transport member 101 would be limited once cannula hub 104 interfered or was retracted to transport member hub 103. In the distal direction, travel would be unconstrained and cannula member 102 could be pushed along the outer wall of transport member 101, until it was completely removed off transport member 101 as a free-standing component. As shown in FIGS. 1A-1C, as cannula 102 is advanced distally it captures preformed shape 105 within its lumen. In doing so, the pre-shaped segment of transport member 105 assumes a shape that generally mimics the geometry of cannula 102. Cannula 102 could be of an overall length that would be less than the overall length of transport member 101. The ideal length for cannula 102 would be one where hub 104 is always in comfortable proximity to the surgeon operator's hands outside the patient. It would also be ideal if cannula 102 could slide proximally and distally over adequate length to steer the distal tip of the transport member 101″ through its range of motion allowing transformation of the transport member 101 from a substantially straight configuration when constrained by cannula 102 to its pre-formed geometry as it is unconstrained.

A handle and/or hub extension (not shown) could be located on the proximal end of the steerable guide system shown in FIG. 1A to 1C. The handle and/or hub extension would allow the user to grasp both the steerable guide system and accessory device (e.g. endoscope) in a single hand freeing the other hand for manipulation of the system, adjustment of the endoscope, insertion or removal of devices through the system or the like. The handle or hub extension of this embodiment could be rigid or malleable to permit the handle to change in any orientation or plane relative to the system. As a non limiting example of this embodiment of the invention, the handle may be rigid and pre-shaped or alternatively constructed from malleable materials that allow reforming or reshaping by the operator or surgeon at the point of use. The rigid handles may be made of materials that include, but are not limited to: polycarbonate, Delrin, nylon, ABS, PEEK, Stainless steel, metal alloys, ceramics or the like. The malleable handle embodiments could be made from materials that include, but are not limited to: copper, stainless steel, aluminum, composite materials such as PEBAX tubing with embedded metallic braiding, brass, or the like. The rigid or malleable component of the handle could be fully or partially covered by a material or materials that ease comfort during handling, enhance grip, improve ergonomics or the like. These materials could include, but are not limited to: silicone rubber, polyurethane, latex, vinyl, butyl rubber, acetyl rubber or the like. The shape of the handle in this embodiment of the invention may be any form that permits the effective single handed stabilization of the steerable guide system and at least one accessory component (e.g. the endoscope). For example, the handle may comprise a “U” shape wherein one leg of the “U” projects from the proximal end of the steerable guide system and the free end of the “U” is used to hold, control and/or stabilize the system adjacent to at least one accessory component (e.g. the endoscope). Alternatively, the leg of the “U” shaped handle could be connected or attached to the proximal end of the steerable guide system such that the orientation of the free end may adjusted in any plane relative to the steerable guide system. The leg of the “U” shaped handle could be attached to the proximal end of the steerable guide system and configured to allow it to hinge, swivel, and/or rotate about the steerable guide system.

As another example, the handle may comprise a chain of links that are connected to each other through friction bearing surfaces. Each individual link in the chain is rigid and not malleable; however, the multitude of friction bearing surfaces allows the operator to adjust the orientation of the handle in order to achieve the desired guide position. The amount of friction between each link may be adjusted to attain a desired amount of resistance to motion in the handle as a whole. Higher friction between the links will produce a handle that requires more force to adjust while lower friction between the links will produce a handle that requires less force to adjust. Furthermore, the amount of friction between individual links may be tuned to impart different properties to different components of the chain. For example, a proximal portion of the chain may be comprised of links that are mated through highly frictional surfaces to enable a relatively static segment that facilitates gripping of the handle and an accessory device. The remainder of the chain may be comprised of links that are mated through less frictional surfaces to enable easy adjustment of the steerable guide position. The individual links in the chain may be solid or hollow. If the links are hollow, a further embodiment of the handle may comprise a tensioning cable running through the center of the chain. When relaxed, the cable allows free, unhindered movement of the chain; when tension is placed on the cable, free movement of the chain is inhibited and the handle is locked to stabilize the shape of the handle after the desired guide position is attained. The cable may be activated by a switch, button, or other control mechanism such that the rest state of the device is either locked or free to move (unlocked).

In yet another embodiment of a non-malleable yet shapeable handle, the handle may comprise a tube of continuous wound metal with interconnected and overlapping segments similar to that found in flexible steel conduit. The tubing may comprise one or more layers and have a finish including, but not limited to chrome plating, brass plating, vinyl-clad, copper plating, enamel, baked enamel, braiding and the like.

While the previously described embodiments of the handle use the steerable guide system 100 as a reference design, it should be obvious that the handle may be used in conjunction with any of the embodiments of the steerable guide system disclosed herein.

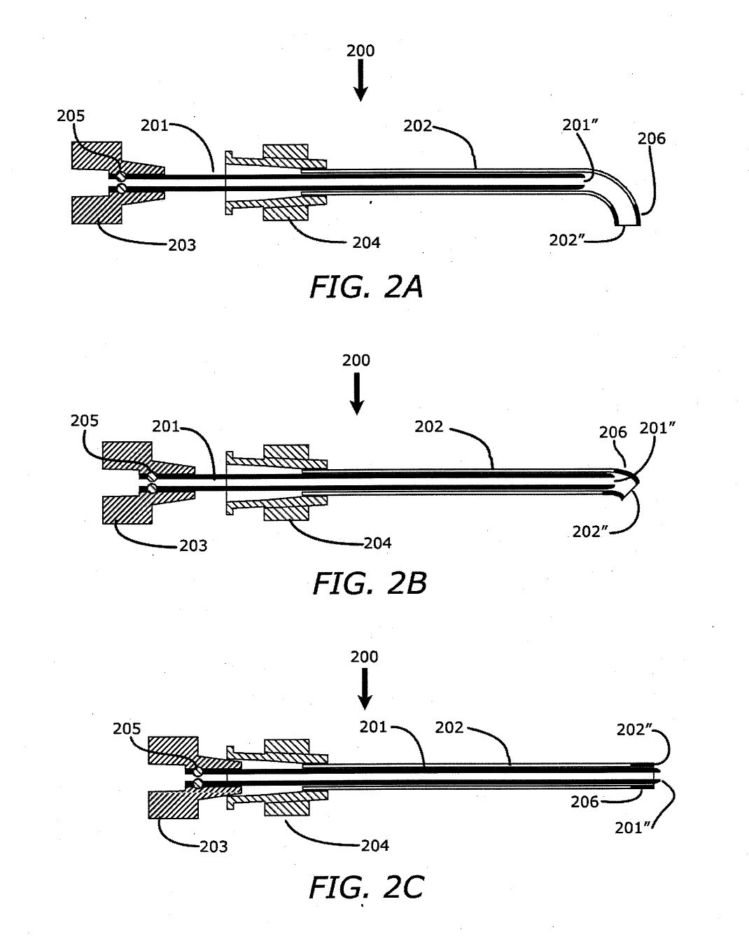

FIGS. 2A-2C provides an alternative embodiment of the steerable guide system 200 of the invention. The general form of the components is similar to those previously described for the embodiment shown in FIGS. 1A-1C. The difference presented by this embodiment is the coaxial configuration of the substantially rigid cannula member 201 inside the lumen of the transport member 202. With this arrangement, cannula member 201 can be retracted proximally, sliding along the inner wall of the transport member 202 until it is a free-standing member. In the distal direction, cannula 201 could be advanced distally along the longitudinal axis of the cannula via its hub 203 until it abuts transport member hub 204. In this embodiment, cannula 201 would be of adequate length wherein the advancement of the rigid cannula 201 would force the preformed shape of transport member 202 to generally mimic the outer geometry of the cannula member 201 when cannula 201 traverses the pre-formed section of the transport member 202. The distal tip of the cannula 201″ may be fabricated from an atraumatic material (e.g. low durometer silicone) and/or in an atraumatic shape (e.g. rounded, conical, etc.) such that is does not damage the internal lumen of transport member 202 during advancement or retraction of cannula 201. When retracted, the pre-formed shape would generally return to the transport member 202 and as discussed in the previous embodiment in FIGS. 1A-1C. This would allow the physician to orient or aim the distal segment of transport member 202 in a desired trajectory within the range of motion of transport member 202 between its preformed and straight segments.

FIGS. 2A-2C also highlights a section of the of the transport member, 206. Segment 206 of the transport member 202 in this embodiment could be comprised of or fabricated from a radiopaque agent or other visualization enhancing materials including, but not limited to barium sulfate, tantalum, platinum, gold, platinum/iridium composites, or the like to render it visible under x-rays, fluoroscopy, CT or ultrasound, or the like, and could also include colorants to enable easier direct visualization via endoscopy. Segment 206 may be located at the most distal tip 202″ of the transport member as shown in FIGS. 2A-2C, or alternatively segment 206 may be located at any position along transport member 202. Furthermore, segment 206 may be repeated multiple times along the length of transport member 101 to provide multiple markers for visualization under x-rays, fluoroscopy, computed tomography, ultrasound, infrared modalities, direct visualization, electromagnetic positioning systems or the like.

FIGS. 2A-2C depicts a retaining member 205 that acts to hold the position of any tool inserted in substantially rigid cannula member 201 after the physician operator has released the tool. For example, FIGS. 2A-2C shows the retaining member 205 as an o-ring located on the proximal portion of substantially rigid cannula member 201. The o-ring 205 would apply enough friction to the shaft or outer surface of a tool, such as a guidewire, balloon catheter, aspiration tool, surgical instrument, or the like, to fix the tool with respect to substantially rigid cannula member 201 after insertion and placement of the tool through the lumen of substantially rigid cannula member 201. While depicted as an o-ring in FIGS. 2A-2C, retaining member 205 could be any design, component, or feature known in the art that can act to fix a tool with respect to substantially rigid cannula member 201. This includes but is not limited to Touhy-Borst valves, clips, detents, lumen narrowing, springs, levers, living hinges, irises, and the like. Retaining member 205 may also be located at any position within substantially rigid cannula member 201 or substantially rigid cannula member hub 203. Furthermore, multiple retaining members 205 of varied designs may be incorporated into steerable guide system 200.

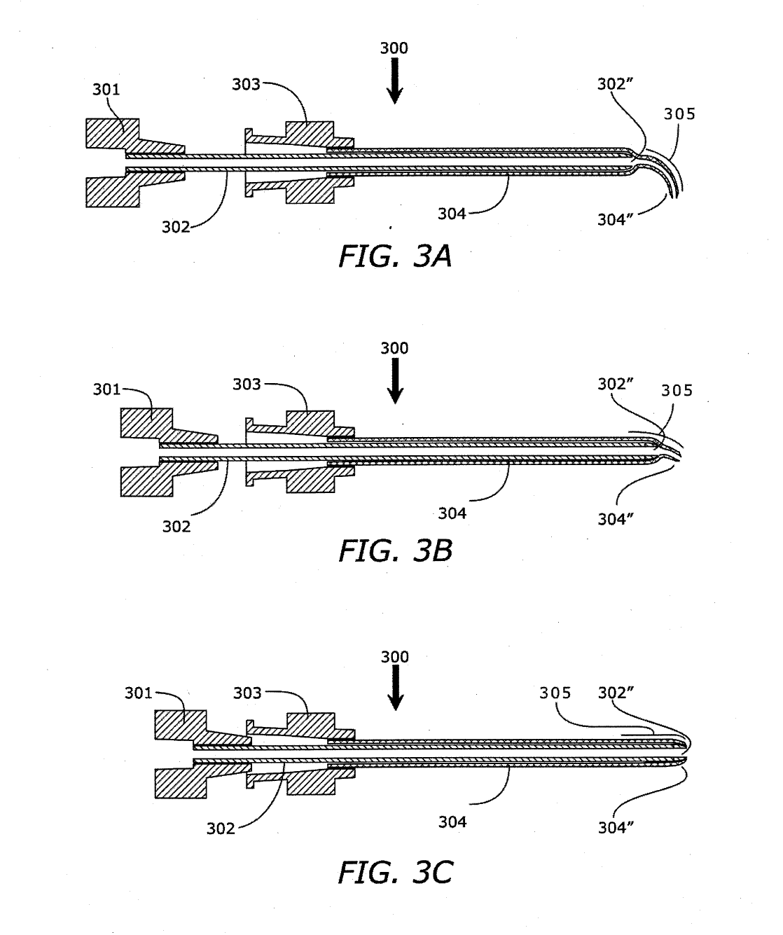

FIGS. 3A-3C depict yet another embodiment of the steerable guide system 300 of the invention comprising cannula hub 301, cannula 302, transport member hub 303, and transport member 304. Distal segment 305 of the transport member 304 has a feature of being normally collapsed in diameter or profile and has compliance characteristics such that the inner dimension would enlarge to conform to the outer dimension of larger devices or instruments passing or being inserted through the collapsed section. The general form of the components and device construction in this embodiment is similar to those previously described for the embodiment shown in FIG. 2A-2C. The difference presented by this embodiment is the configuration of the distal segment 305, which is substantially smaller in diameter relative to the dimensions of the proximal segment of the transport member 304. Preferably, the length of collapsed distal segment 305 may be as long as the entire pre-formed section, including the tip 304″. Alternatively, the collapsed distal segment 305 may be a portion of the pre-formed length or may proximally extend beyond the pre-formed section. The collapsed distal segment 305 may be comprised of a single material or component, or may be a combination of materials or components. For example, the pre-formed collapsed distal segment 305 may be comprised of a component including, but not limited to a weave or braid made of a metallic or non-metallic material (e.g. stainless steel, nylon, nickel titanium, or the like). This pre-formed collapsed distal segment 305 component may be continuous with or may be attached as a separate component from the remaining length of the transport member 304 using known processes including, but not limited to fusing, welding, soldering, crimping, bonding, or the like. As another example, the collapsed distal segment 305 may comprised of a combination of components such as weave or braid (similar to that as described earlier) and an inner liner that allows expansion or contraction or recoil of the collapsed distal segment 305 which may be made from polymeric materials including, but not limited to ePTFE, HDPE, Nylon, and other similar fluoropolymer materials, preferably a material with lubricious property or a material that can be coated to provide lubricity allowing devices to be easily inserted and retracted. Further example includes adding a third component such as an outer liner that allows expansion or contraction or recoil of the collapsed distal segment 305. The collapsed distal segment 305 may have the capability to expand into a profile with diameter larger than that of the remaining length of the transport member 304 to comply and allow fit and operation of devices pre-disposed within the collapsed distal segment 305. Referring to FIG. 3A, the transport member 302 is shown pre-disposed within the lumen of the transport member 304 and the transport member distal end 302″ is positioned proximal of the collapsed distal segment 305. In this example, the collapsed distal segment 305 is pre-shaped to a continuous curve in a single axis/single plane configuration and is in the maximum curve shape. The pre-shaped section/collapsed distal segment 305 can be made such that multi-axis and multi-plane shape can be configured based on the desired need and application (not shown). Turning to FIG. 3B, the cannula 304 depicts a position that is partially advanced distally, thus the transport member distal pre-shaped collapsed segment 305 has transformed into a lesser curve and the direction of the tip 304″ has changed to a lesser angle in relation to the longitudinal axis of the cannula 302. Further, this figure shows that portion of the collapsed segment has expanded and conformed to the size of the cannula 302. Finally turning to FIG. 3C, the transport member 302 is fully advanced in the distal direction such that the distal ends 302″ and 304″ are substantially aligned or flush, thus showing the entire length of the collapsed segment 305 to have expanded and conformed to the size of the cannula 302. Alternatively, the fully inserted position of the cannula 302 may be designed so that the distal ends 302″ and 304″ are offset at some distance from each other.

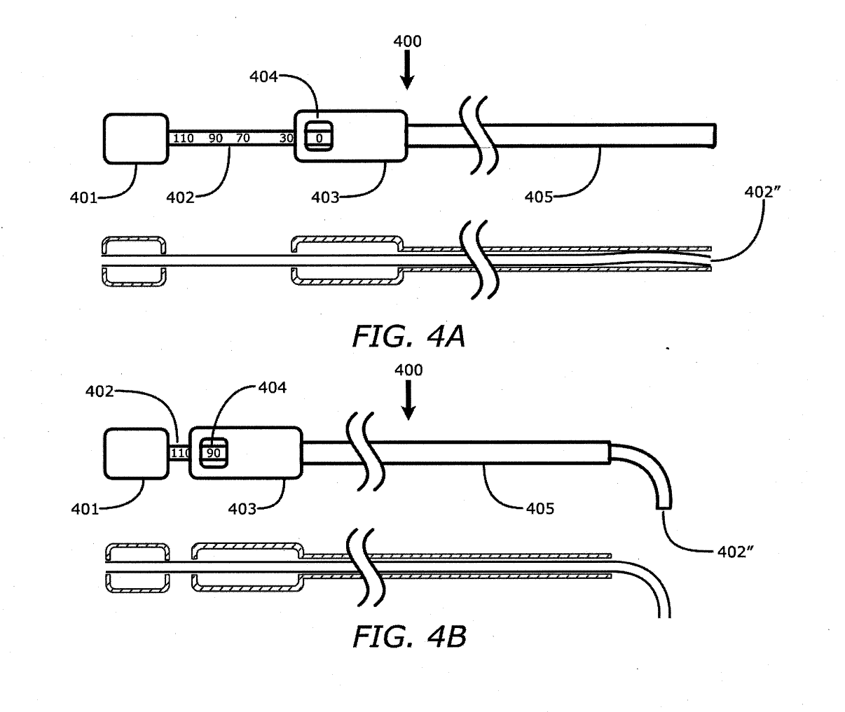

FIGS. 4-11 depict aspects of the invention that includes tip indicator or indication mechanisms that allow an operator to discern the shape and angle or direction of the tip of the transport member without direct visualization of the end of the transport member. For example, these aspects of the invention can provide a positive confirmation of the shape and orientation of the distal end of the transport member when it is desirable to limit the exposure of the patient to x-rays, to expedite procedure times or when direct or indirect visualization is impractical, impossible or not desired. While these embodiments are described using the steerable guide 100 of FIGS. 1A-1C as an example, they can be paired with any or all of the steerable guide embodiments of this invention.

FIGS. 4A-4B depicts markings or indications that could be molded, printed, inscribed, or the like on the transport member body in this embodiment. These markings or reference inscriptions could provide the physician with an indicator of the approximate angle of the distal tip of the transport member 402″ with respect to the longitudinal axis of the cannula 405 when the cannula hub 403 is aligned with the indicator on the transport member shaft 402. The cannula hub 403 may comprise a window 404 that allows viewing of the indicators/markers on the transport member 402 through the cannula hub 403. As shown in FIG. 4B, lining up window 404 in cannula hub 403 with a line or indicator on the transport member 402 that reads ninety (90) degrees could yield an outcome where the cannula 405 is positioned such that an adequate amount of preformed shape of the transport member 402 is unconstrained to provide an approximately 90 degree tip angle of the transport member tip 402″. Alternatively, in the absence of window 404, the proximal edge or end of transport member hub 403 can be simply lined up with the indicator or marker on transport member 402 to achieve a similar outcome. The physician could infer from this marking that the tip angle is set at 90 degrees and that any working tools place into the lumen of transport member 401 could traverse the transport member lumen's straight segment and exit its distal tip 402″ at approximately 90 degrees with respect to the longitudinal axis of cannula 405.

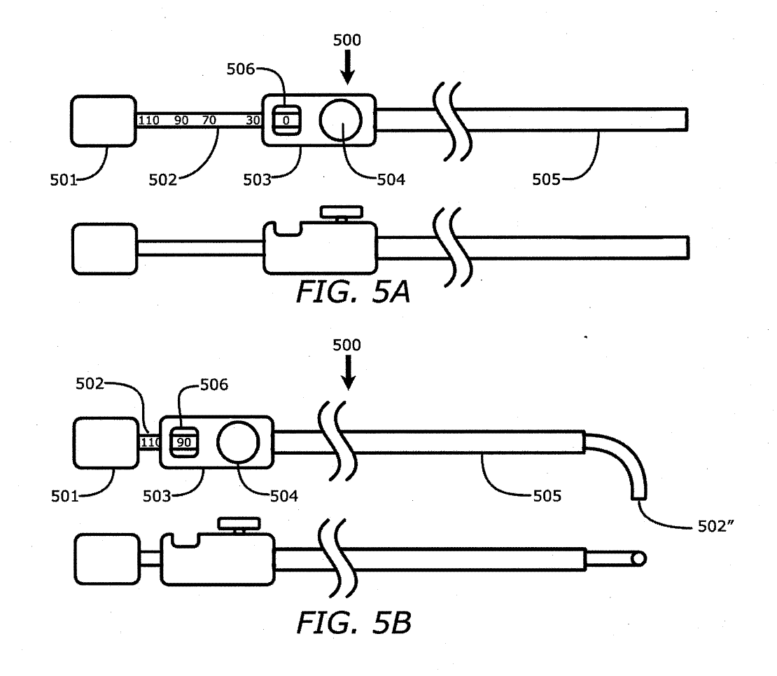

FIGS. 5A-5B depicts the addition of a set screw 504 to the cannula hub 503 in addition to the viewing window 506. By tightening set screw 504, the physician can lock the position of transport member with indicators and hub 501, 502 with respect to cannula 505. As an example, FIG. 5B shows the steerable guide 500 locked in a configuration where the viewing window 506 is aligned with the 90 (ninety) degree marking, thus visually indicating to the physician that the distal tip of the transport member 502″ is positioned approximately perpendicular to the longitudinal axis of cannula 505.

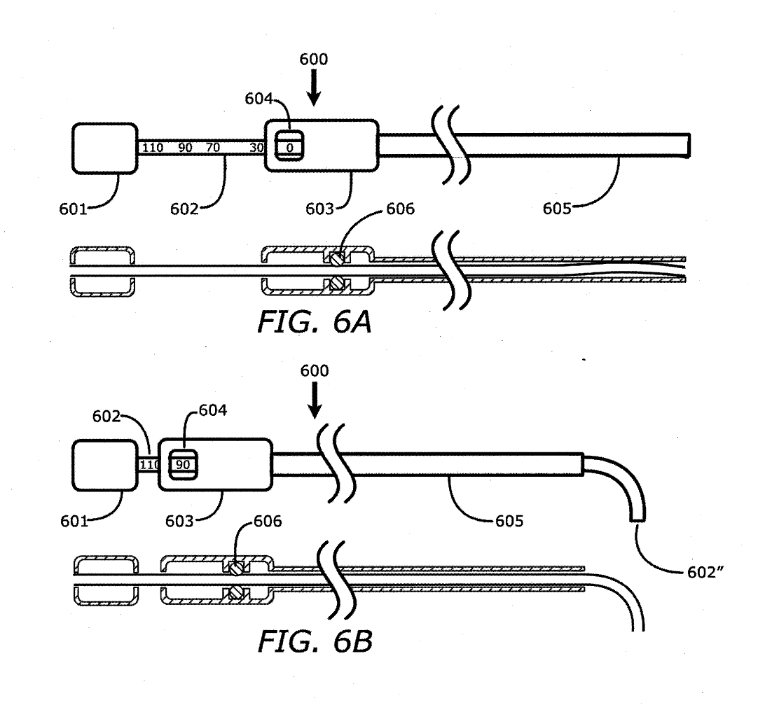

FIGS. 6A-6B depicts an alternative embodiment of the tip indicator mechanism 600 in which a frictional member 606 (e.g. an o-ring) is held in a groove within the cannula hub 603. The frictional member 606 provides a connecting element between cannula hub 603 and transport member & hub 601, 602. The degree of friction or interference between frictional member 603 and transport member 602 dictates the force required to slide the cannula 605 over 602. In this embodiment, the angle of the transport tip distal tip 602″ is read by looking at the indications on transport member 602 through window 604 in cannula hub 603. Alternatively, the edge of the cannula hub 603 could be aligned with the edge of the desired indication on transport member 602.

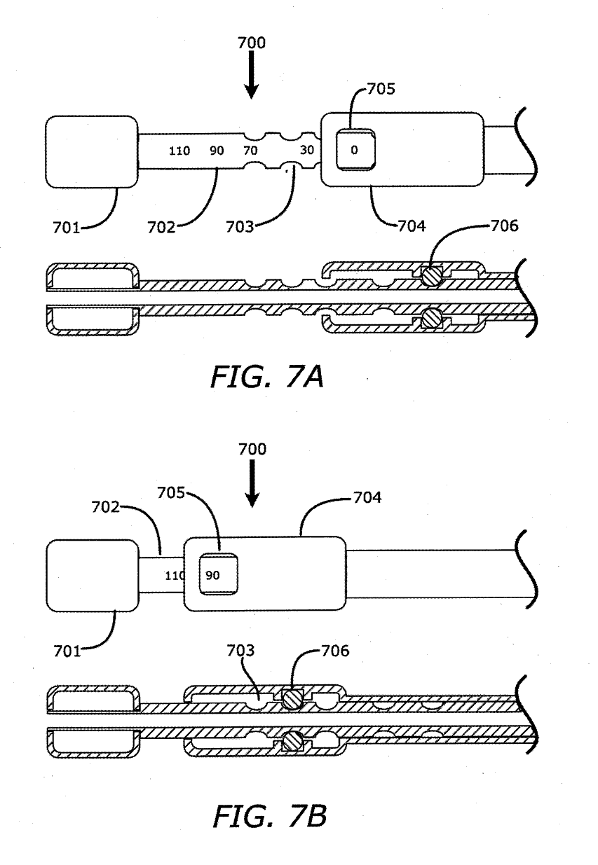

FIGS. 7A-7B depict an embodiment of the invention in which the transport member 702 has detents 703 disposed along the length of the transport member 702 that correspond to the tip angle markers or indications on transport member 702. The detents 703 comprise a path that traverses the circumference of the surface of the transport member 702. This allows the transport member 702 to freely rotate 360 degrees clockwise or counter-clockwise within the cannula member (not shown). The detents 703 engage the frictional member 706 held in cannula hub 704 to provide an additional tactile indication of the configuration of the distal end of the transport member. For example, FIG. 7A shows a configuration of steerable guide 700 in which the cannula hub 704 is aligned such that window 705 allows sight of the visual indicator depicting a 0 (zero) degree transport member & hub 701, 702 distal tip angle (not shown). The frictional member 706 is resting in the distal-most detent 703 corresponding to the transport member tip angle (“0”) marked on transport member 702 and displayed in window 705. The retraction of cannula hub 704 to the position shown in FIG. 7B would be indicated by two signals; one would be the appearance of the visual indicator for a 90 (ninety) degree tip angle marked on transport member 702 and displayed in window 705, the other signal would be a tactile sensation of the frictional member 706 riding over the two detents proximal to the starting detent and settling in the detent corresponding to the 90 (degree) tip angle visual indicator.

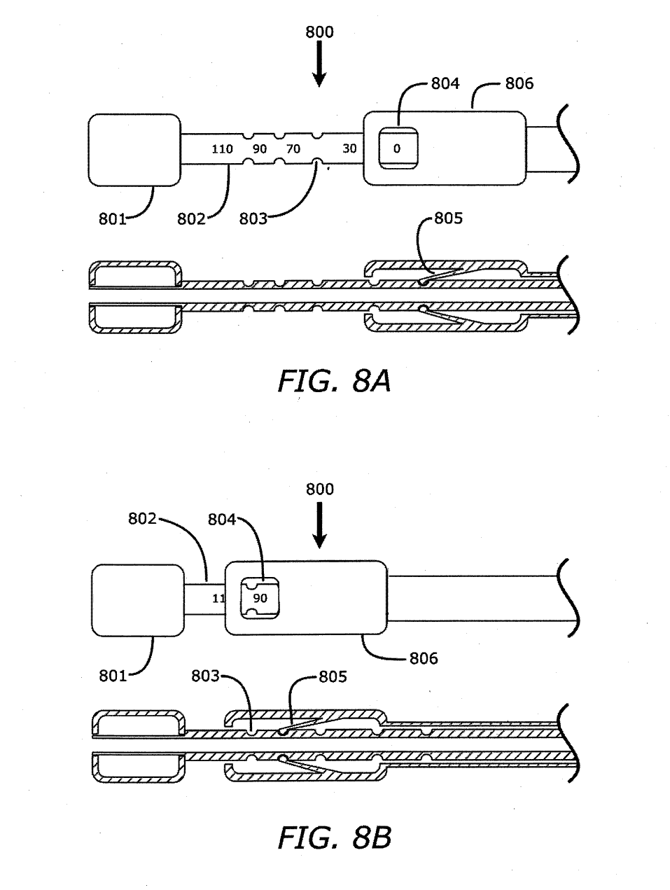

In FIGS. 8A-8B, steerable guide 800 depicts an embodiment where the frictional member 706 depicted in FIGS. 7A-7B is replaced with a ratchet-type mechanism 805. The ratchet mechanism 805 can engage with the detents 803 disposed along the length of transport member & hub 801, 802 to provide tactile feedback conveying information on the state of the distal tip of the transport member in addition to the visual indication provided by the view of the angle marker in window 804. The ratchet means 805 could consist of a living hinge of molded plastic or formed metal designed to deflect and recoil into the detents 803 when the cannula hub 806 is appropriately advanced or retracted. Also, the engagement of the ratchet mechanism 805 into detents 803 could provide an audible signal like a “click” to provide additional feedback to the physician above and beyond the visual and tactile signals mentioned previously.

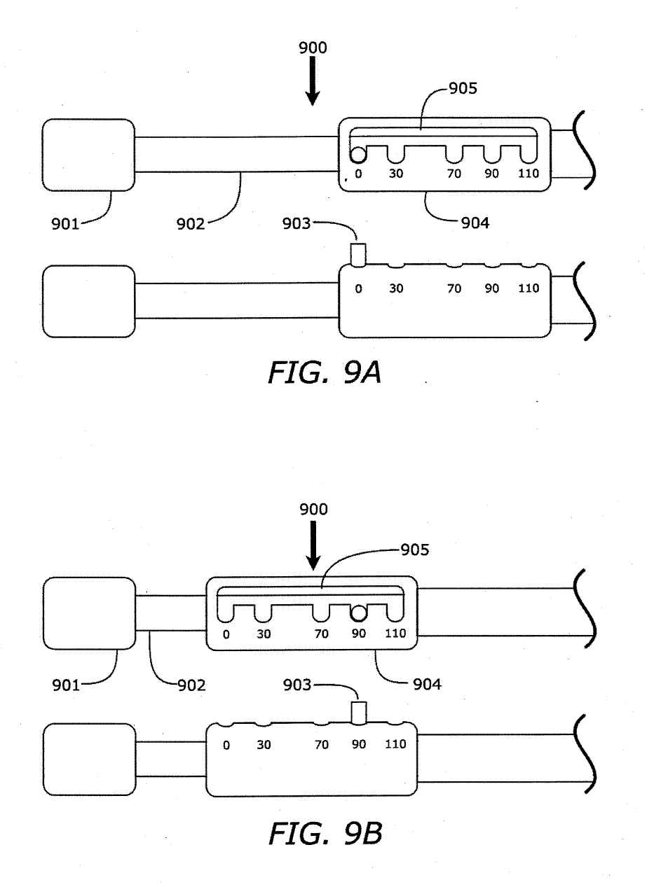

Yet another embodiment of the invention is depicted in FIGS. 9A-9B. The steerable guide system 900 comprises a transport member 902, transport member hub 901, cannula (not shown), and cannula hub 904 in the general form as depicted for steerable guide 100. The transport member 902 has a key 903 affixed to (or extruded from) the body of the transport member 902 that can engage the keyway 905 cut out of cannula hub 904. The keyway 905 is arranged such that the key 903 can be fixed into individual slots of the keyway 905 that represent different transport member distal tip shapes or geometries. In the example shown in FIG. 9A, the key 903 is positioned in the most-proximal slot of keyway 905. By maintaining the key 903 in this position, labeled zero (“0”), the operator is given the information that the transport member 902 is flush with the cannula, and that the distal tip of the transport member 902 has taken the shape of the cannula. In this example, the cannula has a straight configuration resulting in an approximately 0 (zero) degree angle between the distal tip of the transport member 902 and the longitudinal axis of the cannula. This angle can be altered by rotating the transport member 902 by grasping the transport member hub 901 and rotating the transport member hub 901 counterclockwise while holding the cannula hub 904 fixed. This moves key 903 out of the zero (0) degree slot and allows slidable translation of the transport member 902 with respect to the cannula (not shown) to a new or desired position or indication on cannula hub 904. The transport member hub 901 can then be advanced distally and the key 903 re-positioned in one of the more-distal slots by rotating the transport member hub 901 clockwise to fix the position of the transport member 902 with respect to the cannula hub 904. FIG. 9B is an example in which the key 903 has been positioned in the ninety (90) degree slot, indicating that there is an approximately 90 (ninety) degree angle between the distal tip of the transport member 902 and the longitudinal axis of the cannula. Alternatively, the same result can be obtained by holding the transport member 902 in a fixed position, rotating cannula hub 904 clockwise to free the key 903 from the zero (0) degree slot, retracting the cannula hub 904 proximally until the key 903 is aligned with the ninety (90) degree slot in the keyway 905, and rotating the cannula hub 904 counter-clockwise to obtain the configuration shown in FIG. 9B.

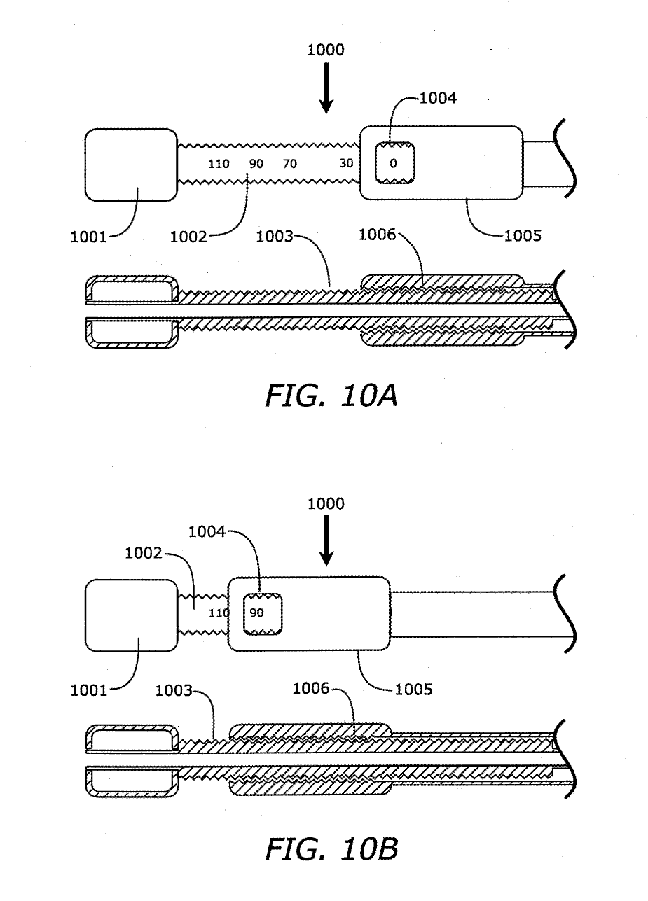

FIGS. 10A-10B depicts an embodiment of the invention in which transport member 1002 has been machined with an angle and pitch 1003 that complements the tapped thread 1006 of the cannula hub 1005. The shape of the distal tip of the transport member & hub 1001, 1002 can be adjusted by rotating the cannula hub 1005 with respect to the transport member 1002. In the example shown in FIGS. 10A-10B, the transport member is 1002 initially flush with the cannula (FIG. 10A) as indicated by the zero (0) degree marker on transport member 1002 and visible in window 1004. The cannula hub 1005 is then rotated relative to the transport member 1002 to retract the cannula in the proximal direction with respect to the transport member 1002 and expose progressively more of the distal section of the transport member 1002. In the final position shown in FIG. 10B, the cannula has been retracted until the window 1004 displays the marker indicating that there is an approximately 90 (ninety) degree angle between the distal tip of the transport member 1002 and the longitudinal axis of the cannula. The angle could then be reverted toward zero degrees (shown as “0” on transport member 1002) by rotating the cannula hub 1005 in the opposite direction.

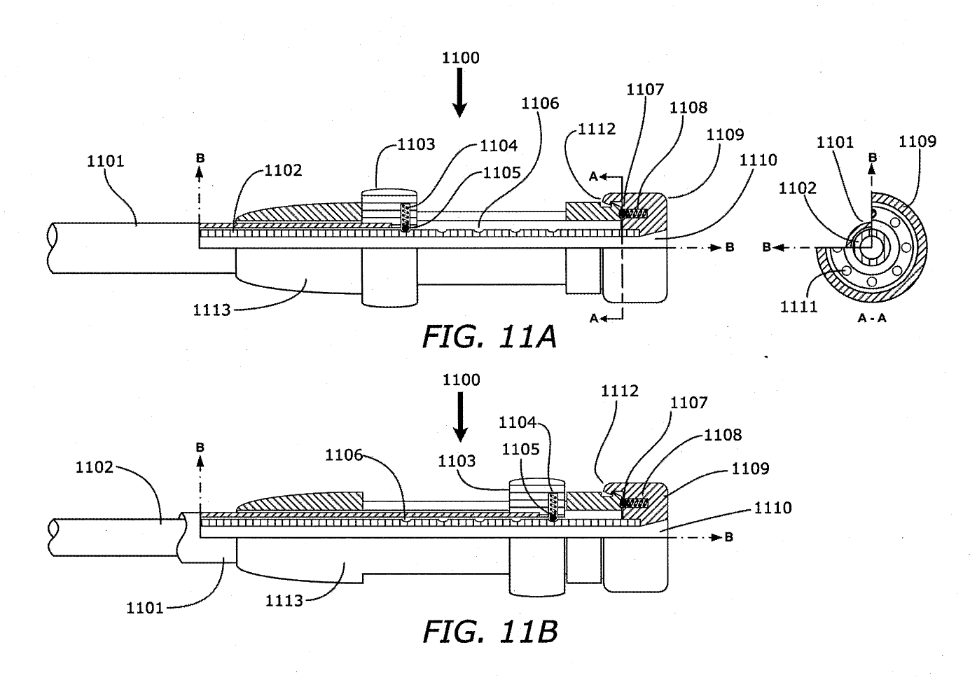

FIGS. 11A and 11B depict another embodiment of the invention showing the tip control mechanism 1100 as an adapter assembly connected at the proximal end of the cannula 1101 and transport member 1102, the configuration of which is useable to the design shown in FIGS. 1A-1C. Alternatively, the general design of the control adapter assembly 1100 can be utilized when the cannula 1101 and the transport member 1102 are switched around, as shown in the design under FIGS. 2A-2C or FIGS. 3A-3C. Referring to FIG. 11A, the control adapter assembly 1100 is comprised of a sliding knob 1103, which contains a spring 1104 and a track ball 1105 mounted in a channel inside the sliding knob 1103. The spring 1104 presses down the track ball 1105 such that when the track ball 1105 is aligned and engages with one of detent groves 1106, the sliding knob 1103 will be in a fixed position with respect to movement in longitudinal axis direction, providing tactile and/or audible feedback to the user. The sliding knob 1103 is attached to the cannula 1101, providing direct control to the longitudinal movement of the cannula 1101, such that when the sliding knob 1103 is retracted in the proximal direction as shown in FIG. 11B, the cannula 1101 moves in the same direction and distance. Retracting the sliding knob 1103 simultaneously exposes the transport member distal end (not shown in these figures) to assume a pre-configured shape. Each detent groove 1106 disposed along the outside surface of the transport member proximal segment may signify a tip curve or shape, the most distal detent groove 1106 represents the maximum tip angle or shape and the most proximal detent groove 1106 represents the tip angle or shape in a relatively straight configuration. Each detent groove 1106 positioned in between the most distal and most proximal positions represent a pre-determined tip angle or shape at the distal end of the transport member 1102. A label or markings or indications (not shown) that could be molded, printed, inscribed, or the like that provides visual indication to the user may be added in the control adapter 1100 as primary or secondary tip angle or shape indicator. The detent groove 1106 may partially or fully cover the circumference of the transport member's 1102 proximal end to allow radial motion or rotation of the transport member. The rotational motion of the transport member 1102 is controlled by the rotating cap 1109 where the proximal end of the transport member 1102 is attached. As shown in FIG. 11A, the rotating cap 1109, secured at the proximal end of control adapter body 1113 by means of a snap fit 1112, contains a spring 1108 and a track ball 1107 mounted in a channel of the rotating cap 1109. The spring 1108 presses down the track ball 1107 such that when the track ball 1107 is aligned and engages with one of detent grooves 1111 (FIG. 11A, section A-A), the rotating cap 1109 will be in a fixed position with respect to movement in rotational direction, providing a tactile and/or audible feedback to the user. Each detent groove 1111 positioned around the proximal end of control adapter body 1113 (FIG. 11A, section A-A) is disposed to indicate the relative direction of the transport member tip with respect to a zero degrees position reference (not shown). Alternatively a label or markings or indications (not shown) that could be molded, printed, inscribed, or the like that provides visual indication to the user may be added in the control adapter 1100 as primary or secondary tip position (or direction) indicator. At the proximal end of the rotating cap 1109, a lumen funnel opening 1110 is provided to allow ease of introduction of devices being inserted through the transport member. Alternatively, a luer port adapter (not shown) may be attached or provided or integrated with the lumen funnel opening 1110 to allow attachment of other accessories or devices at the proximal end of the control adapter 1100. Any of the embodiments relating to the control means of indicating the tip shape or direction as described in this invention can be applied to the control adapter of tip indicator mechanism 1100. The embodiment of tip indicator mechanism 1100 may be configured to allow single handed adjustment of the sliding knob 1103 and the rotating cap 1109. There are numerous ergonomic options that could be employed for the design to achieve the single handed adjustment capability and FIG. 11A and FIG. 11B serve as exemplary embodiments.

Yet another embodiment of the tip indicator mechanism of the invention (not shown) may employ a rack and pinion system to control the angle of the tip of the transport member. The pinion may be mounted in the hub of the cannula, with the gear teeth of the pinion engaging the gear teeth of the rack mounted over the outer surface of the transport member. The shaft of the pinion may extend through the wall of the hub and terminate in a control knob or wheel or similar means of activation. Rotation of the control knob or wheel will rotate the teeth of the pinion to advance or retract the rack and transport member with respect to the cannula. The control knob or wheel may have reference markings or indicators inscribed or otherwise affixed to the surface or edge of the control knob or wheel that relay information to the user about the tip angle of the transport member with respect to the longitudinal axis of the transport member. For example, the knob may have markings that indicate tip angles of 0 degrees, 30 degrees, 70 degrees, 90 degrees and 110 degrees. These markings may be referenced against a line, dot, or other indicator inscribed or otherwise applied to the hub of the cannula. In another example, the control knob or wheel may have a reference line, dot, or other indicator inscribed or otherwise applied to or on the surface or edge of the control knob or wheel. For example, the hub of the cannula may have markings that indicate tip angles of 0 degrees, 30 degrees, 70 degrees, 90 degrees and 110 degrees. Alignment of the reference mark on the control knob or wheel with the desired tip angle marking would produce the corresponding angle between the transport member tip and the longitudinal axis of the transport member.

The rack component of this embodiment of the invention may have a geometry that is suitable to the desired level of control over tip alignment in the steerable guide. For example, an embodiment of the steerable guide that is intended to control translation of the transport member with respect to the cannula (and thus the angle between the tip and longitudinal axis of the transport member) may use a rack with a square cross section. In another example, an embodiment of the steerable guide that is intended to control both translation of the transport member with respect to the cannula and radial rotation of the transport member with respect to the cannula may use a circular rack with gear teeth provided around the outer circumference of perimeter of the circular rack. In this example, the transport member is mounted through a channel or lumen axially disposed along the center of the rack. A circular rack allows the transport member to rotate within the cannula while maintaining engagement between the rack and pinion.

While the preceding description uses a rack and pinion structure as an illustration of the concept of transforming rotational motion of a control member into translation of the transport member with respect to the cannula, any gear mechanism may be employed to achieve this end. For example, the rack and pinion may be replaced by a tongue and groove mechanism or a rotating pin and groove mechanism. Bevel gears may be used to change the physical location and/or orientation of the control knob with respect to the cannula hub and/or the transport member shaft. Additional gears may be incorporated into the design to change the gear ratio between the control knob and the rack. Furthermore, though the preceding description of this embodiment was framed using a steerable guide system 100 as described in FIGS. 1A-1C, these designs are equally applicable to a steerable guide system 200 as described in FIGS. 2A-2C. In the case of steerable guide system 200, the control knob or wheel may be mounted on the transport member hub, the rack may be on the outer surface of the cannula, and rotation of the control knob or wheel will retract or advance the cannula with respect to the transport member. The incorporation of detents, living hinges, spring and ball systems, rotational control mechanisms and other aspects described above are equally applicable to steerable guide 200.

A further embodiment of the tip indicator mechanism (not shown) comprises a winch system to control the angle of the tip of the transport member. The winch may be anchored to the substantially rigid cannula, with the one end of the cable fixed to the spool and the other end of the cable fixed to the proximal portion of the transport member. Rotation of the spool will either wind the cable and advance the transport member distally with respect to the cannula, or unwind the cable and retract the transport member proximally with respect to the cannula. The winch may be surrounded by a housing or handle. A control knob or wheel may be located on the exterior of the housing or handle, with an axle running through a space or hole in the housing or handle and fixed to the winch spool. Rotation of the control knob or wheel will rotate the winch spool to affect advancement or retraction of the transfer tube with respect to the substantially rigid cannula. A series of gears may be positioned between the control knob or wheel and the winch spool to increase spool torque and decrease winding or unwinding speed or decrease spool torque and increase winding or unwinding speed. The cable may be comprised of a material that can withstand the tensile and compressive loads applied by the winch including, but not limited to nitinol, stainless steel, polymer or plastic (e.g. nylon), composites, and the like. The form of the cable may include, but is not limited to a single wire, braided wire, flat wire, coiled wire, and the like. The cable may be fixed to the transport member via methods known in the art including, but not limited to bonding, crimping, swaging, press fit, screw or bolt and the like. Alternatively, the end of the cable attached to the transport member may float in a groove, ring, and/or channel to enable the transport member to rotate axially with respect to the substantially rigid cannula while supporting translational motion.

Though the preceding description of this embodiment was framed using a steerable guide system 100 as described in FIGS. 1A-1C, these designs are equally applicable to a steerable guide system 200 as described in FIGS. 2A-2C. In the case of steerable guide system 200, the winch may be mounted on the transport member hub with the one end of the cable fixed to the spool and the other end of the cable fixed to the proximal portion of the substantially rigid cannula. Rotation of the spool will either wind up the cable and advance the cannula distally with respect to the transport member, or wind out the cable and retract the cannula proximally with respect to the transport member. The winch may be surrounded by a housing or handle. A control knob or wheel may be located on the exterior of the housing or handle, with an axle running through a space or hole in the housing or handle and fixed to the winch spool and rotation of the control knob or wheel will retract or advance the cannula with respect to the transport member. The incorporation of detents, living hinges, spring and ball systems, rotational control mechanisms and other aspects described above are equally applicable to steerable guide 200.

The control knob or wheel in any of the rack and pinion or winch systems previously described may have reference markings or indicators inscribed or otherwise affixed to the surface or edge of the control knob or wheel that relay information to the use about the angle of the transport member tip with respect to the longitudinal axis of the transport member. For example, the knob may have markings that indicate tip angles of 0 degrees, 30 degrees, 70 degrees, 90 degrees and 110 degrees. These markings may be referenced against a line, dot, or other indicator inscribed or otherwise applied to the hub, handle, or housing. In another example, the control knob or wheel may have a reference line, dot, or other indicator inscribed or otherwise applied to or on the surface or edge of the control knob or wheel. The hub, handle or housing may have markings that indicate tip angles of 0 degrees, 30 degrees, 70 degrees, 90 degrees and 110 degrees. Alignment of the reference mark on the control knob or wheel with the desired tip angle marking would produce the corresponding angle between the tip and the longitudinal axis of the transport member.

Alternatively, the control knob or wheel may have a series of detents spaced around the control knob or wheel that correspond to the markings that indicate the tip angles of the transport member with respect to the longitudinal axis of the transport member. The hub, handle, or housing may have at least one living hinge (i.e. an elastically deformable hinge) such as the ratchet mechanism 805 shown in FIGS. 8A and 8B for example, that engages each detent as the control knob or wheel is rotated clockwise or counter-clockwise as desired to provide tactile and/or audible feedback to the user. In another embodiment, the cannula hub may contain at least one spring and at least one track ball, such as the spring 1104 and the track ball 1105 shown in FIGS. 11A and 11B for example, mounted in a channel of the hub, handle, or housing. The spring presses the track ball against the control knob or wheel such that when the ball is aligned with and engages one of the detents, the control knob or wheel will be in a fixed position with respect to rotation (and thus the transport member will be fixed with respect to translation), providing tactile and/or audible feedback to the user. In both of these examples, the location of the detent and engaging mechanism (living hinge or ball and spring) may be reversed. For example, the detents may be located on the hub, handle, or housing and the living hinge may be located on the control knob or wheel.

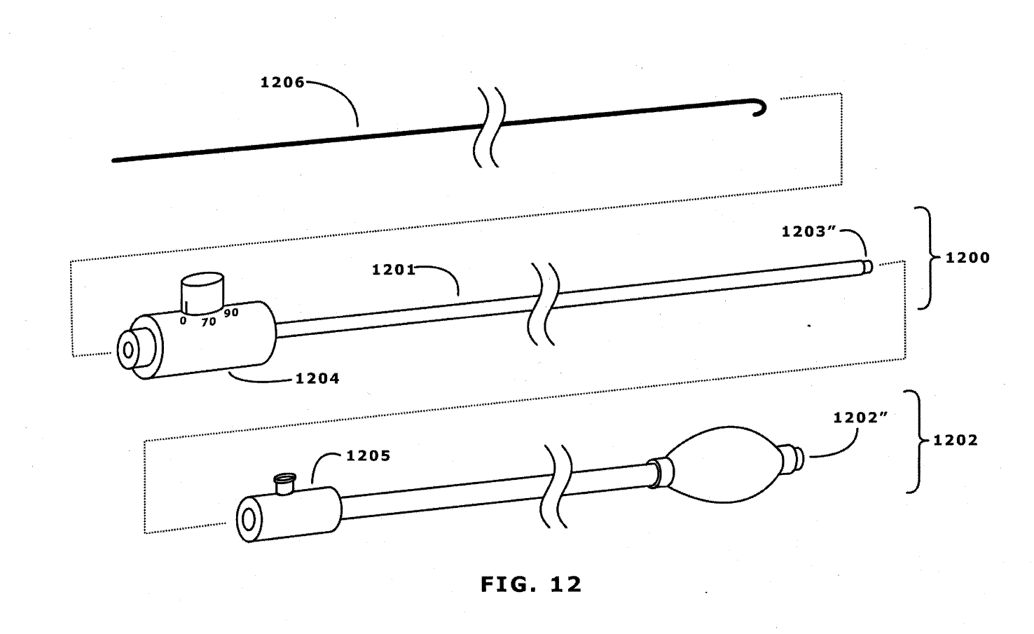

FIG. 12 depicts another embodiment of the steerable elongate guide system 1200 wherein the outer diameter of the cannula 1201 is sized to fit within the lumen of an over the wire balloon catheter 1202. The over the wire balloon catheter 1202 may be of the design disclosed in co-pending U.S. Pat. App. No. 61/352,244 herein incorporated in full by reference. The length of the cannula 1201 and the transport member may be longer than the overall length of the balloon catheter 1202 such that the distal tip of the transport member 1203″ extends beyond the distal tip of the balloon catheter 1202″. The steerable guide system cannula hub 1204 may be configured to reversibly connect with the balloon catheter hub 1205 such that the steerable guide system 1200 may be inserted into the over the wire balloon catheter 1202 and reversibly lock the cannula hub 1204 to the balloon catheter hub 1205, thus enabling an operator to use the combined devices as a single unit. The steerable elongate guide system cannula hub 1204 also features a rheostat-like tip indicator mechanism showing the tip deflection angle at the distal end (shown at 0 degrees in FIG. 12). Clockwise or counterclockwise rotation of the rheostat-like switch or tip indicator mechanism relative to the hub body to the marked angle (e.g. 0 degrees, 30 degrees, 70 degrees, 90 degrees shown in FIG. 12) produces approximately the same tip deflection at the transport member's distal tip 1203″. The releasable connection may be achieved through the use of mechanisms that include, but are not limited to living hinges, magnets, detents, spring and levers, spring and balls, rotating collars or collets, key and keyhole mechanisms, screws and taps, compliant or semicompliant rings or gaskets, and the like. A guidewire 1206 may be inserted into the lumen of the transport member 1200 to enable placement of the guidewire into the target anatomy, such as into or through a sinus ostium.

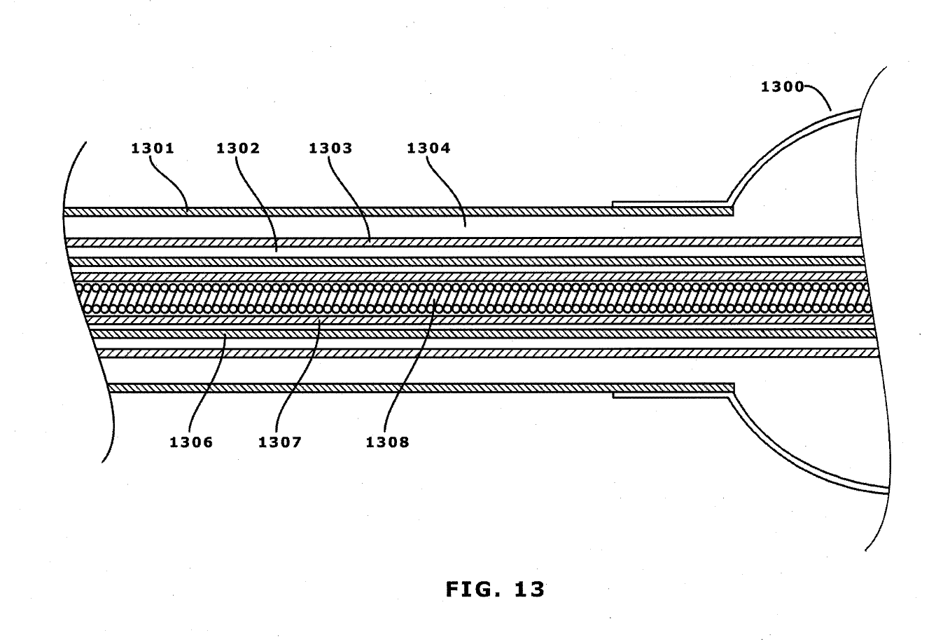

FIG. 13 depicts a detailed cross-sectional view of the steerable elongate guide system 1200 inserted into the guidewire lumen of an over-the-wire balloon catheter 1202 along with a guidewire 1308 position within the lumen of transport member 1307 of the steerable elongate guide system 1200. The balloon catheter 1202 in this figure comprises an expandable balloon segment 1300, a catheter shaft 1301, and an inner lumen 1302 defined by an internal elongate member 1303. The expandable balloon segment 1300 is in fluid communication with the luminal space 1304 between the catheter shaft 1301 and the internal elongate member 1303. After insertion into balloon catheter 1202, the steerable guide system 1200 resides in the inner lumen 1302. The cannula 1306 is sized to be slidably disposed within lumen 1302. As described previously, transport member 1307 is slidably disposed within cannula 1306. The relative linear and rotational motion of cannula 1306 with respect to transport member 1307 serves to adjust the angle of the transport member tip (not shown) with the longitudinal axis of the transport member 1307 and the rotational orientation of the transport member 1307 with respect to the cannula 1306. In this example, transport member 1307 comprises a lumen that may be sized to accept an appropriate guidewire 1308 or other mandrel.

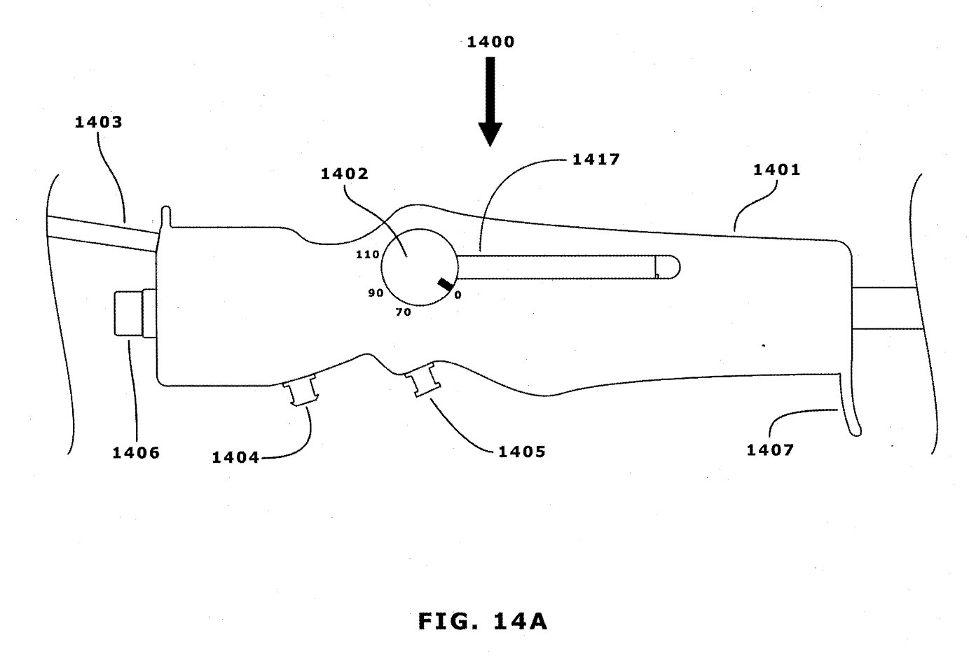

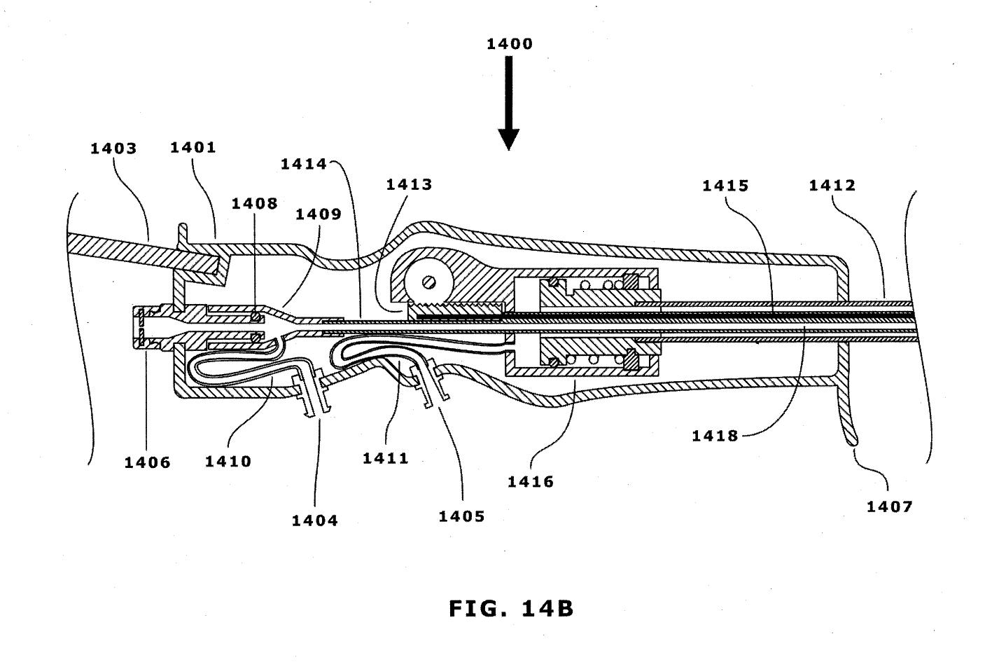

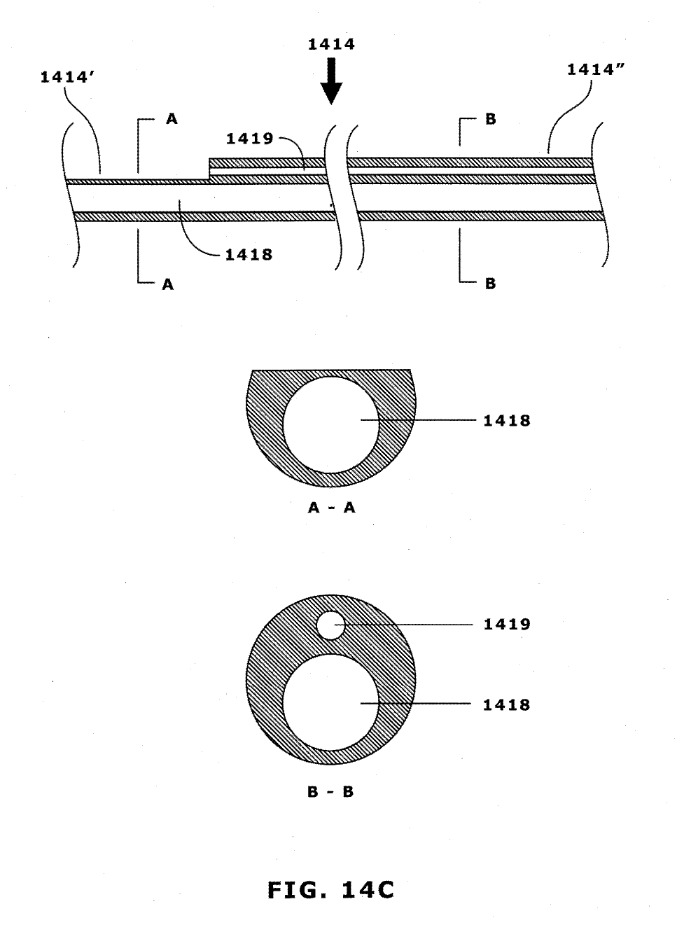

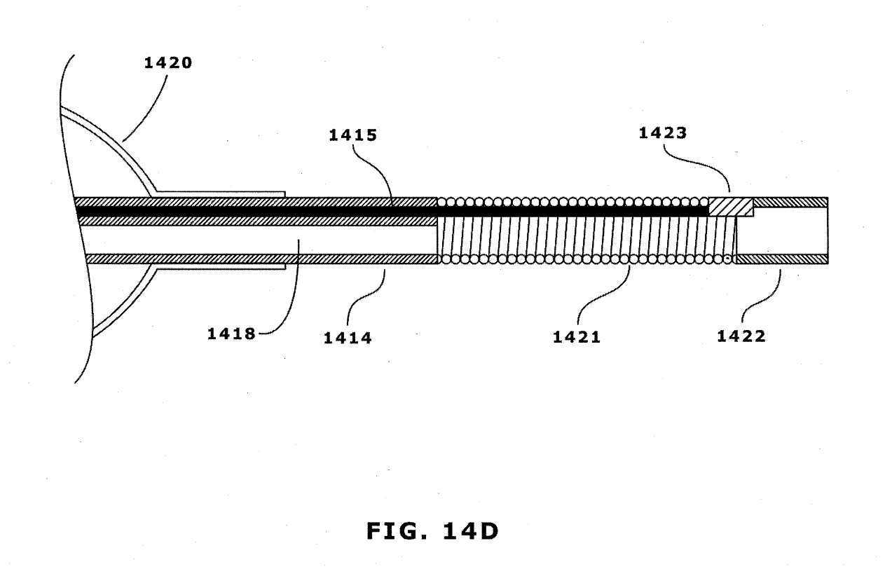

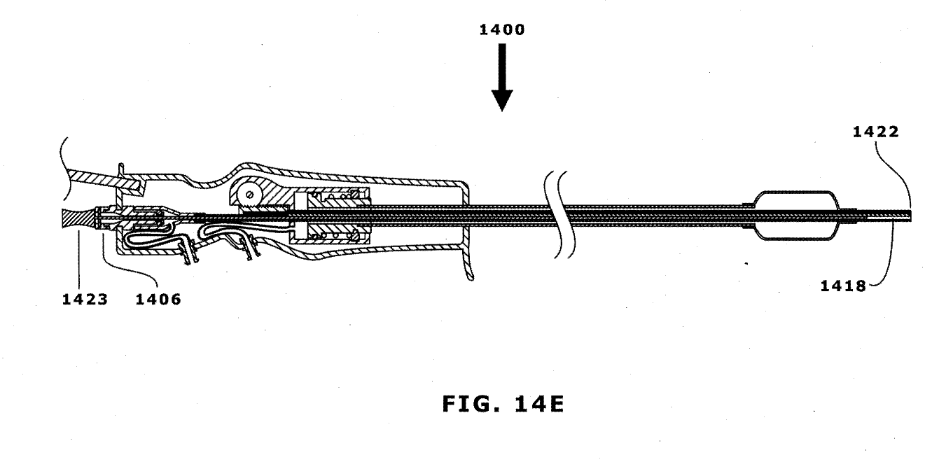

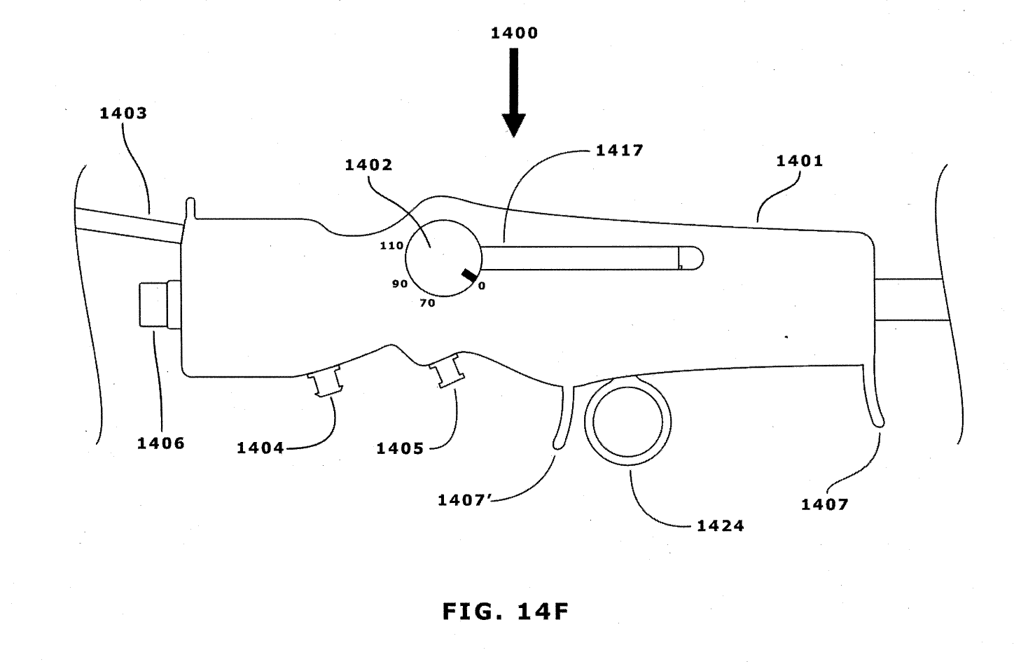

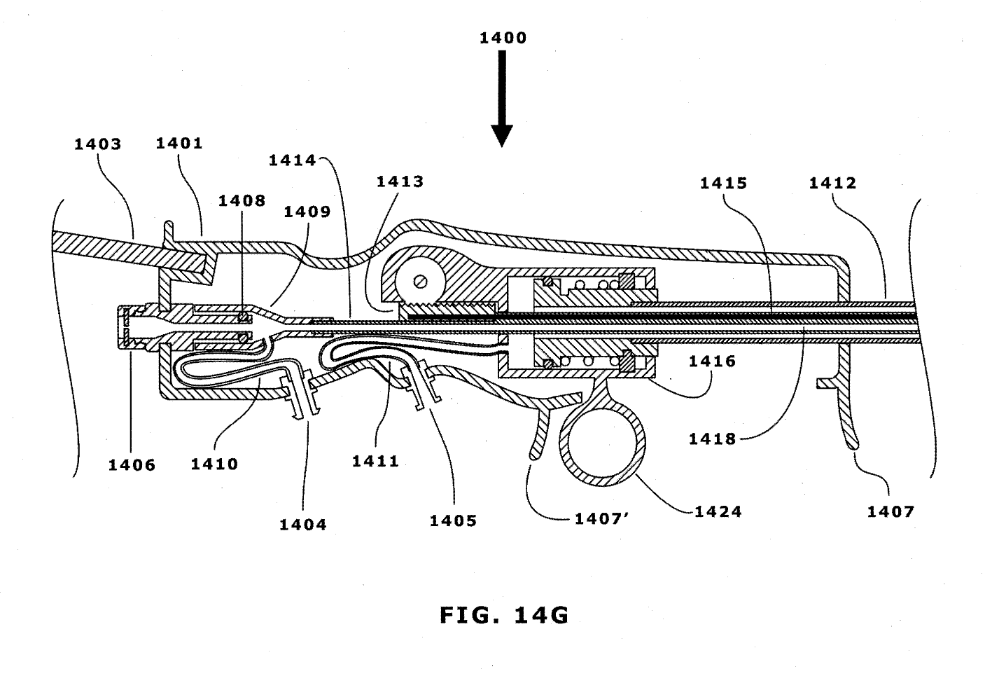

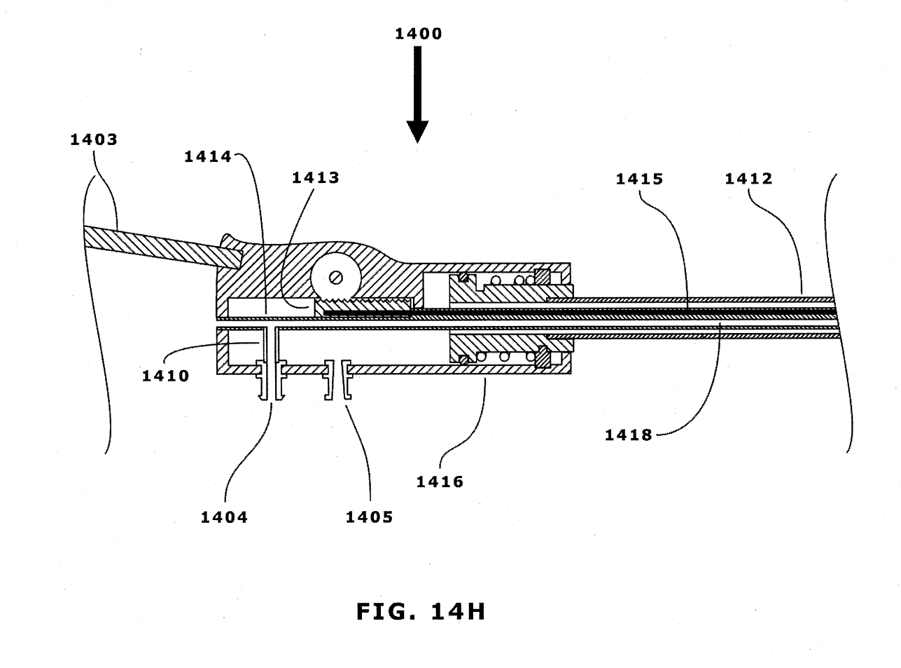

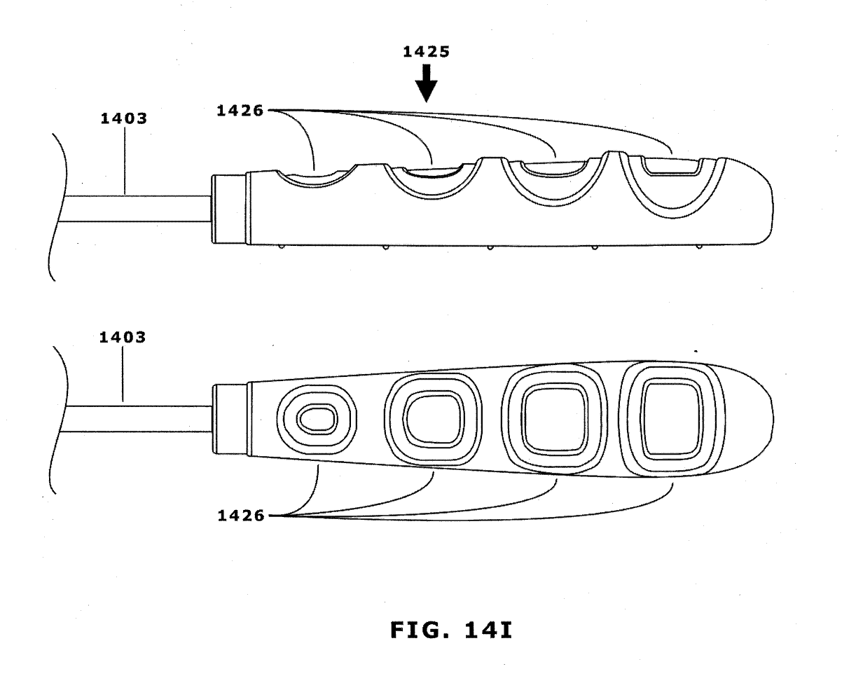

FIGS. 14A-14D depict side, cross-sectional, and sectioned views of an embodiment of a steerable balloon catheter of the invention. The steerable balloon catheter 1400 comprises a shell 1401, a flexible handle extension 1403, a control knob or adaptor 1402, a guidewire retaining valve 1406, an aspiration port 1404, and an inflation port 1405 as shown in FIG. 14A. The shell 1401 may be fabricated using methods known in the art including, but not limited to machining, molding, stereolithography, and the like from materials known in the art including PMMA, polycarbonate, Pebax, nylon, ABS, stainless steel, aluminum, anodized aluminum, titanium, and the like. The shell 1401 further comprises a flange 1407 and a window 1417. In this embodiment, the flange 1407 serves as an anchor point to enable a one-handed action to slide the control knob 1402 along window 1417 in the distal or proximal directions. While depicted as a flange, feature 1407 may alternatively comprise at least one ring, grip, indentation, wing, or other structure that may be used with rotating and/or translating knob 1402 to ease advancement or retraction of control knob 1402 along window 1417. Window 1417 may be fabricated using methods known in the art including, but not limited to machining, molding, electrical deposition machining, and the like.

FIG. 14B depicts the internal components within the shell 1401 of steerable balloon catheter 1400. Balloon control hub 1416 comprises the components required for the inflation and deflation of a balloon catheter. As shown in this example, the components within balloon control hub 1416 are those denoted for a regrooming balloon catheter as disclosed in co-pending U.S. Pat. App. No. 61/352,244 herein incorporated in full by reference. The distal end of inflation tube 1411 is connected to and in fluid and/or air communication with balloon control hub 1416. Inflation tube 1411 may be an elongate flexible member with at least one lumen fabricated from materials known in the art including, but not limited to nylon, polyurethane, silicone rubber, polyethylene, Viton®, neoprene rubber, EPDM, nitrile, rubber, PTFE, EVA, PVC, PVDF, Tygon, and the like. Alternatively, this tubing could be reinforced using methods known in the art including, but not limited to braiding, coils, laminates, and the like. The proximal end of inflation tube 1411 is joined to inflation port 1405 using methods known in the art including, but not limited to adhesive bonding, ultrasonic welding, overmolding, and the like. Inflation port 1405 may consist of one of any standard connector including, but not limited to luer locks, hose barbs, threaded fittings, etc. and may be fabricated from materials known in the art including, but not limited to nylon, polyurethane, acrylic, polycarbonate, polyimide, PET, PEEK, polyolefin, PTFE, Pebax, Delrin, polyethylene, stainless steel, nitinol, and combinations thereof.

The balloon shaft 1412 and multi-lumen tubing 1414 are arranged coaxially; the lumen between balloon shaft 1412 and multi-lumen tubing 1414 acts as the inflation and/or deflation lumen of balloon 1420 (shown in FIG. 14D). Balloon shaft 1412 may be comprised of materials known in the art including, but not limited to nylon, polyurethane, polycarbonate, polyimide, PET, PEEK, polyolefin, PTFE, Pebax, Delrin, polyethylene, stainless steel, nitinol, and combinations thereof. Balloon shaft 1412 may be reinforced by methods known in the art including, but not limited to a braid, coil, or the like, or may have a surface coating to modify its lubricity. The outer surface of multi-lumen tubing 1414 acts as the inner wall of the balloon inflation and deflation lumen. In this example, multi-lumen tubing 1414 comprises two lumens; one contains pull wire 1415, the other acts as an aspiration or guidewire lumen 1418. While multi-lumen tubing 1414 is shown as comprising two lumens in FIG. 14B, it should be obvious to those of skill in the art that multi-lumen tubing 1414 may possess any number of lumens. The proximal end of multi-lumen tubing 1414 is bonded to sliding hub 1409. The two components may be fixed to each other using techniques known in the art including, but not limited to adhesive bonding, ultrasonic welding, interference fitting, threading, set screw, press fitting, overmolding, crimping, and the like. Multi-lumen tubing 1414 may have a single cross-sectional geometry, stiffness, lubricity, radio-opacity, over its length, or optionally, any or all of the material characteristic of multi-lumen tubing 1414 may vary along its length. For example, the proximal section of multi-lumen tubing 1414 may be relatively stiff, while the distal section of multi-lumen tubing 1414 may be relatively ductile. Alternatively, the geometry of the proximal section of multi-lumen tubing 1414 may be larger in outer diameter while the distal section of multi-lumen tubing 1414 may be smaller in outer diameter. The transition between the different states of each variable characteristic may be abrupt or the transition may be gradual.

A detailed view of the multi-lumen tubing 1414 as embodied in this example is given in FIG. 14C. The proximal portion of multi-lumen tubing 1414′ comprises a single guidewire lumen 1418, as shown in section A-A. The remainder of multi-lumen tubing 1414″ comprises a pullwire lumen 1419 and a guidewire lumen 1418 as shown in section B-B. Multi-lumen tubing 1414 may be fabricated from materials known in the art including, but not limited to nylon, polyurethane, polycarbonate, polyimide, PET, PEEK, polyolefin, PTFE, Pebax, Delrin, polyethylene, stainless steel, nitinol, and combinations thereof.

As shown in FIG. 14B, pullwire 1415 runs through pullwire lumen 1419 and is joined to rack 1413 at its proximal end. Pullwire 1415 may be joined to rack 1413 by methods known in the art including, but not limited to adhesive bonding, ultrasonic welding, set screws, overmolding, crimping and the like. Rack 1413 may be fabricated from materials known in the art including, but not limited to nylon, polyurethane, polycarbonate, polyimide, PET, PEEK, polyolefin, PTFE, Pebax, Delrin, polyethylene, stainless steel, nitinol, and combinations thereof. Rack 1413 interacts with a pinion (not shown) which may be mounted in the balloon hub 1416 with the gear teeth of the pinion engaging the gear teeth of the rack 1413. The shaft of the pinion may extend through the wall of balloon hub 1416 and terminate in control knob 1402 (shown in FIG. 14A) or a similar means of activation. Rotation of control knob 1402 rotates the teeth of the pinion to advance or retract the rack 1413 and pullwire 1415 with respect to the balloon hub 1416 and multi-lumen tubing 1414. For example, retraction of rack 1413 and pullwire 1415 bends flexible segment 1421 (shown in FIG. 14D) and changes the angle of tip 1422 with respect to the longitudinal axis of multi-lumen tubing 1414. Control knob 1402 (shown in FIG. 14A) may have reference markings or indicators inscribed or otherwise affixed to the surface or edge of the control knob 1402 that relay information to the user about the angle of tip 1422 (shown in FIG. 14D) with respect to the longitudinal axis of the multi-lumen tubing 1414. For example, the control knob 1402 (shown in FIG. 14A) may have a reference line, dot, or other indicator inscribed or otherwise applied to its surface. Corresponding markings that indicate tip angles of 0 degrees, 70 degrees, 90 degrees and 110 degrees, for example, may be inscribed, engraved, pad printed, or otherwise applied to shell 1401. Alignment of the reference mark on the control knob 1402 with the desired tip angle marking would produce the corresponding angle between the tip 1422 (shown in FIG. 14D) and the longitudinal axis of the multi-lumen tubing 1414. In another example (not shown), the control knob 1402 may have reference markings or indicators inscribed or otherwise affixed to the surface or edge of the control knob 1402 that relay information to the user about the angle of tip 1422 with respect to the longitudinal axis of multi-lumen tubing 1414. For example, the control knob 1402 may have markings that indicate tip angles of 0 degrees, 70 degrees, 90 degrees and 110 degrees. These markings may be referenced against a line, dot, or other indicator inscribed or otherwise applied to the shell 1401.

Alternatively (not shown), the control knob 1402 may have a series of detents spaced around the control knob 1402 that correspond to the markings that indicate the angle of tip 1422 with respect to the longitudinal axis of the multi-lumen tubing 1414. The shell 1401 or balloon hub 1416 may have at least one living hinge (i.e. an elastically deformable hinge) such as the ratchet mechanism illustrated previously in FIGS. 8A and 8B for example, that engages each detent as the control knob 1402 is rotated clockwise or counter-clockwise as desired to provide tactile and/or audible feedback to the user. In another embodiment, the balloon hub 1416 or shell 1401 may contain at least one spring and at least one track ball, such as those previously shown in FIGS. 11A and 11B for example, mounted in a channel of the balloon hub 1416 or shell 1401. The spring presses the track ball against the control knob 1402 such that when the ball is aligned with and engages one of the detents, the control knob 1402 will be in a fixed position with respect to rotation (and thus the angle of deflection of tip 1422 will be fixed), providing tactile and/or audible feedback to the user. In both of these examples, the location of the detent and engaging mechanism (living hinge or ball and spring) may be reversed. For example, the detents may be located on the balloon hub 1416 or shell 1401 and the living hinge may be located on the control knob 1402. While this example has framed a rack and pinion mechanism as a method for controlling the angle of deflection of tip 1422, it should be clear to one of skill in the art that any of the control mechanisms discussed in this patent are sufficient to control the angle of deflation of tip 1422.

The distal end of one embodiment of the steerable balloon catheter is shown in FIG. 14D. The distal end of pullwire 1415 is joined to the distal end of flexible member 1421 via bond 1423. Bond 1423 may be realized through techniques known in the art including, but not limited to welding, adhesive bonding, crimping, and the like. Flexible member 1421 may be a coiled wire fabricated from materials including, but not limited to stainless steel, nitinol, nylon, PET, polycarbonate, PEBAX, HDPE, polyurethanes, fluoropolymers, composite materials such as PEBAX tubing with embedded braids of nitinol, stainless steel, copper, and the like. The proximal end of flexible member 1421 is joined to the distal end of multi-lumen tubing 1414 using techniques known in the art including, but not limited to adhesive bonding, ultrasonic welding, interference fitting, threading, press fitting, crimping, and the like. The distal end of flexible member 1421 is joined to the proximal end of tip 1422 using techniques known in the art including, but not limited to adhesive bonding, ultrasonic welding, interference fitting, threading, press fitting, crimping, and the like. Tip 1422 comprises an elongate member with at least one lumen extending from its proximal to distal ends. Tip 1422 may be fabricated from materials known in the art including, but not limited to nylon, polyurethane, polycarbonate, polyimide, PET, PEEK, polyolefin, PTFE, Pebax, Delrin, polyethylene, stainless steel, nitinol, and combinations thereof. The distal end of tip 1422 may be shaped into an atraumatic geometry such as but not limited to a taper, hemisphere, ball, and the like. The physical characteristics and geometry of the tip 1422 may be uniform or variable over its length. Additionally, the steerable balloon catheter 1400 may comprise (not shown) marker bands or beacons that allow for visualization of the device using methods known in the art including, but not limited to magnetic modalities, ultrasound, electromagnetic navigation, infrared navigation, computed tomography, fluoroscopy, and the like.

As shown in FIG. 14B, aspiration seal 1408 provides an air and/or fluid tight seal between the proximal segment of sliding hub 1409 and the distal segment of guidewire retaining valve 1406. Aspiration seal 1408 may be an o-ring, gasket or other component or other component fabricated from materials known in the art including, but not limited to polychloroprene, silicone rubber, nitrile rubber, Viton®, EPDM, butyl rubber, natural rubber, polyethylene, and the like. The proximal segment of sliding hub 1409 may be sized to fit coaxially over the distal segment of guidewire retaining valve 1406 as shown in FIG. 14B, or the proximal segment of sliding hub 1409 may be sized to fit coaxially within the distal segment of guidewire retaining valve 1406. Sliding hub 1409 may be fabricated of materials known in the art including, but not limited to nylon, polyurethane, polycarbonate, polyimide, PET, PEEK, polyolefin, PTFE, Pebax, Delrin, polyethylene, stainless steel, nitinol, and combinations thereof. Sliding hub 1409 has a port connected the proximal end of aspiration tube 1410 via methods known in the art including, but not limited to adhesive bonding, ultrasonic welding, overmolding, and the like. Aspiration tube 1410 is an elongate member with at least one lumen and may be fabricated from materials known in the art including, but not limited to nylon, polyurethane, silicone, polyethylene, Viton®, neoprene rubber, EPDM, nitrile, rubber, PTFE, EVA, PVC, PVDF, Tygon, and the like. The distal end of aspiration tube 1410 is joined to aspiration port 1404 via methods known in the art including, but not limited to adhesive bonding, ultrasonic welding, overmolding, press fitting, interference fitting, and the like. Aspiration port 1404 may consist of one of any standard connector including, but not limited to luer locks, hose barbs, threaded fittings, etc. and may be fabricated from materials known in the art including, but not limited to nylon, polyurethane, polycarbonate, polyimide, PET, PEEK, polyolefin, PTFE, Pebax, Delrin, polyethylene, stainless steel, nitinol, and combinations thereof. Guidewire retaining valve 1406 may be fabricated from materials including, but not limited to nylon, polyurethane, polycarbonate, polyimide, PET, PEEK, polyolefin, PTFE, Pebax, Delrin, polyethylene, polychloroprene, silicone rubber, nitrile rubber, Viton®, EPDM, butyl rubber, natural rubber, stainless steel, nitinol, and combinations thereof. Guidewire retaining valve enables insertion of an appropriately sized guidewire into the steerable balloon catheter 1400 and maintains the position of the guidewire with respect to shell 1401 when the guidewire is not actively advanced or retracted through the lumen of guidewire retaining valve 1406. In the example shown in FIGS. 14A-14D, the lumen of guidewire retaining valve 1406, the lumen of sliding hub 1409, the guidewire lumen 1418, the lumen of the flexible member 1421, and the lumen of tip 1422 form a continuous path from the proximal end of guidewire retaining valve 1406 to the distal end of tip 1422.

FIG. 14E depicts and alternative embodiment of steerable balloon catheter 1400 may comprising a removable stylet 1423 that is disposed coaxially within the guidewire lumen 1418. The removable stylet may be fabricated from materials known in the art including, but not limited to stainless steel, nitinol, aluminum, titanium, and the like. The removable stylet may be sized such that the distal end of the stylet does not extend past the distal end of tip 1422 when the stylet is fully inserted into guidewire lumen 1418. The proximal end of the stylet may have a feature such as a hook, knob, handle, and the like that provides a location for the user to easily grip the stylet and advance or retract the stylet within the guidewire lumen 1418. The proximal end of the stylet may also comprise a collar, lock, stop, or similar feature that enables operator to insert the stylet into the guidewire lumen until the collar, lock, stop, or similar feature contacts the proximal edge of guidewire retaining valve 1406. The removable stylet may function to increase the rigidity and/or stiffness of the steerable balloon catheter 1400 and allow the distal portion of steerable balloon catheter 1400 to be used to retract or elevate tissue during the course of a surgical procedure. Varying degrees of stiffness or rigidity may be attained by changing the diameter of the stylet, the cross-sectional geometry of the stylet, and the material of the stylet among other variables and/or properties.

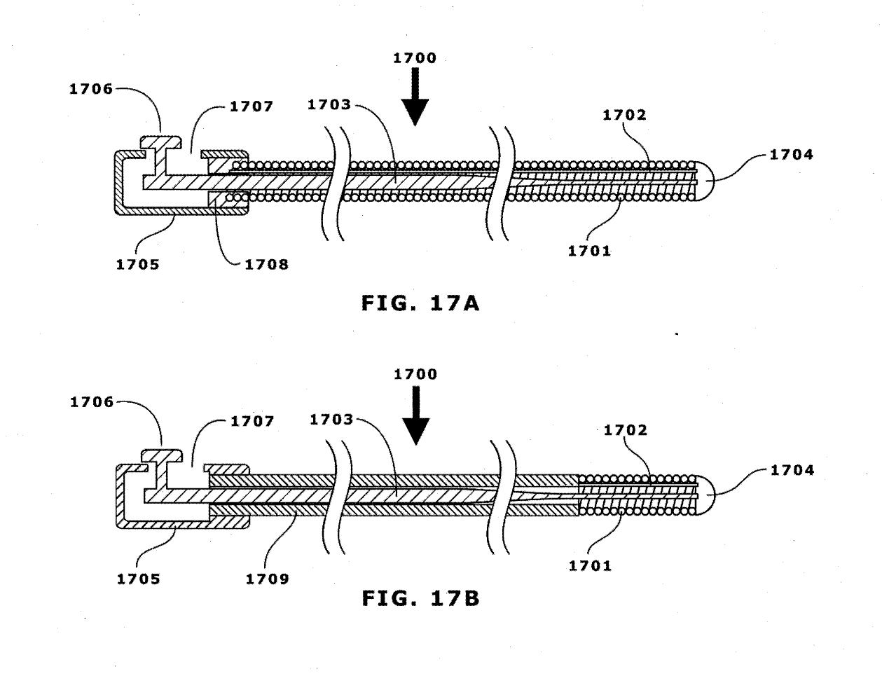

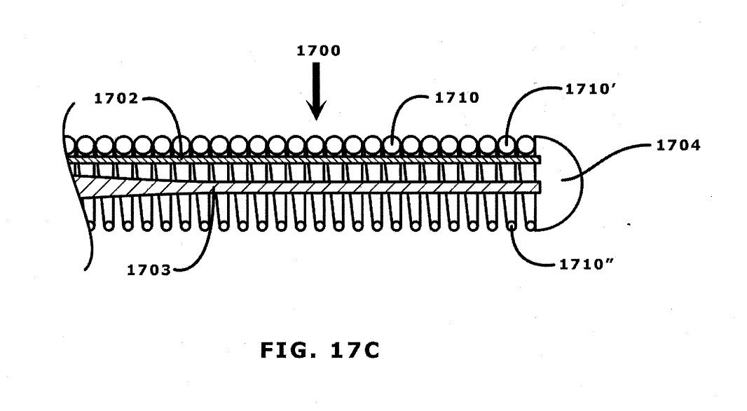

A guidewire (not shown) may be used to facilitate the introduction of the balloon component of the steerable balloon catheter 1400 into a target body lumen, cavity, or ostia. The guidewire may comprise at least one pre-set shape or segment that is less flexible that the remainder of the guidewire. The distal segment of the guidewire may be an atraumatic shape such as a hockey stick, J, or other shape common to interventional cardiology. Alternatively, the guidewire may comprise any of the steerable guidewires disclosed in this patent including those shown in FIGS. 17A-17C. The operator may insert a guidewire such as these into the guidewire lumen 1418 of the steerable balloon catheter 1400 in a state wherein the guidewire is substantially flexible. The guidewire may be advanced through guidewire lumen 1418 and into and/or through the target body cavity, lumen, or ostia. If necessary, the operator may use the steering features of the guidewire to deflect the distal tip of the guidewire and aid in the correct placement of the guidewire with respect to the target anatomy. After the guidewire has been placed in the desired position, the operator may choose to lock the guidewire while the distal tip of the guidewire is in the deflected position. The now-substantially rigid guidewire can now serve as a rail for the steerable balloon catheter to advance distally over and into and/or through the target cavity, lumen, or ostia.

An operator can advance the distal end of the steerable balloon catheter 1400 (with or without the stylet, as desired) into the body of a patient and position the tip 1422 at and/or near the opening of a target body lumen and/or ostium. If a stylet had been used during the positioning step, the operator may then remove the stylet from the steerable balloon catheter 1400. The operator may rotate control knob 1402 to adjust the angle of tip 1422 to the desired orientation and insert an appropriately sized guidewire through the lumen of guidewire retaining valve 1406, lumen of sliding hub 1409, guidewire lumen 1418, lumen of flexible member 1421, and the lumen of tip 1422 into and/or through the target body lumen and/or ostium. The tip 1422 may optionally be returned to a neutral position by rotating control knob 1402 in the opposite direction (until the indicator line on control knob 1402 aligns with the 0 degree marking on shell 1401). The operator can then grasp flange 1407 and control knob 1402 and advance control hub 1402 towards the distal end of window 1417, translating the balloon 1420 distally over the guidewire and into and/or through the target body lumen and/or ostium. The arrangement of sliding hub 1409, guidewire retaining valve 1406, and aspiration seal 1408 ensures that balloon hub 1416 can slide distally inside shell 1401 while maintaining the guidewire in a fixed position relative to shell 1401, balloon hub 1416, and balloon 1420. Similarly, the length of aspiration tube 1410 and inflation tube 1411 allow maintenance of fluid and/or air paths as balloon hub 1416 is advanced distally with respect to shell 1401 and the inserted guidewire. The balloon 1420 may be inflated by introducing fluid and/or air into the balloon hub 1416 via inflation tube 1411 and inflation port 1405 to dilate and treat the body lumen and/or ostium. The balloon 1420 may then be deflated by introducing negative pressure to balloon hub 1416 via inflation tube 1411 and inflation port 1405. The operator may then retract control knob 1402 to the distal end of window 1417 to retract balloon 1420 out of the target body lumen and/or ostium. The guidewire may then be retracted out of the target body lumen and/or ostium and the steerable balloon catheter may be advanced to an additional target body lumen and/or ostium. Optionally, the stylet may be inserted into the guidewire lumen 1418 of the steerable balloon catheter prior to advancing to an additional target body lumen and/or ostium.

FIGS. 14F and 14G depict an alternative configuration of steerable balloon catheter 1400 in which a slider 1424 has been incorporated into balloon hub 1416. In addition to the components and features previously described, shell 1401 further comprises a proximal flange 1407′. While depicted as a flange, feature 1407′ may alternatively comprise at least one ring, grip, indentation, wing, or other structure that may be used with slider 1424 to ease one-handed advancement or retraction of balloon shell 1416 with respect to shell 1401. One method in which this may be accomplished is by placing the thumb within ring 1424, curling the forefinger around flange 1407, and pinching the thumb and forefinger together to advance balloon hub 1416 distally with respect to shell 1401. Conversely, curling the thumb around flange 1407′, placing the forefinger within ring 1424, and pinching the thumb and forefinger together may retract balloon hub 1416 proximally with respect to shell 1401.

Alternatively, steerable balloon catheter 1400 may be fabricated without shell 1401 as shown in FIG. 14H. In this embodiment, the balloon hub 1416 incorporates the features of shell 1401, including aspiration port 1404, aspiration tube 1410, inflation port 1405, and flexible handle extension 1403. This embodiment would include a contort knob (not shown) that functions in a similar fashion to control knob 1402 (shown in FIG. 14A). Control knob 1402 may have reference markings or indicators inscribed or otherwise affixed to the surface or edge of the control knob 1402 that relay information to the user about the angle of tip 1422 (shown in FIG. 14D) with respect to the longitudinal axis of the multi-lumen tubing 1414. For example, the control knob 1402 (shown in FIG. 14A) may have a reference line, dot, or other indicator inscribed or otherwise applied to its surface. Corresponding markings that indicate tip angles of 0 degrees, 70 degrees, 90 degrees and 110 degrees, for example, may be inscribed, engraved, pad printed, or otherwise applied to the outer surface of balloon hub 1416. Alignment of the reference mark on the control knob 1402 with the desired tip angle marking would produce the corresponding angle between the tip 1422 (shown in FIG. 14D) and the longitudinal axis of the multi-lumen tubing 1414. In another example (not shown), the control knob 1402 may have reference markings or indicators inscribed or otherwise affixed to the surface or edge of the control knob 1402 that relay information to the user about the angle of tip 1422 with respect to the longitudinal axis of multi-lumen tubing 1414. For example, the control knob 1402 may have markings that indicate tip angles of 0 degrees, 70 degrees, 90 degrees and 110 degrees. These markings may be referenced against a line, dot, or other indicator inscribed or otherwise applied to the outer surface of balloon hub 1416. All other components and variations are as previously described for FIGS. 14A-14E.

An operator can advance the distal end of the steerable balloon catheter 1400 shown in FIG. 14H (with or without the stylet, as desired) into the body of a patient and position the tip 1422 at and/or near the opening of a target body lumen and/or ostium. If a stylet had been used during the positioning step, the operator may then remove the stylet from the guidewire lumen 1418 of steerable balloon catheter 1400. The operator may rotate control knob 1402 to adjust the angle of tip 1422 to the desired orientation and insert an appropriately sized guidewire through guidewire lumen 1418, lumen of flexible member 1421, and the lumen of tip 1422 into and/or through the target body lumen and/or ostium. The tip 1422 may optionally be returned to a neutral (approximately zero degrees) position by rotating control knob 1402 in the opposite direction (until the indicator line on control knob 1402 aligns with the 0 degree marking on the outer surface of balloon hub shell 1416). The operator can then translate steerable balloon catheter 1400 distally over the guidewire such that balloon 1420 is positioned into and/or through the target body lumen and/or ostium. Ideally, the guidewire should be maintained in a fixed position relative to the target body lumen and/or ostium during this translation step of the procedure. The balloon 1420 may be inflated by introducing fluid and/or air into the balloon hub 1416 via inflation port 1405 to dilate and treat the body lumen and/or ostium. The balloon 1420 may then be deflated by introducing negative pressure to balloon hub 1416 via inflation port 1405. The operator may then retract steerable balloon catheter 1400 to retract balloon 1420 out of the target body lumen and/or ostium. The guidewire may then be removed from the target body lumen and/or ostium and the steerable balloon catheter may be advanced to an additional target body lumen and/or ostium. Optionally, the stylet may be re-inserted into the guidewire lumen 1418 of the steerable balloon catheter 1400 prior to advancing to an additional target body lumen and/or ostium.

One embodiment of a handle 1425 that may be incorporated into steerable balloon catheter 1400 is shown in FIG. 14I. Handle 1425 may be fabricated using methods known in the art including, but not limited to machining, molding, stereolithography, and the like from materials known in the art including PMMA, polycarbonate, Pebax, nylon, ABS, stainless steel, aluminum, anodized aluminum, titanium, and the like. Handle 1425 may be axisymmetric, non-axisymmetric, straight, curved, bilaterally symmetric about any plane, bilaterally asymmetric about any plane, or any other shape that permits handling of steerable balloon catheter 1400. Handle 1425 is connected to steerable balloon catheter 1400 via flexible handle extension 1403; handle 1425 and flexible handle extension may be joined using methods known in the art including, but not limited to threading and tapping, use of a set screw, press fitting, adhesive bonding, heat fusing, ultrasonic welding, overmolding, and the like. Handle 1425 further comprises at least one grip 1426 that facilitates handling and or comfort during the course of a medical procedure (e.g. to ease holding the handle with hand or finger tips while also manipulating an adjacent endoscope). Grip 1426 may be concave, convex, or a complex shape and/or surface that is suitable for providing traction and comfort to the user. Grip 1426 may be machined into or onto handle 1425 as a second operation, incorporated into handle 1425 during a molding or overmolding process, or fabricated using other techniques known to those of skill in the art. Grip 1426 may further comprise a material that is softer than that of the rest of handle 1425; the softer material may include, but is not limited to, Pebax, polyurethane, polyethylene, polychloroprene, silicone rubber, nitrile rubber, Viton®, EPDM, butyl rubber, natural rubber, and the like and may be joined to handle 1425 using methods known in the art including, but not limited to adhesive bonding, ultrasonic welding, overmolding, heat fusing, and the like. It is obvious that handle 1425 of 14I can be used with any of the catheter and device embodiments of this invention and is not limited to the steerable balloon catheter embodiments described here.

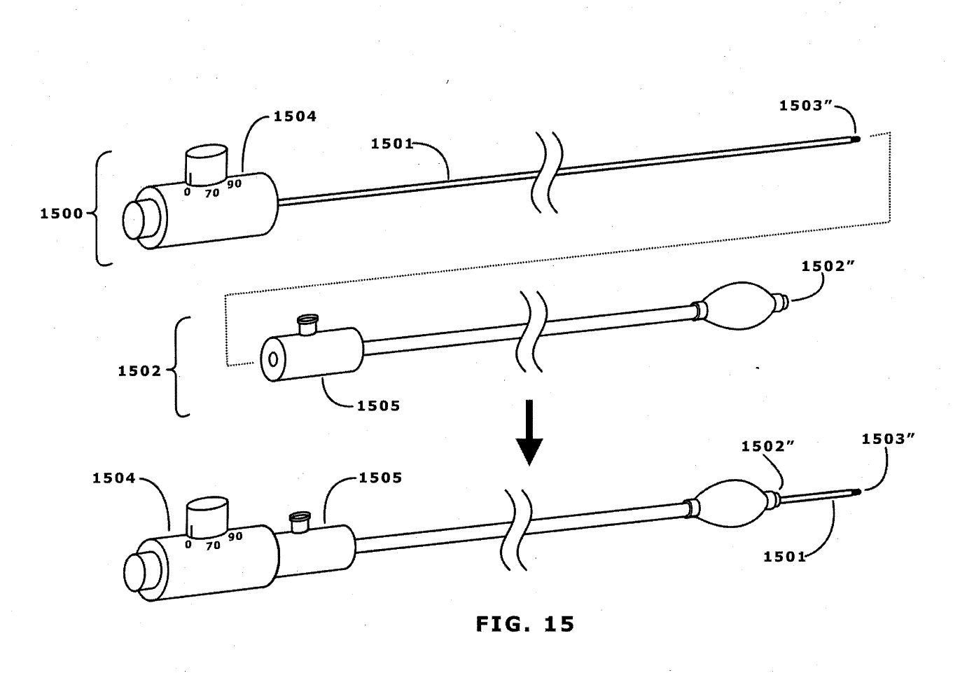

FIG. 15 depicts another embodiment of the steerable guide system 1500 wherein the outer diameter of the cannula 1501 is sized to fit within the lumen of an over the wire balloon catheter 1502. The over the wire balloon catheter 1502 may be of the design disclosed in co-pending U.S. Pat. App. No. 61/352,244 herein incorporated in full by reference. The transport member 1503″ may be a pre-shaped coiled guidewire or pre-shaped mandrel of materials that include but are not limited to stainless steel, nitinol, nylon, PET, polycarbonate, PEBAX, HDPE, polyurethanes, fluoropolymers, composite materials such as PEBAX tubing with embedded braids of nitinol, stainless steel, copper, and the like. The length of the cannula 1501 and the transport member 1503″ may be larger than the overall length of the balloon catheter 1502 such that the distal tip of the transport member 1503″ extends beyond the distal tip of the balloon catheter 1502″. The steerable guide system cannula hub 1504 may be configured to reversibly connect with the balloon catheter hub 1505 such that the steerable guide system 1500 may be inserted into the over the wire balloon catheter 1502 and reversibly lock the cannula hub 1504 to the balloon catheter hub 1505, thus enabling an operator to use the combined devices as a single unit. The releasable connection may be achieved through the use of mechanisms that include but are not limited to living hinges, magnets, detents, spring and levers, spring and balls, rotating collars or collets, key and keyhole mechanisms, screws and taps, compliant or semicompliant rings or gaskets, and the like.

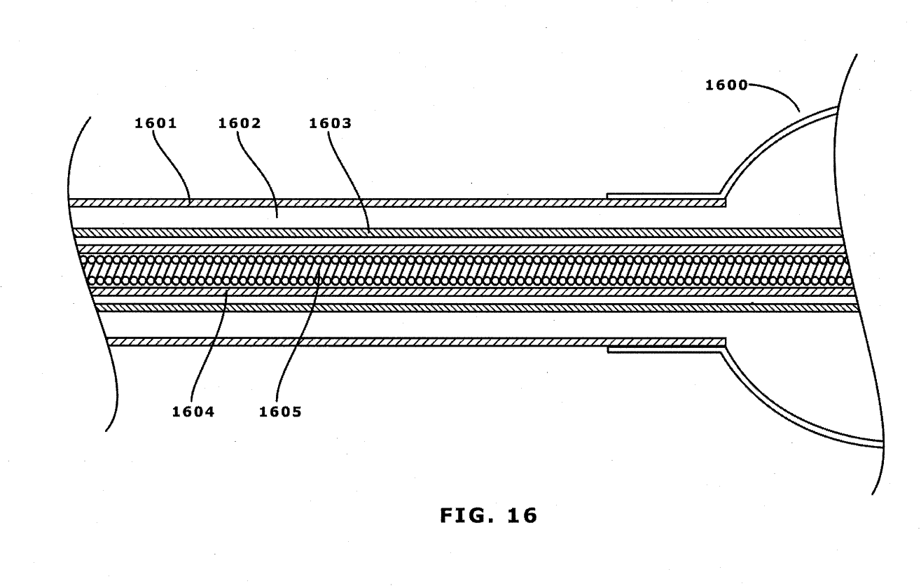

FIG. 16 depicts a cross-sectional view of the steerable guide system 1500 inserted into an over the wire balloon catheter 1502. The balloon catheter 1502 in this figure comprises an expandable balloon segment 1600, a catheter shaft 1601, and an inner lumen 1602 defined by an internal elongate member 1603. The expandable balloon segment 1600 is in fluid communication with the inner lumen 1602 between the catheter shaft 1601 and the internal elongate member 1603. After insertion into balloon catheter 1502, the steerable guide system 1500 resides in the inner lumen 1602. The cannula 1604 is sized to be slidably disposed within lumen 1602. As described previously, transport member 1605 is slidably disposed within cannula 1604. The relative linear and rotational motion of cannula 1604 with respect to transport member 1605 serves to adjust the angle of the transport member tip (not shown) with the longitudinal axis of the transport member 1605 and the rotational orientation of the transport member 1605 with respect to the cannula 1604. In this example, transport member 1605 comprises a coiled guidewire or other mandrel.

FIGS. 17A through 17C depict three exemplary embodiments of a steerable guide system that employs a steerable guidewire. In FIG. 17A, steerable guidewire 1700 comprises a coil 1701, stiffening member 1702, and corewire 1703 attached to atraumatic tip 1704 on their respective distal ends. Coil 1701 and stiffening member 1702 are attached to retaining collar 1708 at their respective proximal end. Attachment methods may include, but is not limited to welding, ultrasonic welding, soldering, adhesive bonding, swaging, or combinations thereof. Coil 1701, stiffening member 1702, and corewire 1703 may be fabricated from materials known in the art including, but not limited to stainless steel, nitinol, platinum, titanium, gold, or any metal. Stiffening member 1702 runs through the lumen of coil 1701 and is of sufficient rigidity to prevent the coil 1701 from stretching at points close to the stiffening member 1702 when the coil 1701 is placed under tension. Retaining collar 1708 is attached to housing 1705, which comprises a channel or groove 1707. Corewire 1703 is positioned within the lumen of coil 1701 and the proximal section of corewire 1703 passes through the lumen of retaining collar 1708 terminating within the lumen of housing 1705. Corewire 1703 is connected to slide 1706 through the channel or groove 1707 in housing 1705. The distal section of corewire 1703 may assume a cylindrical cross-section, or it may flatten or be formed into any desired cross-section. Advancing or pushing slide 1706 in the distal direction forces the distal section of coil 1701 to assume a bent or curved shape. In the case of a corewire 1703 comprising a flattened distal section, the direction of the bend will be influenced by the orientation of the long axis of the cross-section. The coiled wire will preferentially bend in a direction that is approximately orthogonal to the long axis of the cross-section of the distal section of corewire 1703. The extent of the bend, and the location of the beginning of the bend, is dictated by the location, length and magnitude of the taper on corewire 1703 as well as the rigidity of stiffening member 1702.

The steerable guidewire depicted in FIG. 17B is similar to the steerable guidewire shown in FIG. 17A, however, retaining collar 1708 has been replaced with a rigid elongate member 1709. Rigid elongate member 1709 has a lumen running throughout its length and is bonded to housing 1705 at its proximal end and is bonded to coil 1701 and stiffening member 1702 at its distal end. Rigid elongate member 1709 may be fabricated from materials including, but not limited to stainless steel, nitinol, nylon, PET, polycarbonate, PEBAX, HDPE, polyurethanes, fluoropolymers, composite materials such as PEBAX tubing with embedded braids of nitinol, stainless steel, copper, and the like.

FIG. 17C illustrates another variation of the steerable guidewire of FIGS. 17A and 17B. In this embodiment, the distal portion of steerable guidewire 1700 comprises an atraumatic tip 1704 bonded to the distal ends of stiffening member 1702, tapered wire 1703, and coil 1710. Coil 1710 has been fabricated to have coils of smaller diameter 1710″ on a fraction of the perimeter or circumference of the coiled wire. FIG. 17C shows a configuration in which wire comprising coil 1710 has maximum diameters 1710′ and a minimum diameters 1710″ spaced so that they are on opposite sides of finished coiled. Coil 1710 may be fabricated by profile grinding the wire prior to the coil winding operation in a wave shape, laser cutting a wave shape into the wire prior to or after the coil winding operation, or other techniques known in the art. A wave shape is portrayed in this example, however, it should be apparent to one of skill in the art that other wire profiles may be generated that will produce different bending and/or steering tendencies in the finished coil 1710. Alternatively (not shown), the configuration can be modified to form a bend when the corewire 1703 is pulled proximally. In this configuration, the slide 1706 is initially positioned at the distal end of the groove or channel 1707. As slide 1706 is translated or pulled proximally, tension is placed on corewire 1703 and its connections causing the assembly to bend. Optionally (not shown), any of the versions of guidewire 1700 shown in FIGS. 17A-17C may comprise at least one marker suitable for use in a respective visualization or navigation system. For example, a radio-opaque segment or band may be incorporated into guidewire 1700 to enable or improve visualization in a fluoroscopic visualization system. As another example, an electromagnetic beacon may be incorporated into guidewire 1700 to enable or improve visualization and/or localization of the guidewire with an electromagnetic navigation system such as the Fusion™ ENT Navigation System (Medtronic Xomed, Jacksonville, Fla.) or the i-Logic™ System (superDimension, MN). Alternatively, guidewire 1700 may comprise magnetic guidance features such as those described in co-pending U.S. Pat. App. No. 61/366,676, herein incorporated in full by reference. While these examples illustrate the use of the guidewires 1700 with specific image guidance systems, it should be apparent to one of skill in the art that the guidewires 1700 could be appropriately modified to function in concert with a wide range of image guidance systems employing modalities including, but not limited to computed tomography, infrared, magnetic resonance, or ultrasound.

While the guidewires 1700 shown in FIGS. 17A-17C depict a design that enables the distal end of the guidewire to assume a shape from a continuous range of potential shapes (e.g. a curve with any angle from 0 to 150 degrees), the guidewires 1700 may be configured to enable a discrete change in shape (e.g. a curve of 0, 70, or 150 degrees). For example, the housing 1705 shown in FIGS. 17A and 17B may comprise a channel or groove 1707 that is similar to keyway 905 shown in FIGS. 9A and 9B. The slide 1706 may engage one of the individual slots in groove 1707 that corresponds to a specific angle of deflection of the distal section of coil 1701. For example, a channel or groove 1706 comprising one individual slot would enable the user to position the device in either an active or passive state. The passive state (e.g. a 0 degree angle of deflection of the distal section of coil 1701) would be obtained by placing the slide 1706 out of the individual slot of channel or groove 1707. The active state (e.g. a 150 degree angle of deflection of the distal section of coil 1701) would be obtained by positioning the slide 1706 within the individual slot of channel or groove 1707. Although a key and keyway mechanism is described as an exemplary design for enabling a discrete selection of the state of the guidewires 1700, it should be obvious to those of skill in the art that equivalent control mechanisms including, but not limited to detents, living hinges, spring, ball, and detent arrangements, winch mechanisms, combinations thereof, and the like may be employed to achieve similar functionality. Additional parameters such as stiffness may be controlled in a similar manner. Furthermore, the parameters of interest (e.g. shape, stiffness, etc.) may be controlled over one or more segments of the guidewires 1700.

Alternatively (not shown), the devices of the invention may comprise a guidewire with an expandable distal segment. The expandable segment may be an inflatable balloon, a strut or stent-like structure, a hook, crossbar, spiral, or any feature that may be inserted through a target body lumen and/or ostium in a narrow configuration, then activated to expand to a size larger than that of the target body lumen and/or ostium. This action would enable the guidewire to maintain position in the target body lumen and/or ostium. For example, a guidewire with an expandable balloon element may be inserted into a target body lumen and/or ostium such that the expandable balloon traverses and exits the target body lumen and/or ostium. The balloon may be expanded to a diameter larger than that of the target body lumen and/or ostium, anchoring the guidewire within the target body lumen and/or ostium. A working device such as a dilation catheter or stent may then be advanced over the guidewire without dislodging the guidewire from the target body lumen and/or ostium. An expandable segment of this nature may further be combined with any of the steerable guidewire designs disclosed herein to create a guidewire that comprises steerable features along with an expandable distal segment. Standard manufacturing and materials used to fabricate medical catheters and wires could be used for the guidewire with expandable distal segment including, but not limited to stainless steel, nitinol, nylon, PET, polycarbonate, PEBAX, HDPE, PMMA, polyurethanes, fluoropolymers, composite materials such as PEBAX tubing with embedded braids of nitinol, stainless steel, copper, and the like.

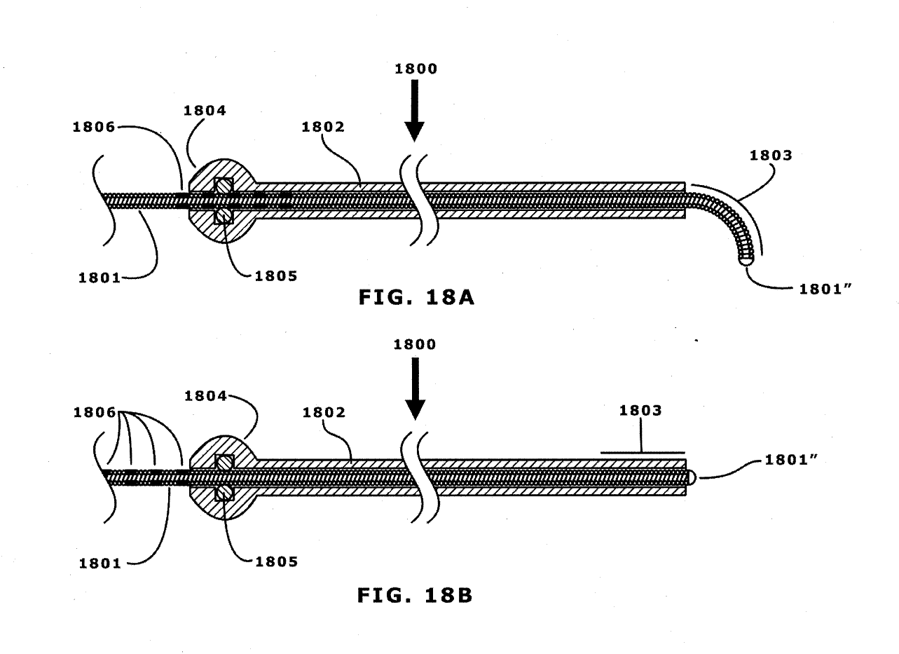

FIGS. 18A and 18B provide cross sectional views of one embodiment of the steerable guide system of the invention 1800 with delineation of the system components. In this figure, system components include transport member 1801 and cannula member 1802. The transport member component 1801 could be comprised of a shapeable guidewire. The distal segment of the transport member, 1803, could be pre-formed in a desired geometric configuration. For example, the substantially distal segment of the transport member 1803 may be pre-formed to position the distal tip 1801″ in a generally orthogonal or ninety (90) degree orientation with respect to the straight segment of transport member 1801 (proximal to the pre-formed segment). Unconstrained, the distal tip 1801″ of the transport member's distal segment 1803 would remain at its generally orthogonal or ninety (90) degree position with respect to the proximal segment of the transport member 1801. Obviously, the guidewire may be pre-shaped to a desired angle other than the ninety (90) degree angle shown in this example. The transport member 1801 could be constructed from semi-rigid to flexible plastics, polymers, metals and composites including braided tubing configurations well known in the art. For example, transport member 1801 could be made from the following non-limiting list of materials: Pebax, nylon, urethane, silicone rubber, latex, polyester, Teflon, Delrin, PEEK, PMMA, stainless steel, nitinol, platinum etc. Permutations of these materials could also be envisioned. The preformed shape could be achieved through a number of processes such as heat setting, molding, shape memory applications with or without nitinol etc.

The cannula member 1802 represents a substantially rigid component of the system that also is comprised of a proximal and distal end with a continuous lumen therethrough. Cannula member 1802 could have a hub 1804, at its proximal end as shown in FIGS. 18A-18B. Hub 1804 serves as an aid to control the steerable guide system. Hubs 1804 could be made from standard metals, plastics, polymers, composites or other materials well known in the art. The process to make hub 1804 may include, but is not limited to well known methods such as injection molding, casting, machining etc. In the embodiment shown in FIGS. 18A-18B, the components are arranged with the cannula 1802 positioned coaxially over the outer surfaces of the transport member 1801. Cannula 1802 would be able to move and/or slide in the longitudinal direction both proximally and distally. Travel would be unconstrained in both the proximal and distal directions and cannula member 1802 could be pushed along the outer wall of transport member 1801 until it was completely removed off transport member 1801 as a free-standing component. Alternatively, transport member 1801 could be inserted into the proximal end of cannula 1802 as part of a pre-procedure preparation step. As shown in FIGS. 18A-18B, as cannula 1802 is advanced distally it captures preformed shape 1803 within its lumen. In doing so, the pre-shaped segment of transport member 1803 assumes a shape that generally mimics the geometry of cannula 1802. Cannula 1802 could be of an overall length that would be less than the overall length of transport member 1801. It would also be ideal if cannula 1802 could slide proximally and distally over adequate length to steer the distal tip of the transport member 1801″ through its range of motion allowing transformation of the transport member 1801 from a substantially straight configuration when constrained by cannula 1802 to its pre-formed geometry as it is unconstrained.

FIGS. 18A-18B depict a retaining member 1805 that acts to hold the position of transport member 1801 with respect to cannula 1802 after the physician operator has released transport member 1801. For example, FIGS. 18A-18B show the retaining member 1805 as an o-ring located in the cannula hub 1804. The o-ring 1805 would apply enough friction to the transport member 1801 to fix the transport member with respect to cannula 1802 after insertion and placement of transport member 1801. While depicted as an o-ring in FIGS. 18A-18B, retaining member 1805 could be any design, component, or feature known in the art that can act to fix transport member 1801 with respect to cannula 1802. This includes, but is not limited to Touhy-Borst valves, clips, detents, lumen narrowing, springs, levers, living hinges, irises, and the like. Though shown in cannula hub 1804 in FIGS. 18A-18B, retaining member 1805 may also be located at any position within cannula 1802. Furthermore, multiple retaining members 1805 of varied designs may be incorporated into steerable guide system 1800. As noted for other embodiments of the invention, markings or other indicators may be placed on, etched into, or otherwise applied to the transport member 1801 to indicate the shape of the pre-shaped segment of the transport member 1803. For example, FIGS. 18A-18B show markings 1806 that may be referenced against the proximal edge of cannula hub 1804 to relay information to the user about the shape of distal segment 1803 of transport member 1801.

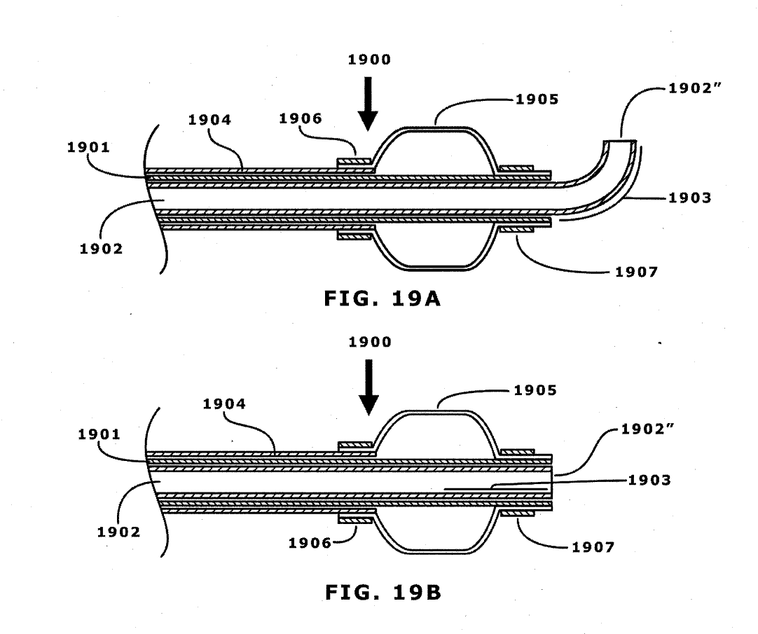

FIGS. 19A and 19B provide cross sectional views of one embodiment of the steerable guide system of the invention 1900 with delineation of the system components. In this figure, system components include cannula member 1901 which acts as the inner member of the balloon and has been fitted with a catheter shaft 1904 and an expandable balloon 1905, transport member 1902, proximal marker band 1906, and distal marker 1907. The distal segment of the transport member 1903, could be pre-formed in a desired geometric configuration. For example, the substantially distal segment of the transport member 1902 may be pre-formed to position the distal tip 1902″ in a generally orthogonal or ninety (90) degree orientation with respect to the straight segment of transport member 1902 (proximal to the pre-formed segment). Unconstrained, the distal tip 1902″ of the transport member's distal segment 1903 would remain at its generally orthogonal or ninety (90) degree position with respect to the proximal segment of the transport member 1902. The transport member 1902 could be constructed from semi-rigid to flexible plastics, polymers, metals and composites including braided tubing configurations well known in the art. For example, transport member 1902 could be made from the following non-limiting list of materials: Pebax, nylon, urethane, silicone rubber, latex, polyester, Teflon, Delrin, PEEK, stainless steel, nitinol, platinum etc. Furthermore, transport member 1902 may be reinforced with braids, coils, laminates, and the like well known in the art. Permutations of these materials could also be envisioned. The preformed shape could be achieved through a number of processes including, but not limited to heat setting, molding, shape memory applications with or without nitinol and the like.

The cannula member 1901 represents a flexible to substantially rigid component of the system that is compromised of a proximal and distal end with a continuous lumen therethrough. In the embodiment shown in FIGS. 19A-19B, the components are arranged with the cannula 1901 positioned coaxially over the outer surfaces of the transport member 1902. Cannula 1901 would be able to move and/or slide in the longitudinal direction both proximally and distally. Travel would be unconstrained in both the proximal and distal directions and cannula member 1901 could be pushed along the outer wall of transport member 1902 until it was completely removed off transport member 1902 as a free-standing component. As shown in FIGS. 19A-19B, as cannula 1901 is advanced distally it captures preformed shape 1903 within its lumen. In doing so, the pre-shaped segment of transport member 1902 assumes a shape that generally mimics the geometry of cannula 1901. Cannula 1901 could be of an overall length that would be less than the overall length of transport member 1902. It would also be ideal if cannula 1901 could slide proximally and distally over adequate length to steer the distal tip of the transport member 1902″ through its range of motion allowing transformation of the transport member 1902 from a substantially straight configuration when constrained by cannula 1901 to its pre-formed geometry as it is unconstrained.

Marker bands 1906 and 1907 are located proximal and distal to balloon 1905, and provide a means to ascertain the position of balloon 1905 with respect to the anatomy of interest. The marker bands may be chosen for visibility in a particular imaging system. For example, the bands may be pad printed markings when a visible light system such as an endoscope is used for visualization of the procedure. The marker bands may also be collars or rings of a material that is dyed to a color that can be differentiated from that of the balloon and/or the catheter shaft 1904 and/or the cannula 1901. In this example, the bands may be fabricated from materials such as, but not limited to, polycarbonate, polyimide, Pebax, nylon, polyurethane, PET, PEEK, polyethylene, shrink tubing, and the like. In another example, the bands may be platinum, gold, platinum/iridium or other radiopaque materials if fluoroscopy (for example) is used as the method of visualization during the procedure. Alternatively (not shown), marker bands 1906 and 1907 could be placed on the cannula 1901 underneath or within balloon 1905. Extension of this concept to other image guidance systems that utilize modalities including, but not limited to magnetic, electromagnetic, computed tomography, infrared, magnetic resonance, or ultrasound should be readily apparent to one of skill in the art.

Cannula member 1901 and catheter shaft 1904 provide a lumen for fluid and/or air to communicate with expandable balloon 1905. The lumen may be in communication with a port located proximal to the balloon (not shown) that allows for introduction of positive or negative pressure into the lumen and expandable balloon 1905. The port may comprise a male or female luer lock, a male or female luer, an extension line, a hose barb, or other such features well known in the art for the inflation or deflation of a balloon used in medical procedures. Expandable balloon 1905 may be bonded to cannula member 1901 and catheter shaft 1904 using methods common in the art, including, but not limited to ultrasonic welding, adhesive bonding, heat fusing, swaging, crimping, and the like. The cannula member, catheter shaft, and expandable balloon may be of the design disclosed in co-pending U.S. Pat. App. No. 61/352,244 herein incorporated in full by reference.

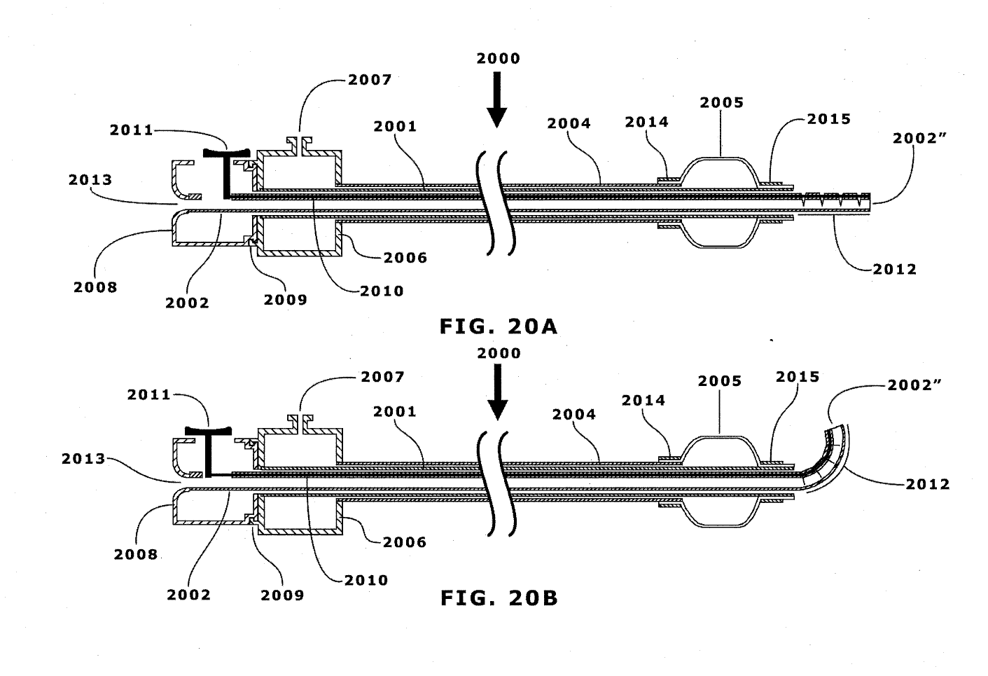

FIGS. 20A and 20B illustrate another embodiment of the invention 2000 comprising cannula member 2001 that has been fitted with a catheter shaft 2004 and an expandable balloon 2005. The lumen between cannula member 2001 and catheter shaft 2004 provide for fluid and/or air communication between pressure chamber 2006 and expandable balloon 2005. Expandable balloon 2005 may be bonded to cannula member 2001 and catheter shaft 2004 using methods common in the art, including, but not limited to ultrasonic welding, adhesive bonding, heat fusing, swaging, crimping, and the like. The cannula member, catheter shaft, and expandable balloon may be of the design disclosed in co-pending U.S. Pat. App. No. 61/352,244 herein incorporated in full by reference.

Marker bands 2014 and 2015 are located proximal and distal to balloon 2005, and provide a means to ascertain the position of balloon 2005 with respect to the anatomy of interest. The materials characteristics of marker bands 2014 and 2015 may be chosen for visibility in a particular imaging system. For example, the bands may be pad printed markings when a visible light system such as an endoscope is used for visualization of the procedure. Marker bands 2014 and 2015 may also be collars or rings of a material that is dyed to a color that can be differentiated from that of the balloon and/or the catheter shaft 2004 and/or the cannula 2001. In this example, the bands may be fabricated from materials such as, but not limited to, polycarbonate, polyimide, Pebax, nylon, polyurethane, PET, PEEK, polyethylene, shrink tubing, and the like. In another example, marker bands 2014 and 2015 may be platinum, gold, platinum/iridium or other radiopaque materials if, for example, fluoroscopy is used as the method of visualization during the procedure. Extension of this concept to other materials and visualization methodologies including, but not limited to magnetic modalities, ultrasound, electromagnetic navigation, infrared navigation, computed tomography, and the like should be readily apparent to one of skill in the art.

Pressure chamber 2006 comprises a port 2007 for inflation or deflation of balloon 2005. Port 2007 may comprise a male or female luer lock, a male or female luer, a hose barb, an extension line, or other such features known in the art for the inflation or deflation of a balloon used in medical procedures. The proximal wall of pressure chamber 2006 is connected to proximal hub 2008 in such a manner that proximal hub 2008 can rotate with respect pressure chamber 2006. This may be achieved through the use of a ridge and groove mechanism 2009 as shown in FIGS. 20A and 20B, or through other methods or mechanisms known in the art. Proximal hub 2008 comprises a transport member 2002, a tension wire 2010, and an actuator 2011. The transport member 2002 in the example is a dual lumen tube with tension wire 2010 running through one of the lumens and bonded to the distal end of transport member 2002. The distal segment of transport member 2012 has several segments of tubing removed; these segments may be square cut, chevron cut, or other geometries that allow the distal segment 2012 to flex when the distal tip of the transport member 2002″ is placed in tension. The distal segment 2012 may be cut using methods known in the art including, but not limited to laser cutting, EDM, and the like. Alternatively, distal segment 2012 (not shown) may comprise the previously disclosed designs and methods of shaping distal segment 2012. The proximal end of tension wire 2010 is connected to actuator 2011 through a channel, groove, window, or other feature in proximal hub 2008. The proximal end of transport member 2002 is mated to a window, hole, recess, or other gap or void 2013 in proximal hub 2008 that allows access to the lumen of transport member 2002. Feature 2013 may include inward sloping walls as shown in FIGS. 20A-20B that ease insertion of guidewires or other operating instruments into the lumen of transport member 2002.

Cannula member 2001 is arranged coaxially over transport member 2002. Retraction of actuator 2011 in the proximal direction places a load on tension wire 2010, which in turn pulls on the distal end of transport member 2002″. The tensile load on the distal end of transport member 2002″ collapses the distal segment of transport member 2012 to a degree dictated by the geometry of the segments removed from the transport member 2002 and the amount of tension placed on tension wire 2010. The rotational orientation of the distal tip of transport member 2002″ may be adjusted by rotating proximal hub 2008 with respect to pressure chamber 2006. While this example illustrates the use of a tension wire 2010 to pull on the distal end of the transport member 2002″ to induce a change in the shape of the distal segment of the transport member 2012, this does not preclude the use of other methods of inducing a change in the distal segment of the transport member. These methods include, but are not limited to a pushing on a stiff wire bonded to the distal end of the transport member, use of a shape memory material such as nitinol to directly or indirectly change the shape of the distal end of the transport member (e.g. via temperature change as a result of passage of electrical current through the shape memory material, via a change in length of the tension wire as a result of a temperature change, etc.), and others known in the art.

Similarly, while actuator 2011 is illustrated as a slide mechanism in FIGS. 20A-20B, other mechanisms known in the art for placing tension on a wire are suitable as well. This includes, but is not limited to gearing or ratcheting mechanisms, screw mechanisms, lever mechanisms, winch mechanisms, and the like.

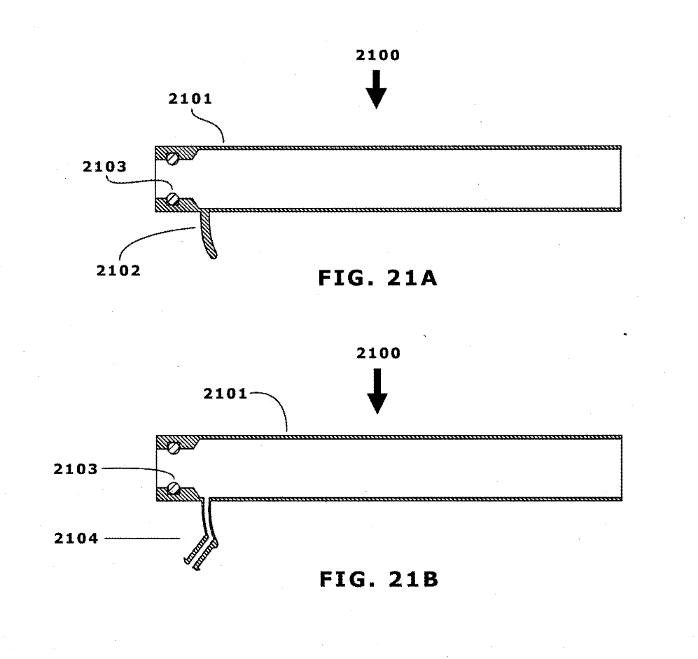

FIGS. 21A and 21B depict a telescoping sheath 2100 that may be a component of any of the devices of the invention described herein. For example, telescoping sheath 2100 may be coaxially arranged over the balloon shaft 1412 to provide protection to balloon 1420 during insertion of the steerable balloon catheter 1400 into a patient. Telescoping sheath 2100 comprises an elongate member 2101 with proximal and distal ends and a lumen running therethrough. FIG. 21A depicts one example of telescoping sheath 2100 that further comprises a seal 2103 and a grip 2102. Seal 2103 may be an o-ring, gasket, or other material fabricated from materials known in the art including, but not limited to polyethylene, polychloroprene, silicone rubber, nitrile rubber, Viton®, EPDM, butyl rubber, natural rubber, and the like. While seal 2103 is depicted as an o-ring or gasket in FIGS. 21A and 21B, it may also comprise components including, but not limited to a Touhy-Borst valve, living hinge, iris valve, clamp, chuck, or combination thereof. Grip 2102 is shown as a flange in FIGS. 21A and 21B, however, grip 2102 may comprise geometries including, but not limited to at least one ring, indentation, wing, or other structure. FIG. 21B depicts an alternative example of telescoping sheath 2100 that replaces grip 2102 with aspiration port 2104. If telescoping sheath 2100 is arranged over a mandrel or shaft (not shown) such that seal 2103 provides an fluid and/or air tight seal against the mandrel or shaft, a vacuum applied to aspiration port will enable aspiration or suction to be applied from the distal end of telescoping sheath 2100. Telescoping sheath 2100 may be incorporated in any of the devices of the invention for purposes including, but not limited to increasing the lubricity of the device, reducing the rigidity of one or more tissue-contacting surfaces of the device, increasing the stiffness of one or more sections of device, providing a pathway for aspiration or sampling of body fluids or tissues, providing a marker that enables use in a given visualization system (magnetic, fluoroscopy, electromagnetic navigation systems, ultrasound, infrared navigation systems, computed tomography, and the like), protecting the dilation element during transit to the treatment area, enabling retraction of tissues, and combinations thereof.

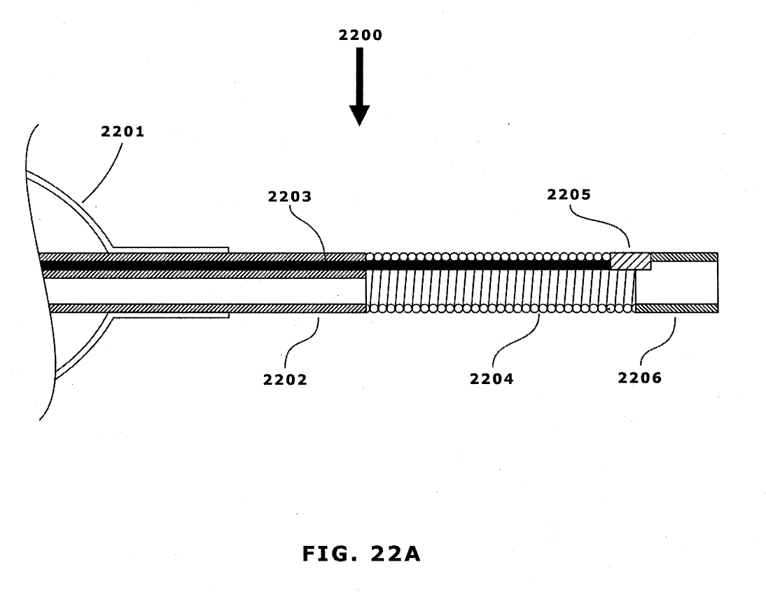

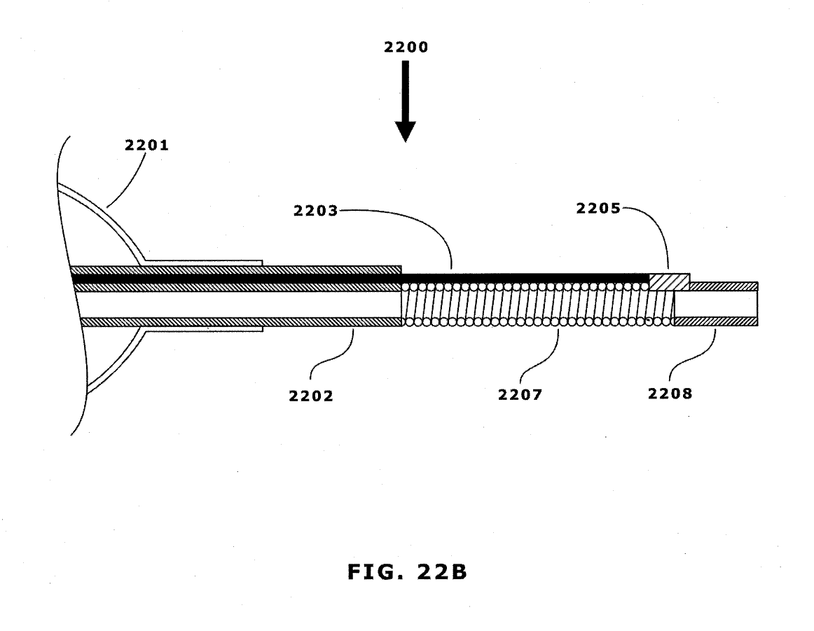

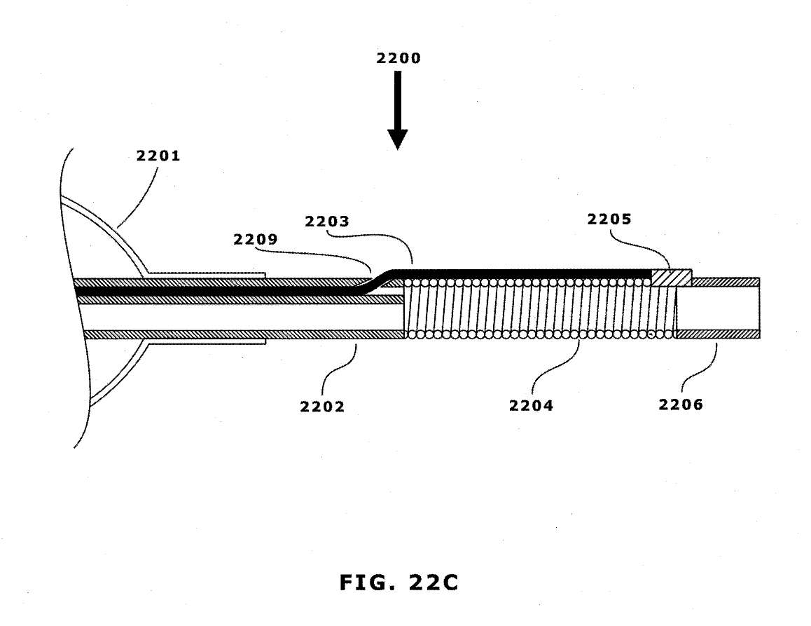

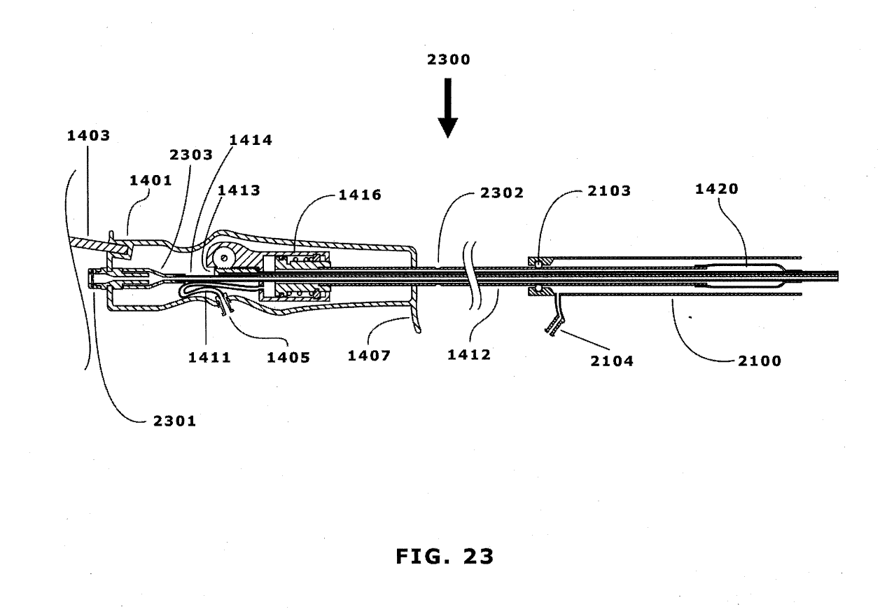

FIGS. 22A-22C depict several embodiments of the distal ends of steerable balloon catheters and catheter systems such as those described in FIGS. 14A-14D and 23. FIG. 22A depicts one example of the distal end of a steerable balloon catheter 2200 comprising a balloon 2201 bonded to the outer surface of a multi-lumen tube 2202. The multi-lumen tube 2202 is depicted as having two lumens, however, it should be obvious to one of skill in the art that additional lumens may be present in this component. Multi-lumen tube 2202 may be fabricated from materials known in the art including, but not limited to nylon, polyurethane, polycarbonate, polyimide, PET, PEEK, polyolefin, PTFE, Pebax, Delrin, polyethylene, stainless steel, nitinol, and combinations thereof. The distal end of multi-lumen tube 2202 is joined to the proximal end of flexible member 2204 using techniques known in the art including, but not limited to heat fusing, adhesive bonding, ultrasonic welding, interference fitting, threading, press fitting, crimping, and combinations thereof. Flexible member 2204 may be a coiled wire fabricated from materials including, but not limited to stainless steel, nitinol, nylon, PET, polycarbonate, PEBAX, HDPE, polyurethanes, fluoropolymers, composite materials such as PEBAX tubing with embedded braids of nitinol, stainless steel, copper, and the like. The distal end of flexible member 2204 is joined to the proximal end of tip 2206 using techniques known in the art including, but not limited to heat fusing, adhesive bonding, ultrasonic welding, interference fitting, threading, press fitting, crimping, and combinations thereof. Tip 2206 comprises an elongate member with at least one lumen extending from its proximal to distal ends. Tip 2206 may be fabricated from soft and/or flexible materials known in the art including, but not limited to polyurethane, Pebax, silicone rubber, polyethylene, etc. The distal end of tip 2206 may be shaped into an atraumatic geometry such as but not limited to a taper, hemisphere, ball, and the like. The physical characteristics and geometry of the tip 2206 may be uniform or variable over its length. Pullwire 2203 resides within one of the lumens of multi-lumen tube 2202, runs through the lumen of flexible member 2204, and is joined to the distal end of flexible member 2204 via bond 2205. Bond 2205 may be realized through techniques known in the art including, but not limited to welding, adhesive bonding, crimping, and the like. Additionally, the distal end of steerable balloon catheter 2200 may comprise (not shown) marker bands or beacons that allow for visualization of the device using methods known in the art including, but not limited to magnetic modalities, ultrasound, infrared navigation systems, electromagnetic navigation systems, computed tomography, fluoroscopy, and the like.

The embodiment of the distal end of combined steerable guide/dilation device 2200 depicted in FIG. 22B is similar to that shown in FIG. 22A, however, the diameter of flexible member 2207 is reduced such that pullwire 2203 resides outside of the lumen of flexible member 2207. Additionally, the diameter of tip 2208 has been correspondingly reduced to mate with the distal end of flexible member 2207. Yet another embodiment of combined steerable guide/dilation device 2200 is depicted in FIG. 22C. This embodiment of combined steerable guide/dilation device 2200 is similar to that shown in FIG. 22A, however, pullwire 2203 exits one of the lumens in multi-lumen tube 2202 through hole 2209 such that pullwire 2203 resides outside of the lumen of flexible member 2204. Alternatively (not shown), flexible members 2204 and 2207 may contain an inner and/or outer liner or may comprise a soft or flexible material fused to the member.

FIG. 23 depicts an alternative embodiment of the steerable balloon catheter 1400 shown in FIGS. 14A-14D. The steerable balloon catheter system 2300 comprises identical parts to combined steerable balloon catheter 1400 with the following exceptions: telescoping sheath 2100 is coaxially arranged over the balloon shaft 1412, aspiration port 1404 and aspiration tube 1410 have been removed, detents 2302 have been incorporated into balloon shaft 1412, aspiration seal 1408 has been removed, and guidewire valve 1406 has been replaced by guidewire valve 2301, and aspiration hub 1409 has been replaced by aspiration hub 2303. The listed alterations reflect the inclusion of a telescoping sheath 2100 that further comprises an aspiration port 2104. The presence of aspiration port 2104 on telescoping sheath 2100 could also optionally eliminate the need for the other aspiration port and associated components in the shell 1401. For example, guidewire valve 2301 does not comprise a channel or groove for retaining aspiration seal 1408, and aspiration hub 2303 does not comprise a feature for connecting to an aspiration tube.

Seal 2103 provides an air and/or fluid tight fit between telescoping sheath 2100 and balloon shaft 1412 and enable aspiration via aspiration port 2104. The detents 2302 comprise a path that traverses the circumference of the surface of the balloon shaft 1412. This allows the telescoping sheath 2100 to freely rotate 360 degrees clockwise or counter-clockwise about balloon shaft 1412. For example, the free rotation of telescoping sheath 2100 about balloon shaft 1412 enables the aspiration port 2104 to remain in a downward-facing direction (as shown in FIG. 23) irrespective of the rotational orientation of the balloon shaft 1412. The action of detents 2302 engaging the seal 2103 held in telescoping sheath 2100 may provide a tactile indication of the location of the telescoping sheath 2100 with respect to the balloon shaft 1412. In the distal position, telescoping sheath 2100 is positioned such that the balloon 1420 is covered by the telescoping sheath 2100. A retraction of the telescoping sheath 2100 in the proximal direction will uncover or unsheath balloon 1420 and may be accompanied by the tactile feedback of seal 2103 engaging detents 2302.

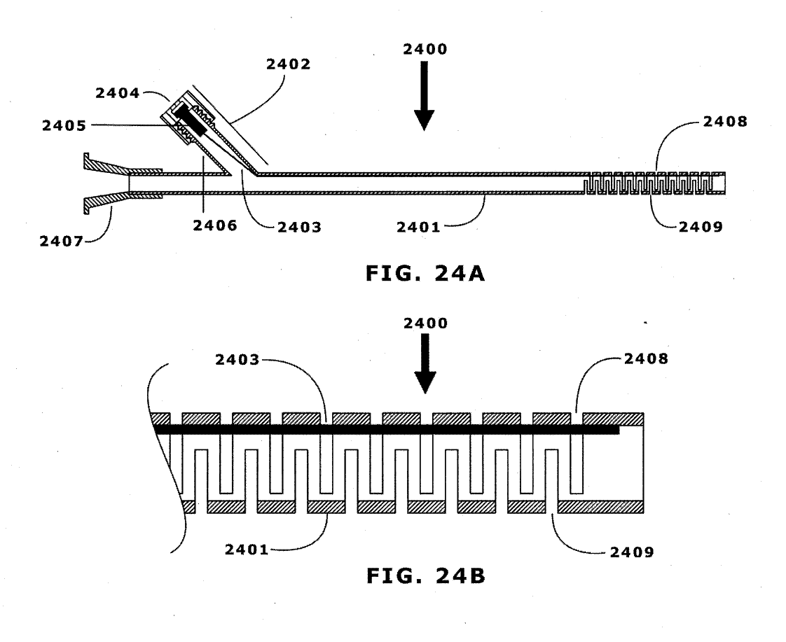

FIGS. 24A and 24B depict cross-sectional views of an embodiment of the invention comprising a steerable sheath 2400 with delineated component parts and features. Steerable sheath 2400 is further comprised of a sheath shaft 2401, control arm 2402, pullwire 2403, and proximal hub 2407. Sheath shaft 2401 further comprises a pattern of cuts 2408 and 2409 on its distal segment. Control arm 2402 further comprises control shaft 2406, pullwire hub 2405, and control knob 2404. Sheath shaft 2401 is an elongate member with proximal and distal ends and at least one lumen running therethrough that may be fabricated from materials known in the art including, but not limited to nylon, polyurethane, polycarbonate, polyimide, PET, PEEK, polyolefin, PTFE, Pebax, Delrin, polyethylene, stainless steel, nitinol, and combinations thereof. The sheath shaft 2401 may be sized to fit coaxially within a working device such as a balloon catheter. The distal portion of sheath shaft 2401 comprises two sets of cuts 2408 and 2409. It should also be understood by one of skill in the art that the use of two sets of internally identical cuts is exemplary only; additional arrangements, geometries, and permutations of cuts is well within the state of the art. As shown in FIG. 24B, the length of cuts 2408 is greater than the length of cuts 2409, and the spacing between cuts 2408 and 2409 is evenly distributed over the total number of cuts. It should be obvious to one of skill in the art that the relative and absolute lengths of both cuts 2408 and 2409 may be variable, furthermore, all of the cuts within the set of cuts 2408 and the set of cuts 2409 may not be identical. For example, the absolute length of cuts 2408 may decrease as the distal end of sheath shaft 2401 is approached. Additionally, the spacing between individual cuts in each set 2408 and 2409 as well as spacing between the span of sets 2408 and 2409 may be variable. Furthermore, while cuts 2408 and 2409 are shown as rectilinear in cross section, the shape of each cut in sets 2408 and 2409 may vary as well, including geometries such as but not limited to chevrons, triangles, curves, spirals, and the like. Cuts 2408 and 2409 may be fabricated using methods known in the art including, but not limited to laser cutting, grinding, electrical discharge machining, and the like. Alternatively (not shown), sheath shaft 2401 and/or cuts 2408 and 2409 may contain an inner and/or outer liner or may comprise a soft or flexible material fused to the member. Control arm 2402 is joined to sheath shaft 2401 via control shaft 2406. Control shaft 2406 is an elongate member with proximal and distal ends and at least one lumen running therethrough and may be fabricated from materials known in the art including, but not limited to nylon, polyurethane, polycarbonate, polyimide, PET, PEEK, polyolefin, PTFE, Pebax, Delrin, polyethylene, stainless steel, nitinol, and combinations thereof. Control arm 2402 is joined to sheath shaft 2401 using methods known in the art including, but not limited to welding, ultrasonic welding, adhesive bonding, crimping, overmolding, threading, and the like. The proximal segment of control arm 2402 is threaded in the example shown in FIG. 24A. Control knob 2404 is tapped such that the threads on control arm 2402 mate with the tapped portion of control knob 2404. Control knob 2404 may be fabricated from materials known in the art including, but not limited to nylon, polyurethane, polycarbonate, polyimide, PET, PEEK, polyolefin, PTFE, Pebax, Delrin, polyethylene, stainless steel, nitinol, and combinations thereof. Control knob 2404 further comprises a recess that houses pullwire hub 2405. Pullwire hub 2405 is sized such that it can rotate freely within the recess in control knob 2404. Pullwire hub 2405 may be fabricated from materials known in the art including, but not limited to nylon, polyurethane, polycarbonate, polyimide, PET, PEEK, polyolefin, PTFE, Pebax, Delrin, polyethylene, stainless steel, nitinol, and combinations thereof. The distal end of pullwire hub 2405 is joined to pullwire 2403 using methods known in the art including, but not limited to welding, ultrasonic welding, adhesive bonding, crimping, overmolding, use of a set screw, and the like. Pullwire 2403 runs through a lumen of control arm 2402 and a lumen of sheath shaft 2401. The distal end of pullwire 2403 is joined to the distal end of sheath shaft 2401 using methods known in the art including, but not limited to welding, ultrasonic welding, adhesive bonding, crimping, and the like. Alternatively (not shown), one or more additional pullwires may run between additional pullwire hubs and different points about the circumference of the distal end of sheath shaft 2401 to allow for control of over the three dimensional shape of the distal end of the steerable sheath 2400. The proximal end of sheath shaft 2401 is connected to proximal hub 2407 using methods known in the art including, but not limited to welding, ultrasonic welding, adhesive bonding, overmolding, threading/screwing, and the like. Proximal hub 2407 comprises at least one lumen and may be fabricated from materials known in the art including, but not limited to nylon, polyurethane, polycarbonate, polyimide, PET, PEEK, polyolefin, PTFE, Pebax, Delrin, polyethylene, stainless steel, nitinol, and combinations thereof. FIG. 24A illustrates proximal hub 2407 as a female luer lock, however, it should be clear to one of skill in the art that other components including, but not limited to female slip luers, Touhy-Borst valves, male luer locks, male slip luer, may be used interchangeably. While control arm 2402 is illustrated as comprising a tap and thread mechanism of controlling the relative position of the pullwire 2403 relative to sheath shaft 2401, it should be understood by those of skill in the art that similar mechanisms including, but not limited to linear slides, rack and pinions, gears, levers, winches, key/keyholes arrangements, direct threading of the pullwire, and the like may be used for this purpose. An aspiration port (not shown) may be optionally included on control arm 2402 and/or sheath shaft 2401 to allow for aspiration, flushing, or removal of fluid and/or tissue.

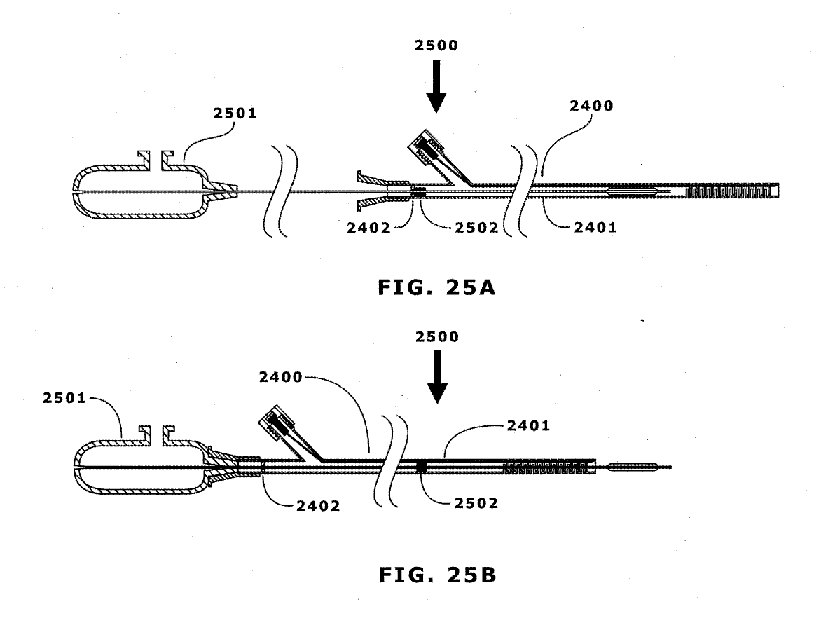

Another embodiment of the integrated balloon catheter and steerable telescoping sheath system 2500 comprising steerable sheath 2400 and an over-the-wire balloon catheter 2501 is shown in FIGS. 25A and 25B. In this example, the at least one lumen of sheath shaft 2401 is sized to accept balloon catheter 2501. Sheath shaft 2401 further comprises collar 2402. Locking collar 2402 may be joined to sheath shaft 2401 using methods known in the art including, but not limited to adhesive bonding, welding, ultrasonic welding, and the like. Locking collar 2402 may be fabricated from materials known in the art including, but not limited to nylon, polyurethane, polycarbonate, polyimide, PET, PEEK, polyolefin, PTFE, Pebax, Delrin, polyethylene, stainless steel, nitinol, and combinations thereof. Balloon catheter 2501 further comprises interference collar 2502 which is designed to integrate balloon catheter 2501 and steerable sheath 2400 into a single unit. Interference collar 2502 may be joined to balloon catheter 2501 using methods known in the art including, but not limited to adhesive bonding, welding, ultrasonic welding, and the like. Interference collar 2502 may be fabricated from materials known in the art including, but not limited to nylon, polyurethane, polycarbonate, polyimide, PET, PEEK, polyolefin, PTFE, Pebax, Delrin, polyethylene, stainless steel, nitinol, and combinations thereof. Locking collar 2402 and interference collar 2502 are arranged such that steerable sheath 2400 can not be separated from balloon catheter 2501. FIG. 25A depicts a cross-sectional view of the composition of the integrated balloon catheter and steerable telescoping sheath system 2500 in an initial configuration with the distal tip of steerable sheath 2400 extended past the distal tip of balloon catheter 2501. The integrated balloon catheter and a steerable telescoping sheath system 2500 may be inserted into the patient as configured in FIG. 25A and advanced such that the distal tip of the steerable sheath 2400 is at or near the target body lumen and/or ostium. The features of steerable sheath 2400 may then be used to adjust or deflect the angle of the distal tip of sheath shaft 2401 to a desired point and a guidewire may be advanced through the lumen of the over-the-wire balloon catheter 2501 and into and/or through the target body lumen and/or ostium. The steerable sheath 2400 may then be retracted proximally such that the integrated balloon catheter and steerable telescoping sheath system 2500 is configured as shown in FIG. 25B, wherein the balloon segment of balloon catheter 2501 is substantially uncovered or unsheathed. At this point the integrated balloon catheter and steerable telescoping sheath system 2500 may be advanced as a unit until the balloon traverses the target body lumen and/or ostium. The balloon may be inflated and deflated to treat the target body lumen and/or ostium, and the integrated balloon catheter and steerable telescoping sheath system 2500 may be retracted to remove the balloon from the target body lumen and/or ostium. The steerable sheath 2400 may be advanced such that the integrated balloon catheter and steerable telescoping sheath system 2500 returns to the configuration shown in FIG. 25A substantially covering or resheathing the deflated balloon. The guidewire may be retracted into the lumen of the over-the-wire balloon catheter 2501 and the integrated balloon catheter and steerable telescoping sheath system 2500 may be positioned to treat additional body lumens and/or ostia.

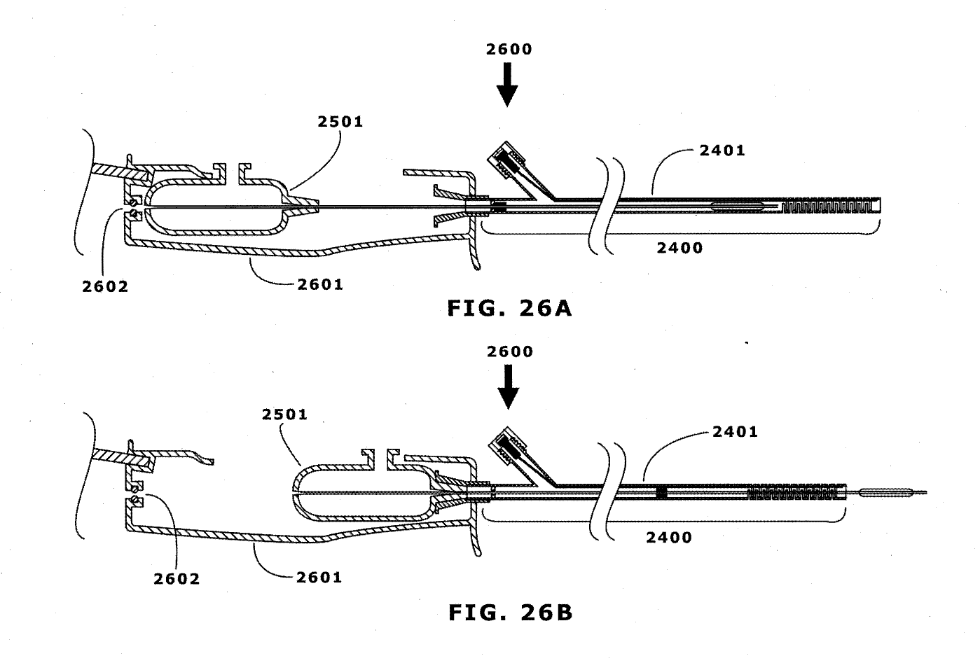

In another embodiment shown in FIGS. 26A and 26B, an integrated balloon catheter and steerable telescoping sheath system 2600 may comprise integrated balloon catheter and steerable telescoping sheath system 2500 and a shell 2601 that covers the hub of over-the-wire balloon catheter 2501 and the proximal hub 2407 of steerable sheath 2400. FIGS. 26A and 26B provide cross-sectional views of integrated balloon catheter and steerable telescoping sheath system 2600. The addition of shell 2601 would allow the balloon catheter 2501 to be advanced within sheath shaft 2401 and over a stationary guidewire into the target body lumen and/or ostium after placement of the guidewire. FIG. 26A shows the integrated balloon catheter and steerable telescoping sheath system 2600 with the balloon 2501 retracted fully proximal within the shell 2601. Shell 2601 further comprises a guidewire retaining member 2602. While guidewire retaining member 2602 is shown as an o-ring in FIGS. 26A and 26B, it should be understood by those of skill in the art that other components including, but not limited to Touhy-Borst valves, living hinges, iris valves, clamps, chucks, or combinations thereof. Guidewire retaining member 2602 enables insertion of an appropriately sized guidewire into the integrated balloon catheter and steerable telescoping sheath system 2600 and maintains the position of the guidewire with respect to shell 2601 when the guidewire is not actively advanced or retracted through the lumen of guidewire retaining member 2602. FIG. 26B shows the integrated balloon catheter and steerable telescoping sheath system 2600 with the hub of over-the-wire balloon catheter 2501 advanced fully proximal within the shell 2601. The distal end of over-the-wire balloon catheter 2501 extends past the distal end of wire guide shaft 2401. The integrated balloon catheter and steerable telescoping sheath system 2600 may be inserted into the patient as configured in FIG. 26A and advanced such that the distal tip of the integrated balloon catheter and steerable telescoping sheath system 2600 is at or near the target body lumen and/or ostium. The features of steerable sheath 2400 may be used to adjust the angle of the distal tip of sheath shaft 2401 to a desired point and a guidewire may be advanced through the guidewire retaining member 2602, into the lumen of the over-the-wire balloon catheter 2501, and into and/or through the target body lumen and/or ostium. The hub of over-the-wire balloon catheter 2501 may then be advanced within shell 2601 such that the integrated balloon catheter and steerable telescoping sheath system 2600 is configured as shown in FIG. 26B and the balloon component of the over-the-wire balloon catheter 2501 is placed within the target body lumen and/or ostium. The balloon may be inflated and deflated to treat the target body lumen and/or ostium, and the hub of over-the-wire balloon catheter 2501 may be retracted fully distally within shell 2601 such the configuration of the integrated balloon catheter and steerable telescoping sheath system 2600 returns to that shown in FIG. 26A, removing balloon of over-the-wire balloon catheter 2501 from the target body lumen and/or ostium. The guidewire may be retracted into the lumen of the over-the-wire balloon catheter 2501 and the integrated balloon catheter and steerable telescoping sheath system 2600 may be positioned to treat additional body lumens and/or ostia.

In all the embodiments listed in this invention, resolution of the tip indication mechanisms could be described in the device instructions for use and could vary depending on the resolution required for a particular procedure. As an example, in sinus ostium dilatation procedures the inscription and/or the detents or clicks could adjusted such that each indicator positioned the tip at various angles starting at approximately zero (0) degrees to approximately ninety degrees in approximately thirty degree increments. The first indicator would then be zero, the second could be at thirty (30) degrees, the third could be at sixty (60) degrees and the final indicator or detent could be at ninety (90) degrees. It is obvious that there an infinite number of permutations of where these indicators and detents could be set and the previous description provides only example without placing limitations on constructing other permutations. Additionally, it is understood that positioning the control hub between indicator marks (e.g. between the 30 and 60 degree indicators) would produce an approximate tip angle ranging between 30 and 60 degrees.

### Methods of Use

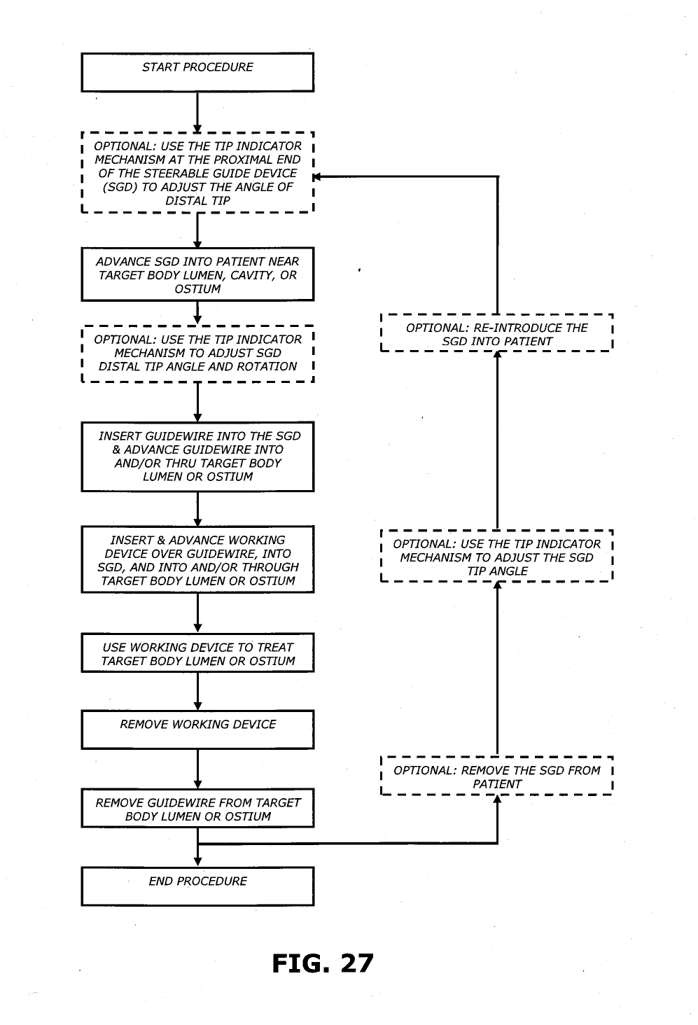

FIG. 27 depicts a flowchart describing an embodiment of a method for using the steerable guide devices of the invention as described in FIGS. 1-13 and 24 to treat one or more body lumens and/or ostia. For example, the method described in FIG. 27 can be followed to treat multiple paranasal sinuses; the method comprising optionally employing the tip indicator mechanism to adjust the angle of the distal tip of the steerable guide device prior to inserting the device into a subject. Using endoscopic, fluoroscopic, computed tomographic, infrared, magnetic, ultrasonic, and/or electromagnetic guidance if desired, the steerable guide system is positioned in proximity to the sinus ostium that is the target of the medical treatment. If needed, the tip indicator mechanism is used to further adjust the angle or rotational orientation of the distal tip of the steerable guide device. An appropriately sized guidewire is inserted into a lumen of the steerable guide device and passed into and/or through the lumen of the target sinus ostium. A working device such as an over-the-wire balloon catheter may be loaded over the guidewire and advanced through the lumen of the steerable guide device until the balloon is resident within the target sinus ostium. The balloon is inflated to dilate the target sinus ostium, after which the balloon is deflated and the balloon catheter is removed from the subject. The guidewire is subsequently removed from the treated sinus ostium. At this point, the steerable guide device may be removed from the subject, the tip indicator mechanism may be used to adjust the angle and or rotation of the tip of the steerable guide device, and the steerable guide device may be reinserted into the patient. This may occur when treating right and left paranasal sinuses, for example. Alternatively, the steerable guide device may remain resident in the paranasal sinus after treatment of the initial sinus ostium and guided to a position at or near a second ipsilateral target sinus ostium and the process may be repeated. While the treatment of multiple sinus ostia serves to illustrate the method of FIG. 27, it should be obvious to one of skill in the art that these devices and corresponding methods are applicable to various surgical procedures, such as balloon atherectomy and the like.

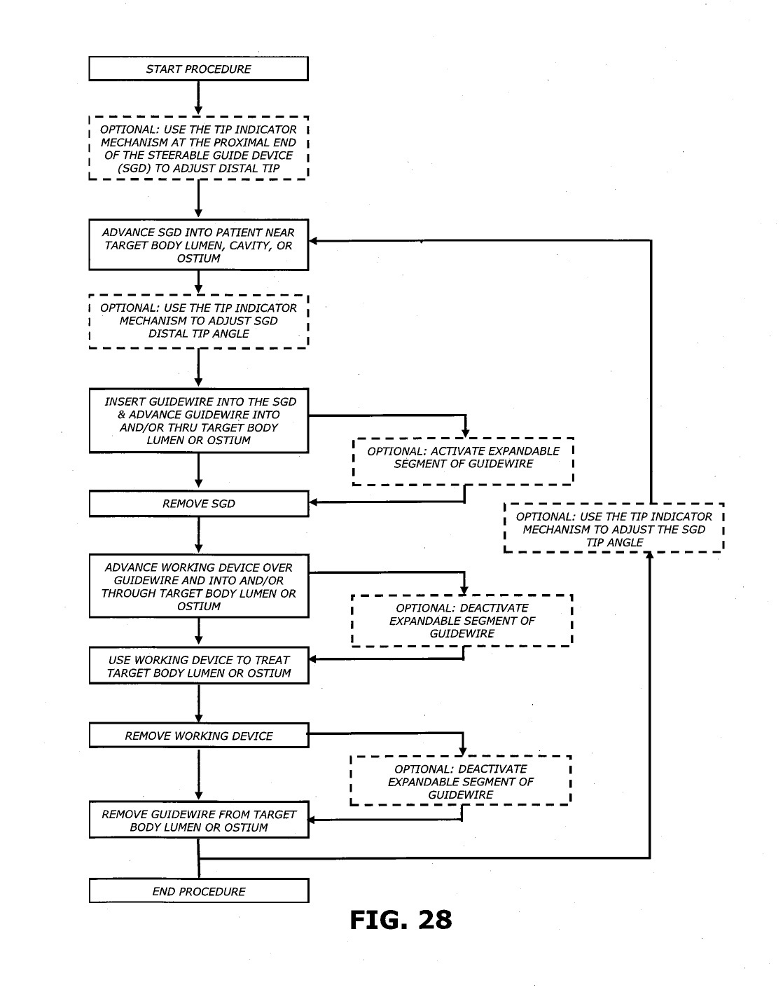

FIG. 28 depicts a flowchart describing an alternative embodiment of a method for using the steerable guide devices of the invention as described in FIGS. 1-13 and 24 to treat one or more body lumens or ostia. It may be desired to remove the steerable guide device from the subject prior to introducing a working device such as a balloon catheter, stent, or similar tool over a guidewire that has been placed in a target body lumen and/or ostium. It may be advantageous for the guidewire to have an expandable segment to aid in maintaining placement of the guidewire in the target body lumen and/or ostium during or after removal of the steerable guide device. As an example, the method described in FIG. 28 can be followed to treat multiple paranasal sinuses; the method comprising optionally employing the tip indicator mechanism to adjust the angle of the distal tip of the steerable guide device prior to inserting the device into a subject. Using endoscopic, fluoroscopic, computed tomographic, infrared, magnetic, ultrasonic, and/or electromagnetic guidance if desired, the steerable guide system is positioned in proximity to the sinus ostium that is the target of the medical treatment. If needed, the tip indicator mechanism is used to further adjust the angle or rotational orientation of the distal tip of the steerable guide device. An appropriately sized guidewire is inserted into a lumen of the steerable guide device and passed into and/or through the lumen of the target sinus ostium. Optionally, if the guidewire comprises an expandable segment, and the expandable segment has traversed the sinus ostia, the operator may activate the expandable segment of the guidewire such that the expanded segment maintains the position of the guidewire in the ostium. The steerable guide device is then removed from subject. A working device such as an over-the-wire balloon catheter may be loaded over the guidewire until the balloon is resident within the target sinus ostium. Optionally, if the guidewire comprises an expandable segment, the operator may deactivate the expandable segment of the guidewire. The balloon is inflated to dilate the target sinus ostium, after which the balloon is deflated and the balloon catheter is removed from the subject. Optionally, if the guidewire comprises an expandable segment, and the expandable segment remains active, the operator may deactivate the expandable segment of the guidewire. The guidewire is subsequently removed from the treated sinus ostium. At this point, the tip indicator mechanism may be used to adjust the angle and or rotation of the tip of the steerable guide device, and the steerable guide device may be reinserted into the patient. This may occur when treating right and left paranasal sinuses, for example. Alternatively, the steerable guide device may remain resident in the paranasal sinus after treatment of the initial sinus ostium and guided to a position at or near a second ipsilateral target sinus ostium and the process may be repeated. While the treatment of multiple sinus ostia serves to illustrate the method of FIG. 28, it should be obvious to one of skill in the art that these devices and corresponding methods are applicable to various surgical procedures, such as balloon atherectomy and the like.

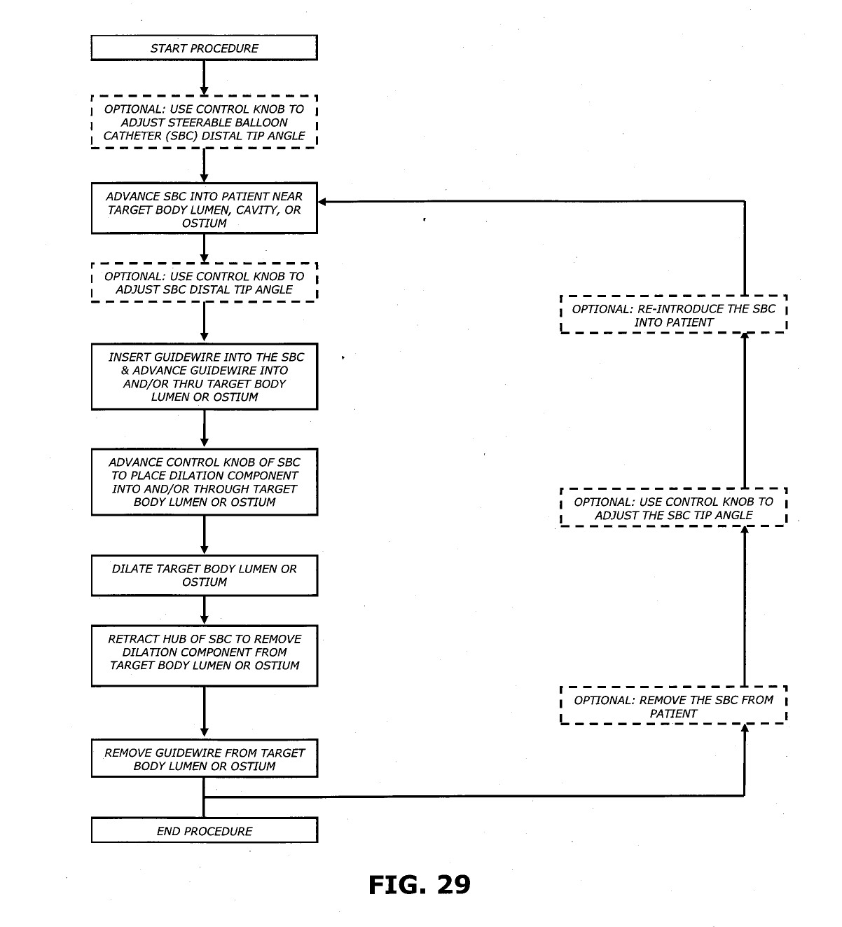

FIG. 29 depicts a flowchart describing an embodiment of a method for using the steerable balloon catheter of the invention as described in FIGS. 14A-14D to treat one or more body lumens and/or ostia. As an example, the method described in FIG. 29 can be followed to treat multiple paranasal sinuses; the method comprising optionally adjusting the deflection of the distal tip of the steerable guide catheter prior to insertion of the steerable guide catheter into a subject. Under endoscopic, fluoroscopic, computed tomographic, infrared, magnetic, ultrasonic, and/or electromagnetic guidance if desired, the steerable balloon catheter is positioned in proximity to the sinus ostium that is the target of the medical treatment. An appropriately sized guidewire is inserted into a lumen of the steerable balloon catheter and passed into and/or through the lumen of the target sinus ostium. The control knob of the steerable balloon catheter is then advanced distally within the shell of the steerable balloon catheter to position the balloon within the target sinus ostium. The balloon is inflated to dilate the target sinus ostium, after which the balloon is deflated and the control knob of the steerable balloon catheter is retracted proximally to withdraw the balloon from the treated sinus ostium. The guidewire is subsequently removed from the treated sinus ostium. At this point, the steerable balloon catheter may be removed from the subject, the control knob may be used to adjust the angle and/or rotation of the tip of the steerable balloon catheter to a desired position, and the steerable balloon catheter may be reinserted into the patient. This may occur when treating right and left paranasal sinuses, for example. Alternatively, the steerable balloon catheter may remain resident in the paranasal sinus after treatment of the initial sinus ostium and guided to a position at or near a second ipsilateral target sinus ostium and the process may be repeated. While the treatment of multiple sinus ostia serves to illustrate the method of FIG. 29, it should be obvious to one of skill in the art that these devices and corresponding methods are applicable to various surgical procedures, such as balloon atherectomy and the like.

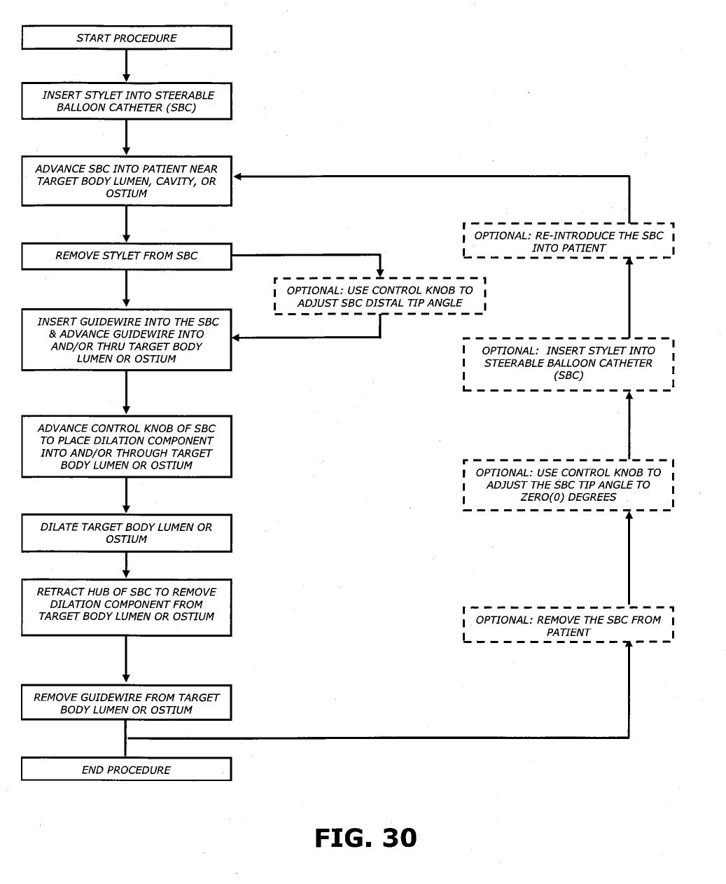

FIG. 30 depicts a flowchart describing an embodiment of a method for using the steerable balloon catheter of the invention as described in FIGS. 14A-14E to treat one or more body lumens and/or ostia. As an example, the method described in FIG. 30 can be followed to treat multiple paranasal sinuses; the method comprising inserting a relatively stiff stylet into a lumen of the steerable balloon catheter prior to prior to inserting the device into a subject. Alternatively, the steerable balloon catheter may be supplied to the operator with the stylet already placed in a lumen of the steerable balloon catheter. Under endoscopic, fluoroscopic, computed tomographic, infrared, magnetic, ultrasonic, and/or electromagnetic guidance if desired, the steerable balloon catheter is positioned in proximity to the sinus ostium that is the target of the medical treatment. The steerable balloon catheter may be used to perform retraction of tissues such as the middle turbinate as required. The stylet is removed to enable the control knob to adjust the angle or rotational orientation of the distal tip of the steerable balloon catheter. An appropriately sized guidewire is inserted into a lumen of the steerable balloon catheter and passed into and/or through the lumen of the target sinus ostium. The control knob of the steerable balloon catheter is then advanced distally within the shell of the steerable balloon catheter to position the balloon within the target sinus ostium. The balloon is inflated to dilate the target sinus ostium, after which the balloon is deflated and the control knob of the steerable balloon catheter is retracted proximally to withdraw the balloon from the treated sinus ostium. The guidewire is subsequently removed from the treated sinus ostium. At this point, the steerable balloon catheter may be removed from the subject, the control knob may be used to adjust the angle and/or rotation of the tip of the steerable balloon catheter to a zero (0) degree angle, the stylet may be re-inserted into the guidewire lumen of the steerable balloon catheter, and the steerable balloon catheter may be reinserted into the patient. This may occur when treating right and left paranasal sinuses, for example. Alternatively, the steerable balloon catheter may remain resident in the paranasal sinus after treatment of the initial sinus ostium and guided to a position at or near a second ipsilateral target sinus ostium and the process may be repeated. While the treatment of multiple sinus ostia serves to illustrate the method of FIG. 30, it should be obvious to one of skill in the art that these devices and corresponding methods are applicable to various surgical procedures, such as balloon atherectomy and the like.

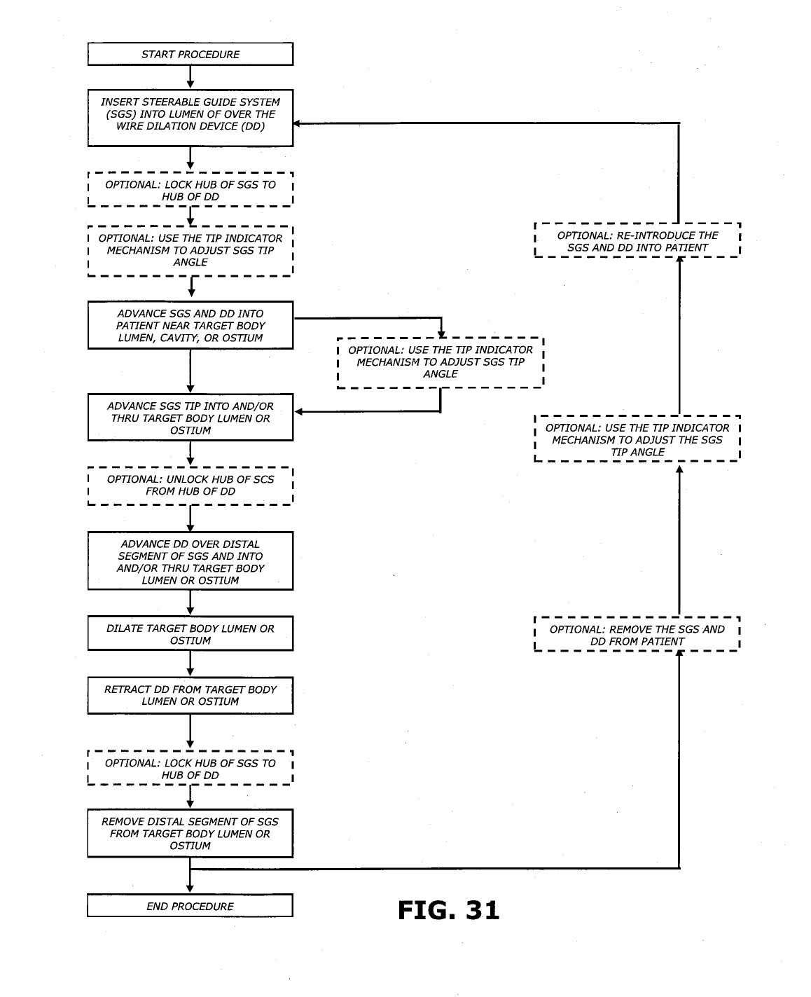

FIG. 31 depicts a flowchart describing an alternative embodiment of a method for using the steerable guide systems of the invention as described in FIGS. 15 and 16 to treat one or more body lumens and/or ostia. As an example, the method described in FIG. 31 can be followed to treat multiple paranasal sinuses; the method comprising inserting the steerable guide device into the lumen of an over-the-wire balloon catheter. The operator may optionally reversibly lock the hub of the steerable guide system to the hub of the balloon catheter. The tip indicator mechanism may be used to adjust the angle of the distal tip of the steerable guide system prior to inserting the steerable guide system and balloon catheter as a single unit into a subject. Using endoscopic, fluoroscopic, computed tomographic, infrared, magnetic, ultrasonic, and/or electromagnetic guidance if desired, the steerable guide system and balloon catheter are positioned such that the tip of the steerable guide system is in proximity to the sinus ostium that is the target of the medical treatment. If needed, the tip indicator mechanism is used to further adjust the angle or rotational orientation of the distal tip of the steerable guide system. The tip of the steerable guide system is advanced into and/or through the target sinus ostium. If the steerable guide system hub and balloon catheter hub have been reversibly locked to each other, the operator may free the balloon catheter hub from steerable guide system hub. The balloon catheter is then advanced distally over the steerable guide system until the balloon is within the target sinus ostium. The balloon is inflated to dilate the target sinus ostium, after which the balloon is deflated and the balloon catheter is retracted distally to withdraw the balloon from the treated sinus ostium. The user may then optionally reversibly lock the hub of the steerable guide system to the hub of the balloon catheter from the subject. The distal segment of the steerable guide system is then withdrawn from the treated sinus ostium. At this point, the steerable guide system and balloon catheter may be removed from the subject as a unit, the tip indicator mechanism may be used to adjust the angle and or rotation of the tip of the steerable guide system, and the steerable guide system and balloon catheter may be reinserted into the patient as a unit. This may occur when treating right and left paranasal sinuses, for example. Alternatively, the steerable guide system and balloon catheter may remain resident in the paranasal sinus after treatment of the initial sinus ostium and guided to a position at or near a second ipsilateral target sinus ostium and the process may be repeated. While the treatment of multiple sinus ostia serves to illustrate the method of FIG. 31, it should be obvious to one of skill in the art that these devices and corresponding methods are applicable to various surgical procedures, such as balloon atherectomy and the like.

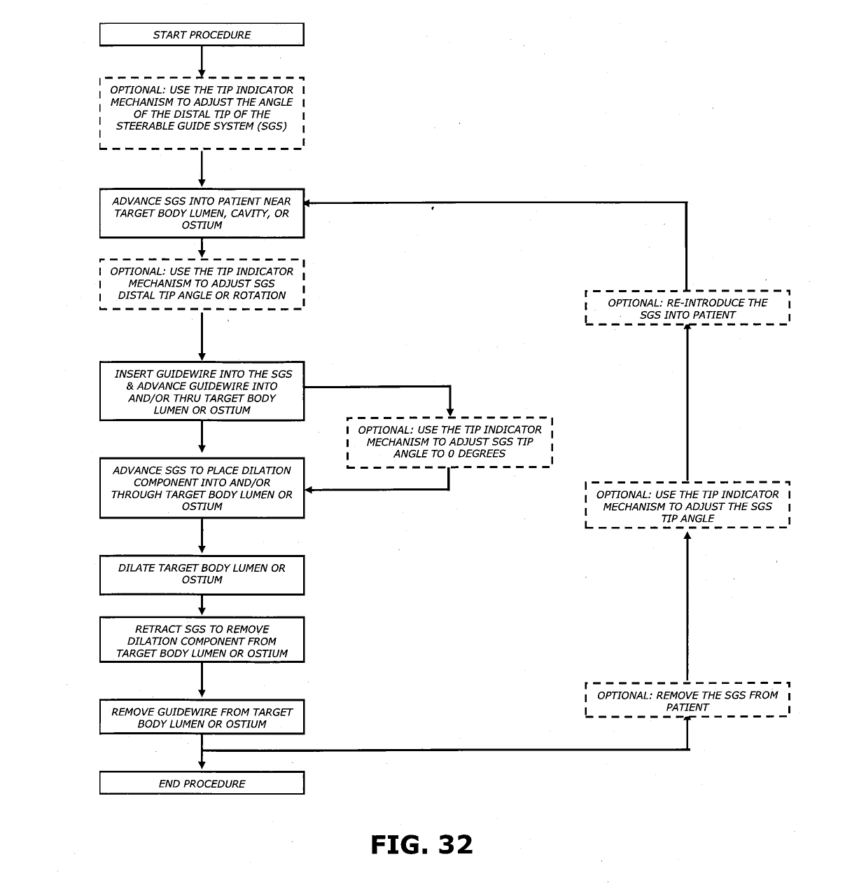

FIG. 32 depicts a flowchart describing an alternative embodiment of a method for using the steerable guide systems of the invention as described in FIGS. 19 and 20 to treat one or more body lumens and/or ostia. As an example, the method described in FIG. 32 can be followed to treat multiple paranasal sinuses; the method comprising optionally adjusting the angle of the distal tip of the steerable guide system to a desired position. Using endoscopic, fluoroscopic, computed tomographic, infrared, magnetic, ultrasonic, and/or electromagnetic guidance if desired, the steerable guide system is advanced into the subject and positioned such that the tip is in proximity to the sinus ostium that is the target of the medical treatment. The steerable guide system may be used to perform retraction of tissues such as the middle turbinate as required. If needed, the tip indicator mechanism is used to further adjust the angle or rotational orientation of the distal tip of the steerable guide system. An appropriately sized guidewire is inserted into a lumen of the steerable guide system and passed into and/or through the lumen of the target sinus ostium. The steerable guide system is then advanced proximally over the guidewire to position the balloon within the target sinus ostium. The balloon is inflated to dilate the target sinus ostium, after which the balloon is deflated and the steerable guide system is retracted distally to withdraw the balloon from the treated sinus ostium. The guidewire is subsequently removed from the treated sinus ostium. At this point, the steerable guide system may be removed from the subject, the tip indicator mechanism may be used to adjust the angle and or rotation of the tip of the steerable guide system, and the steerable guide system may be reinserted into the patient. This may occur when treating right and left paranasal sinuses, for example. Alternatively, the steerable guide catheter and balloon catheter may remain resident in the paranasal sinus after treatment of the initial sinus ostium and guided to a position at or near a second ipsilateral target sinus ostium and the process may be repeated. While the treatment of multiple sinus ostia serves to illustrate the method of FIG. 32, it should be obvious to one of skill in the art that these devices and corresponding methods are applicable to various surgical procedures, such as balloon atherectomy and the like.

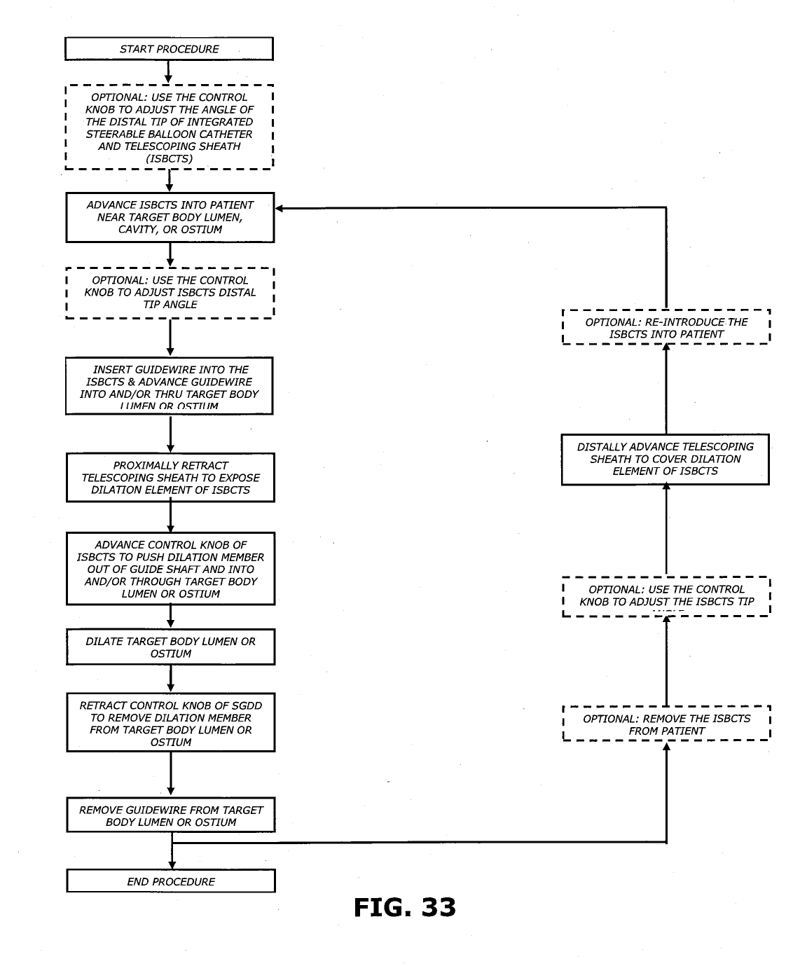

FIG. 33 depicts a flowchart describing an embodiment of a method for using the integrated steerable balloon catheter and telescoping sheath of the invention as described in FIG. 23 to treat one or more body lumens and/or ostia. As an example, the method described in FIG. 33 can be followed to treat multiple paranasal sinuses. Under endoscopic or fluoroscopic guidance if desired, the integrated steerable balloon catheter and telescoping sheath is positioned in proximity to the sinus ostium that is the target of the medical treatment. The integrated steerable balloon catheter and telescoping sheath may be used to perform retraction of tissues such as the middle turbinate as required. If needed, the control knob is used to further adjust the angle and/or rotational orientation of the distal tip of the integrated steerable balloon catheter and telescoping sheath. An appropriately sized guidewire is inserted into a lumen of the integrated steerable balloon catheter and telescoping sheath and passed into and/or through the lumen of the target sinus ostium. The telescoping sheath is retracted proximally along the balloon shaft to expose or unsheath the balloon. The control knob of the integrated steerable balloon catheter and telescoping sheath is then advanced distally within the shell of the integrated steerable balloon catheter and telescoping sheath to position the balloon within the target sinus ostium. The balloon is inflated to dilate the target sinus ostium, after which the balloon is deflated and the control knob of the integrated steerable balloon catheter and telescoping sheath is retracted proximally to withdraw the balloon the treated sinus ostium. The guidewire is subsequently removed from the treated sinus ostium. At this point, the integrated steerable balloon catheter and telescoping sheath may be removed from the subject, and the control knob may be used to adjust the angle and or rotation of the tip of the integrated steerable balloon catheter and telescoping sheath. The telescoping sheath is advanced distally to recover or resheath the balloon and the integrated steerable balloon catheter and telescoping sheath may be reinserted into the patient. This may occur when treating right and left paranasal sinuses, for example. Alternatively, the integrated steerable balloon catheter and telescoping sheath may remain resident in the paranasal sinus after treatment of the initial sinus ostium and guided to a position at or near a second ipsilateral target sinus ostium and the process may be repeated. While the treatment of multiple sinus ostia serves to illustrate the method of FIG. 33, it should be obvious to one of skill in the art that these devices and corresponding methods are applicable to various surgical procedures, such as balloon atherectomy and the like.

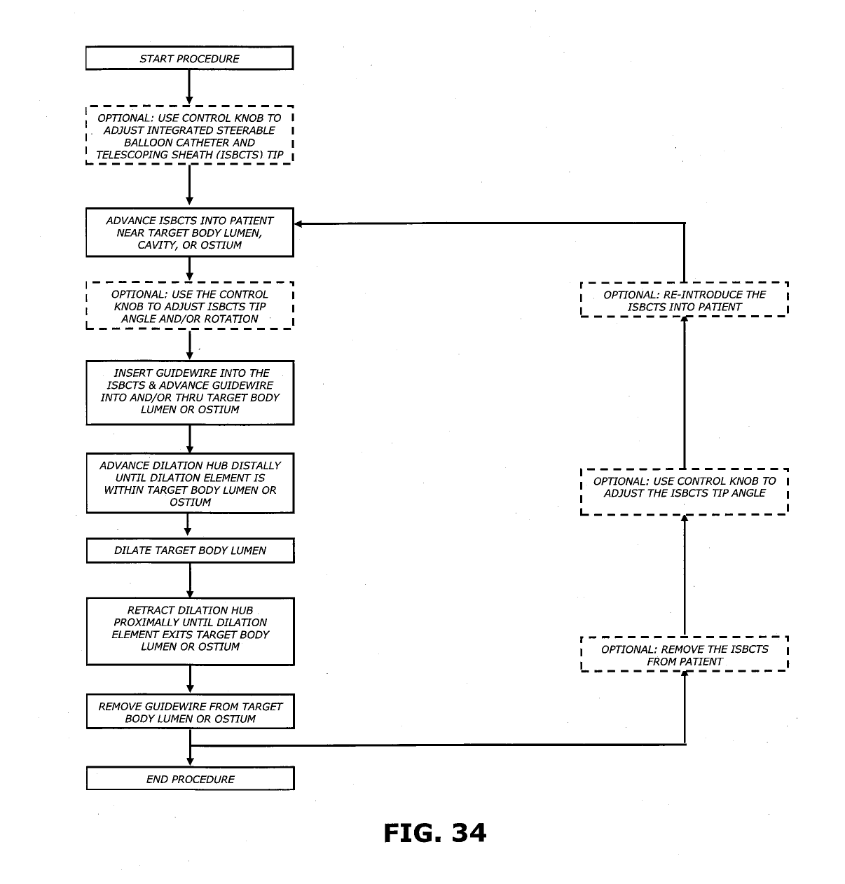

FIG. 34 depicts a flowchart describing an alternative embodiment of a method for using the integrated steerable balloon catheter and telescoping sheath of the invention as described in FIGS. 25 and 26 to treat one or more body lumens and/or ostia. As an example, the method described in FIG. 34 can be followed to treat multiple paranasal sinuses. The control knob may be used to adjust the angle of the distal tip of the integrated steerable balloon catheter and telescoping sheath prior to inserting the device into a subject. Using endoscopic, fluoroscopic, computed tomographic, infrared, magnetic, ultrasonic, and/or electromagnetic guidance if desired, the integrated steerable balloon catheter and telescoping sheath is positioned in proximity to the sinus ostium that is the target of the medical treatment. If needed, the control knob is used to further adjust the angle and/or rotational orientation of the distal tip of the integrated steerable balloon catheter and telescoping sheath. An appropriately sized guidewire is inserted into a lumen of the integrated steerable balloon catheter and telescoping sheath and passed into and/or through the lumen of the target sinus ostium. The balloon hub of the integrated steerable balloon catheter and telescoping sheath is then advanced distally to position the balloon within the target sinus ostium. The balloon is inflated to dilate the target sinus ostium, after which the balloon is deflated and the balloon hub of the integrated steerable balloon catheter and telescoping sheath is retracted proximally to withdraw the balloon from the treated sinus ostium. The guidewire is subsequently removed from the treated sinus ostium. At this point, the integrated steerable balloon catheter and telescoping sheath may be removed from the subject, and the control knob may be used to adjust the angle and or rotation of the tip of the integrated steerable balloon catheter and telescoping sheath, and the integrated steerable balloon catheter and telescoping sheath may be reinserted into the patient. This may occur when treating right and left paranasal sinuses, for example. Alternatively, the integrated steerable balloon catheter and telescoping sheath may remain resident in the paranasal sinus after treatment of the initial sinus ostium and guided to a position at or near a second ipsilateral target sinus ostium and the process may be repeated. While the treatment of multiple sinus ostia serves to illustrate the method of FIG. 34, it should be obvious to one of skill in the art that these devices and corresponding methods are applicable to various surgical procedures, such as balloon atherectomy and the like.

The preceding merely illustrates the principles of the invention. It will be appreciated that those skilled in the art will be able to devise various arrangements, which, although not explicitly described or shown herein, embody the principles of the invention, and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1-12. (canceled)

13. A steerable balloon catheter comprising;
a shell enclosing a balloon control hub, wherein the balloon control hub can move with respect to the shell;
a multi-lumen tubing having a proximal end, a distal end, and at least two lumens coaxially disposed within a balloon shaft having a proximal end, a distal end, and at least one lumen, wherein the distal end of the multi-lumen tubing extends beyond the distal end of the balloon shaft;
an expandable balloon element;
a flexible element having a proximal end, a distal end, and at least one lumen, wherein the proximal end is joined to the distal end of the multi-lumen tubing;
a distal tip having a proximal end, a distal end, and at least one lumen, wherein the proximal end is joined to the distal end of the flexible element;
a wire having a proximal end, a distal end, and a cross-sectional geometry residing in at least a portion of at least one of the lumens of the multi-lumen tubing, wherein the distal end of the wire is joined to the distal end of the flexible element and/or the proximal end of the distal tip; and
a control knob disposed on the balloon control hub enabling a tensile or compressive load to be applied to the wire.

14. The steerable balloon catheter according to claim 13, wherein the distal tip is substantially less rigid than the multi-lumen tubing.

15. The steerable balloon catheter according to claim 13, wherein the distal tip is atraumatic.

16. The steerable balloon catheter according to claim 13, wherein the distal tip cannot support a compressive load sufficient to cross into and/or through an opening into a diseased paranasal sinus.

17. The steerable balloon catheter according to claim 13, wherein the control knob is joined to the wire via a mechanism comprising a rack and pinion, an internal and/or external screw thread, a detent, a ratchet, a living hinge, a spring and ball, a key and keyway, and/or a winch.

18. The steerable balloon catheter according to claim 13, wherein the shell further comprises at least one marking denoting the potential angles of deflection of the distal tip of the steerable balloon catheter.

19. The steerable balloon catheter according to claim 18, wherein the control knob further comprises an indicator that aligns with at least one marking on the shell to denote the current angle of deflection of the distal tip of the steerable balloon catheter.

20. The steerable balloon catheter according to claim 13, wherein the control knob further comprises at least one marking denoting the potential angles of deflection of the distal tip of the steerable balloon catheter.

21. The steerable balloon catheter according to claim 20, wherein the shell further comprises an indicator that aligns with at least one marking on the control knob to denote the current angle of deflection of the distal tip of the steerable balloon catheter.

22. The steerable balloon catheter according to claim 13, wherein the expandable balloon element regrooms during deflation.

23. The steerable balloon catheter according to claim 22, wherein the balloon shaft rotates and/or translates about the multi-lumen tubing to regroom the expandable balloon element.

24. The steerable balloon catheter according to claim 22, wherein the proximal end of the expandable balloon element is rotated about the longitudinal axis of the expandable balloon element with respect to the distal end of the expandable balloon element in the collapsed state.

25. The steerable balloon catheter according to claim 22, wherein the expandable balloon element is under no load or is under tension in the collapsed state.

26. The steerable balloon catheter according to claim 13, wherein at least one lumen of the multi-lumen tubing, and/or at least one lumen of the flexible element, and/or at least one lumen of the distal tip is sized to accept a guidewire.

27. The steerable balloon catheter according to claim 13, wherein the shell further comprises a flexible handle extension and a handle.

28. The steerable balloon catheter according to claim 27, wherein the flexible handle extension maintains a stable position after modification of the shape of the flexible handle extension.

29. The steerable balloon catheter according to claim 27, wherein the handle is not axissymetric.

30. The steerable balloon catheter according to claim 27, wherein the handle further comprises convex and/or concave contours.

31. The steerable balloon catheter according to claim 27, wherein the handle further comprises at least one soft element to enable comfort and/or stability during a medical procedure.

32. The steerable balloon catheter according to claim 13, wherein the shell further comprises an aspiration port that is in communication with at least one lumen of the multi-lumen tubing.

33. The steerable balloon catheter according to claim 13, wherein the shell further comprises a guidewire retaining valve.

34. The steerable balloon catheter according to claim 13, wherein the shell further comprises a window allowing access to the control knob.

35. The steerable balloon catheter according to claim 13, wherein the shell further comprises at least one flange.

36. The steerable balloon catheter according to claim 13, wherein the flange or flanges are of sufficient strength and geometry to advance and/or retract the balloon hub with respect to the shell.

37. The steerable balloon catheter according to claim 13, wherein the shell further comprises an inflation port that is in communication with the expandable balloon element.

38. The steerable balloon catheter according to claim 13, further comprising a stylet having a proximal end and a distal end.

39. The steerable balloon catheter according to claim 13, wherein the stylet is coaxially disposed within at least one lumen of the multi-lumen tubing, and/or at least one lumen of the flexible element, and/or at least one lumen of the distal tip.

40. The steerable balloon catheter according to claim 38, wherein the stylet is removable.

41. The steerable balloon catheter according to claim 38, wherein the distal end of the stylet does not extend past the distal end of the distal tip of the steerable balloon catheter.

42. The steerable balloon catheter according to claim 38, wherein the proximal end of the stylet further comprises a feature the interferes with the guidewire retaining valve and/or shell and prevents over-insertion of the stylet into at least one lumen of the multi-lumen tubing, and/or at least one lumen of the flexible element, and/or at least one lumen of the distal tip of the steerable balloon catheter.

43. The steerable balloon catheter according to claim 38, wherein the stylet increases the rigidity or stiffness of the steerable balloon catheter.

44. The steerable balloon catheter according to claim 13, wherein the steerable balloon catheter further comprises at least one marker that may provide visualization under image guidance systems.

45. The steerable balloon catheter according to 44, wherein the steerable balloon catheter further comprises at least one marker that may provide visualization in concert with image guidance systems that utilize magnetic, electromagnetic, fluoroscopic, computed tomographic, magnetic resonance, infrared, or ultrasonic modalities.

46-79. (canceled)
